(19) United States
(12) Patent Application Publication
(10) Pub. No.: US 2025/0270316 A1
(43) Pub. Date: Aug. 28, 2025

(54) PHARMACEUTICAL COMBINATION AND USE THEREOF

(71) Applicant: TIANJIN LIPOGEN TECHNOLOGY CO., LTD, Tianjin (CN)

(72) Inventors: Chenguang WANG, Tianjin (CN); Xiaojiao ZHANG, Tianjin (CN); Xiaomeng DANG, Tianjin (CN); Jin ZHANG, Tianjin (CN); Yuting FU, Tianjin (CN); Yihan WANG, Tianjin (CN); Chao WANG, Tianjin (CN)

(21) Appl. No.: 18/574,085

(22) PCT Filed: Jun. 29, 2022
(86) PCT No.: PCT/CN2022/102169

§ 371 Date: Dec. 26, 2023

(30) Foreign Application Priority Data
Jul. 1, 2021 (CN) 202110751196.8

(51) Int. Cl. C07K 16/28; A61K 9/00; A61K 31/437; A61K 45/06; A61P 35/00

(52) U.S. Cl. CPC C07K 16/2803; A61K 9/0019; A61K 31/437; A61K 45/06; A61P 35/00; C07K 2317/51; C07K 2317/565

(57) ABSTRACT

A pharmaceutical combination and a use thereof. The pharmaceutical combination comprises a PD-1 inhibitor and/or a PD-L1 immune checkpoint inhibitor; and a Toll Like Receptor (TLR) agonist.

### CROSS-REFERENCES TO RELATED APPLICATION

The present application is a National Stage of International Patent Application No: PCT/CN2022/102169 filed on Jun. 29, 2022, which claims the benefit of the priority of the Chinese patent application with the application Ser. No. 20/211,0751196.8, filed to the China National Intellectual Property Administration on Jul. 1, 2021, the entire content of which is incorporated in this application by reference.

### SEQUENCE LISTING

The present application contains a Sequence Listing which has been submitted electronically in ASCII format and is hereby incorporated by reference in its entirety. Said ASCII copy is named_Sequence_Listing.txt and is 33,365 bytes in size, and contains SEQ ID NO: 1 to SEQ ID NO: 44 which are identical to the sequence listing filed in the corresponding international application No. PCT/CN2022/102169 filed on Jun. 29, 2022.

### TECHNICAL FIELD

The disclosure relates to the field of biomedicine, specifically to development and application of a pharmaceutical combination.

### BACKGROUND

Over the past decade, targeting PD-1 and PD-L1 has opened up a new era for clinical treatment of cancers. However, for many types of tumors, an immune checkpoint inhibitor monotherapy has poor efficacy. Even for immune responsive tumors, sustained clinical benefits have not been achieved in most patients. The vast majority of patients develop primary or acquired therapeutic drug resistance. In most cases, immunotherapy drug resistance can be attributed to the presence of immunosuppressive TME and insufficient activation of T cell anti-tumor effects due to a decrease in the number of immune cells in vivo. Therefore, compared to monotherapy, it is particularly important to actively seek new therapeutic strategies capable of reducing immune suppression in tumor environments or enhancing the response of cytotoxic cells to tumors.

The PD-L1/PD-1 signaling pathway is a very important co-inhibitory signaling pathway in the immune response. Researches have shown that when PD-L1 binds to PD-1, it will complement protein-tyrosine-phosphatases SHP-1 and SHP-2 with SH2 domains. These two phosphatases can reduce the extent of phosphorylation of the immunoreceptor tyrosine-based activation motif (ITAM) of CD3 ζ strand, weaken the activation of ZAP-70, and inhibit TCR downstream signaling, thereby playing a co-inhibitory role in T cell activation, and the autoimmune injury due to excessive activation of effector T cells can be prevented through such negative regulatory effect.

Researches have shown that some viral infections are also closely related to the PD-L1/PD-1 signaling pathway. For example, in chronic HIV infections, PD-1 was found to be highly expressed on the surface of CD8+T cells that specifically recognize HIV. The virus activates the PD-L1/PD-1 signaling pathway, through which the activity of CD8+T cells that specifically recognize HIV is inhibited, and the secretion ability of cytokines and the proliferation ability of T cells themselves are greatly weakened, leading to adaptive immune dysfunctions.

Toll Like Receptors (TLRs) are important Pattern Recognition Receptors (PRRs) in innate immunity, and specifically recognize a conservative Pathogen Associated Molecular Pattern (PAMP) on the surfaces of pathogenic microorganisms and some endogenous ligands, known as Damage-Associated Molecular Patterns (DAMPs). By means of MyD88 dependent or non-dependent pathways, signal transduction is caused, an organism is induced to produce type I interferon and various chemokines and inflammatory cytokines (such as IL-1, IL-6, TNF-α, etc.), an innate immune response of the organism is initiated, and an acquired immune system is finally activated, which plays an important role in specific and non-specific immune responses.

The TLRs play an important role in antiviral innate immunity. Through researches, it has found that the TLRs related to virus recognition and body antiviral immunity mainly included TLR2, TLR3, TLR4, TLR7, TLR8, and TLR9. The TLR2 and TLR4 on the surfaces of cells mainly recognize envelope protein of viruses; and the TLR3, TLR7, TLR8, and TLR9 in the cells mainly recognize viral nucleic acids.

TLR7/8 is one of TLR members, and is mainly distributed in the intracellular parts of plasmacytoid Dendritic Cell (pDC) and B cells; and the TLR7/8 mainly recognizes a ssRNA viruses, and plays an important role in recognition and clearance of pathogenic microorganisms in the human body. After “the PAMP” is recognized, the PAMP plays a role by starting signaling cascade; the PRRs are the key to the cascade reaction, and the TLRs 7 and 8 are important PRRs, and may stimulate antigen-presenting cells to induce DCs to secret various cytokines and express various costimulatory molecules, and stimulate the synthesis of interferon-α, Tumor Necrosis Factor (TNF), and interleukin (IL-1, IL-6, IL-8, and the like), so as to activate the innate immune response of the organism, and at the same time, the TLRs 7 and 8 may also activate pDC, improve anti-presentation capability of the pDC, promote the proliferation of CD4+T cells, and further activate the CD8+T cells, so as to kill tumor cells, thereby improving the antiviral and anti-tumor effect of the organism.

### SUMMARY

The disclosure provides a pharmaceutical combination and a use thereof in antitumor drugs. The pharmaceutical combination mainly consists of two parts:

(1) an immune checkpoint inhibitor (e.g., PD-1/PD-L1); (2) a Toll Like Receptor (TLR) agonist (such as an imidazole quinoline derivative). The pharmaceutical combination of the disclosure can be used to prepare efficient and low-toxicity anti-tumor drugs. By combining a PD-L1/PD-1 inhibitor with the TLR agonist, in an aspect, an inhibitory signal is relieved, the activation of T cells is improved, and an adaptive immune system response is promoted; and in another aspect, the expression of INF and cytokines is induced, aggregation of the T cells is promoted, and an innate immune system response is activated. In this way, cytotoxic cellular responses to tumors are improved by using a two-pronged approach, such that the pharmaceutical combination has high clinical prospects and disclosure values.

In one aspect, the disclosure provides a pharmaceutical combination including a programmed cell death protein 1 (PD-1) inhibitor and/or a programmed death ligand 1 (PD-L1) inhibitor, and a TLR agonist.

In some implementations, a TLR includes a TLR1, a TLR2, a TLR3, a TLR4, a TLR5, a TLR6, a TLR7, a TLR8, a TLR9, and/or a TLR10.

In some implementations, the TLR agonist is selected from one or more of a TLR7 agonist, a TLR8 agonist, or a TLR9 agonist.

In some implementations, the TLR agonist includes a TLR7 and TLR8 dual agonist (TLR7/TLR8 agonist).

In some implementations, the TLR agonist includes dsRNA, ssRNA, CpG DNA, an imidazole quinoline derivative, and/or a guanosine analogue.

In some implementations, the TLR agonist includes the imidazole quinoline derivative.

In some implementations, the TLR agonist is selected from one or more of Imiquimod, Gardiquimod, Resiquimod, 1V209, Selgantolimod (GS-9688), Vesatolimod (GS-9620), Sumanirole, PF-4878691, or derivatives and analogues thereof.

In some implementations, the TLR agonist includes the Imiquimod, Resiquimod, or pharmaceutically acceptable salts thereof.

In some implementations, the TLR agonist is selected from one or more of LHC-165, NKTR-262, DN1508052-01, SHR2150, CL307, CL264, Loxoribine, Isatoribine, DSR-6434, GSK2245035, SM-276001, SM-324405, SM-324406, AZ12441970, AZ12443988, or derivatives and analogues thereof.

In some embodiments, where the PD-1 inhibitor has one or more of the following characteristics:
a. inhibition or reduction of PD-1 expression, such as transcription or translation of PD-1;
b. inhibition or reduction of PD-1 activity, such as inhibition or reduction of PD-1 binding to its homologous ligands, such as PD-L1 or PD-L2; and
c. binding PD-1 or one or more of its ligands, such as PD-L1 or PD-L2.

In some embodiments, where the PD-1 inhibitor includes an anti PD-1 antibody or antigen-binding fragments thereof.

In some embodiments, where the anti PD-1 antibody is selected from the group consisting of Pembrolizumab, Nivolumab, Pidilizumab, Tislelizumab, Camrelizumab (SHR-1210), Sintilimab, Toripalimab, MEDI0680, BGB-A317, TSR-042, REGN2810, PF-06801591, RB0004, analogues thereof, or a combination thereof.

In some embodiments, where the anti PD-1 antibody includes at least one CDR in the antibody heavy chain variable region (VH), and the VH includes an amino acid sequence shown in SEQ ID NO: 8.

In some embodiments, where the anti PD-1 antibody includes a VH including HCDR3, and the HCDR3 includes an amino acid sequence shown in SEQ ID NO: 3.

In some embodiments, where the VH further includes HCDR2, wherein the HCDR2 includes an amino acid sequence shown in SEQ ID NO: 2.

In some embodiments, where the VH further includes HCDR1, wherein the HCDR 1 includes an amino acid sequence shown in SEQ ID NO: 1.

In some embodiments, where the VH includes HCDR1, HCDR2, and HCDR3, wherein the HCDR3 includes an amino acid sequence shown in SEQ ID NO: 3, the HCDR2 includes an amino acid sequence shown in SEQ ID NO: 2, and the HCDR1 includes an amino acid sequence shown in SEQ ID NO: 1.

In some embodiments, where the VH includes a framework region HFR1, the C-terminal of HFR1 is directly or indirectly connected to the N-terminal of HCDR1, and the HFR1 includes an amino acid sequence shown in SEQ ID NO: 4 or an amino acid sequence having at least about 70% sequence identity to the amino acid sequence shown in SEQ ID NO: 4.

In some embodiments, where the VH includes a framework region HFR2, the N-terminal of HFR2 is directly or indirectly connected to the C-terminal of HCDR1, and the C-terminal of HFR2 is directly or indirectly connected to the N-terminal of HCDR2; and the HFR2 includes an amino acid sequence shown in SEQ ID NO: 5 or an amino acid sequence having at least about 70% sequence identity to the amino acid sequence shown in SEQ ID NO: 5.

In some embodiments, where the VH includes a framework region HFR3, the N-terminal of HFR3 is directly or indirectly connected to the C-terminal of HCDR2, and the C-terminal of HFR3 is directly or indirectly connected to the N-terminal of HCDR3; and the HFR3 includes an amino acid sequence shown in SEQ ID NO: 6 or an amino acid sequence having at least about 70% sequence identity to the amino acid sequence shown in SEQ ID NO: 6.

In some embodiments, where the VH includes a framework region HFR4, the N-terminal of HFR4 is directly or indirectly connected to the C-terminal of HCDR3, and the HFR4 includes an amino acid sequence shown in SEQ ID NO: 7 or an amino acid sequence having at least about 70% sequence identity to the amino acid sequence shown in SEQ ID NO: 7.

In some embodiments, where the VH includes framework regions HFR1, HFR2, HFR3, and HFR4, the C-terminal of HFR1 is directly or indirectly connected to the N-terminal of HCDR1, the N-terminal of HFR2 is directly or indirectly connected to the C-terminal of HCDR1, and the C-terminal of HFR2 is directly or indirectly connected to the N-terminal of HCDR2, the N-terminal of HFR3 is directly or indirectly connected to the C-terminal of HCDR2, and the C-terminal of HFR3 is directly or indirectly connected to the N-terminal of HCDR3, the N-terminal of HFR4 is directly or indirectly connected to the C-terminal of HCDR3; among them, the HFR1 includes an amino acid sequence shown in SEQ ID NO: 4 or an amino acid sequence having at least about 70% sequence identity to the amino acid sequence shown in SEQ ID NO: 4, the HFR2 includes an amino acid sequence shown in SEQ ID NO: 5 or an amino acid sequence having at least about 70% sequence identity to the amino acid sequence shown in SEQ ID NO: 5, the HFR3 includes an amino acid sequence shown in SEQ ID NO: 6 or an amino acid sequence having at least about 70% sequence identity to the amino acid sequence shown in SEQ ID NO: 6, and the HFR4 includes an amino acid sequence shown in SEQ ID NO: 7 or an amino acid sequence having at least about 70% sequence identity to the amino acid sequence shown in SEQ ID NO: 7.

In some embodiments, where the anti PD-1 antibody includes a VH, and the VH includes an amino acid sequence shown in SEQ ID NO: 8.

In some embodiments, where the anti PD-1 antibody includes an antibody heavy chain (HC), and the HC includes an amino acid sequence shown in SEQ ID NO: 9.

In some embodiments, where the anti PD-1 antibody includes at least one CDR in the antibody light chain variable region (VL), and the VL includes an amino acid sequence shown in SEQ ID NO: 17.

In some embodiments, where the anti PD-1 antibody includes at least one CDR in VH, wherein the VH includes an amino acid sequence shown in SEQ ID NO: 8, and the anti PD-1 antibody includes at least one CDR in VL, and the VL includes an amino acid sequence shown in SEQ ID NO: 17.

In some embodiments, where the anti PD-1 antibody includes a VL including LCDR1, and the LCDR1 includes an amino acid sequence shown in SEQ ID NO: 10.

In some embodiments, where the VL further includes LCDR2, wherein the LCDR2 includes an amino acid sequence shown in SEQ ID NO: 11.

In some embodiments, where the VL further includes LCDR3, wherein the LCDR3 includes an amino acid sequence shown in SEQ ID NO: 12.

In some embodiments, where the VL includes LCDR1, LCDR2 and LCDR3, wherein the LCDR1 includes an amino acid sequence shown in SEQ ID NO: 10, the LCDR2 includes an amino acid sequence shown in SEQ ID NO: 11, and the LCDR3 includes an amino acid sequence shown in SEQ ID NO: 12.

In some embodiments, where the anti PD-1 antibody includes VH and antibody VL, the VH includes HCDR1, HCDR2, and HCDR3, wherein the HCDR3 includes an amino acid sequence shown in SEQ ID NO: 3, the HCDR2 includes an amino acid sequence shown in SEQ ID NO: 2, and the HCDR1 includes an amino acid sequence shown in SEQ ID NO: 1; and the VL includes LCDR1, LCDR2, and LCDR3, wherein the LCDR1 includes an amino acid sequence shown in SEQ ID NO: 10, the LCDR2 includes an amino acid sequence shown in SEQ ID NO: 11, and the LCDR3 includes an amino acid sequence shown in SEQ ID NO: 12.

In some embodiments, where the VL includes a framework region LFR1, the C-terminal of LFR1 is directly or indirectly connected to the N-terminal of LCDR1, and the LFR1 includes an amino acid sequence shown in SEQ ID NO: 13 or an amino acid sequence having at least about 70% sequence identity to the amino acid sequence shown in SEQ ID NO: 13.

In some embodiments, where the VL includes a framework region LFR2, the N-terminal of LFR2 is directly or indirectly connected to the C-terminal of LCDR1, and the C-terminal of LFR2 is directly or indirectly connected to the N-terminal of LCDR2; and the LFR2 includes an amino acid sequence shown in SEQ ID NO: 14 or an amino acid sequence having at least about 70% sequence identity to the amino acid sequence shown in SEQ ID NO: 14.

In some embodiments, where the VL includes a framework region LFR3, the N-terminal of LFR3 is directly or indirectly connected to the C-terminal of LCDR2, and the C-terminal of LFR3 is directly or indirectly connected to the N-terminal of LCDR3; and the LFR3 includes an amino acid sequence shown in SEQ ID NO: 15 or an amino acid sequence having at least about 70% sequence identity to the amino acid sequence shown in SEQ ID NO: 15.

In some embodiments, where the VL includes a framework region LFR4, the N-terminal of LFR4 is directly or indirectly connected to the C-terminal of LCDR3, and the LFR4 includes an amino acid sequence shown in SEQ ID NO: 16 or an amino acid sequence having at least about 70% sequence identity to the amino acid sequence shown in SEQ ID NO: 16.

In some embodiments, where the VL includes framework regions LFR1, LFR2, LFR3 and LFR4, the C-terminal of LFR1 is directly or indirectly connected to the N-terminal of LCDR1, the N-terminal of LFR2 is directly or indirectly connected to the C-terminal of LCDR1, and the C-terminal of LFR2 is directly or indirectly connected to the N-terminal of LCDR2, the N-terminal of LFR3 is directly or indirectly connected to the C-terminal of LCDR2, and the C-terminal of LFR3 is directly or indirectly connected to the N-terminal of LCDR3, the N-terminal of LFR4 is directly or indirectly connected to the C-terminal of LCDR3; among them, the LFR1 includes an amino acid sequence shown in SEQ ID NO: 13 or an amino acid sequence having at least about 70% sequence identity to the amino acid sequence shown in SEQ ID NO: 13, the LFR2 includes an amino acid sequence shown in SEQ ID NO: 14 or an amino acid sequence having at least about 70% sequence identity to the amino acid sequence shown in SEQ ID NO: 14, the LFR3 includes an amino acid sequence shown in SEQ ID NO: 15 or an amino acid sequence having at least about 70% sequence identity to the amino acid sequence shown in SEQ ID NO: 15, and the LFR4 includes an amino acid sequence shown in SEQ ID NO: 16 or an amino acid sequence having at least about 70% sequence identity to the amino acid sequence shown in SEQ ID NO:16.

In some embodiments, where the anti PD-1 antibody includes a VL, and the VL includes an amino acid sequence shown in SEQ ID NO: 17.

In some embodiments, where the anti PD-1 antibody includes VH and VL, the VH includes an amino acid sequence shown in SEQ ID NO: 8, and the VL includes an amino acid sequence shown in SEQ ID NO: 17.

In some embodiments, where the anti PD-1 antibody includes an antibody light chain (LC), and the LC includes an amino acid sequence shown in SEQ ID NO: 18.

In some embodiments, where the anti PD-1 antibody includes HC and LC, the HC includes an amino acid sequence shown in SEQ ID NO: 9, and the LC includes an amino acid sequence shown in SEQ ID NO: 18.

In some embodiments, where the PD-L1 inhibitor has one or more of the following characteristics:
a. inhibition or reduction of PD-L1 expression, such as transcription or translation of PD-L1;
b. inhibition or reduction of PD-L1 activity, such as inhibition or reduction of PD-L1 binding to its associated receptors such as PD-1; and
c. binding of PD-L1 or its receptors such as PD-1.

In some embodiments, where the PD-L1 inhibitor includes an anti PD-L1 antibody or antigen-binding fragments thereof.

In some embodiments, where the anti PD-L1 antibody is selected from the group consisting of Durvalumab, Atezolizumab, Avelumab, MDX-1105, YW243.55.S70, MDPL3280A, AMP-224, LY3300054, RB0005, analogues thereof, or a combination thereof.

In some embodiments, where the anti PD-L1 antibody includes at least one CDR in VH, the VH includes an amino acid sequence shown in SEQ ID NO: 25.

In some embodiments, where the anti PD-L1 antibody includes a VH, and the VH includes HCDR3, and the HCDR3 includes an amino acid sequence shown in SEQ ID NO: 21.

In some embodiments, where the VH further includes HCDR2, wherein the HCDR2 includes an amino acid sequence shown in SEQ ID NO: 20.

In some embodiments, where the VH further includes HCDR1, wherein the HCDR 1 includes an amino acid sequence shown in SEQ ID NO: 19.

In some embodiments, where the VH includes HCDR1, HCDR2, and HCDR3, wherein the HCDR3 includes an amino acid sequence shown in SEQ ID NO: 21, the HCDR2 includes an amino acid sequence shown in SEQ ID NO: 20, and the HCDR1 includes an amino acid sequence shown in SEQ ID NO: 19.

In some embodiments, where the VH includes a framework region HFR1, the C-terminal of HFR1 is directly or indirectly connected to the N-terminal of HCDR1, and the HFR1 includes an amino acid sequence shown in SEQ ID NO: 22 or an amino acid sequence having at least about 70% sequence identity to the amino acid sequence shown in SEQ ID NO: 22.

In some embodiments, where the VH includes a framework region HFR2, the N-terminal of HFR2 is directly or indirectly connected to the C-terminal of HCDR1, and the C-terminal of HFR2 is directly or indirectly connected to the N-terminal of HCDR2; and the HFR2 includes an amino acid sequence shown in SEQ ID NO: 23 or an amino acid sequence having at least about 70% sequence identity to the amino acid sequence shown in SEQ ID NO: 23.

In some embodiments, where the VH includes a framework region HFR3, the N-terminal of HFR3 is directly or indirectly connected to the C-terminal of HCDR2, and the C-terminal of HFR3 is directly or indirectly connected to the N-terminal of HCDR3; and the HFR3 includes an amino acid sequence shown in SEQ ID NO: 24 or an amino acid sequence having at least about 70% sequence identity to the amino acid sequence shown in SEQ ID NO: 24.

In some embodiments, where the VH includes a framework region HFR4, the N-terminal of HFR4 is directly or indirectly connected to the C-terminal of HCDR3, and the HFR4 includes an amino acid sequence shown in SEQ ID NO: 7 or an amino acid sequence having at least about 70% sequence identity to the amino acid sequence shown in SEQ ID NO: 7.

In some embodiments, where the VH includes framework regions HFR1, HFR2, HFR3, and HFR4, the C-terminal of HFR1 is directly or indirectly connected to the N-terminal of HCDR1, the N-terminal of HFR2 is directly or indirectly connected to the C-terminal of HCDR1, and the C-terminal of HFR2 is directly or indirectly connected to the N-terminal of HCDR2, the N-terminal of HFR3 is directly or indirectly connected to the C-terminal of HCDR2, and the C-terminal of HFR3 is directly or indirectly connected to the N-terminal of HCDR3, the N-terminal of HFR4 is directly or indirectly connected to the C-terminal of HCDR3; among them, the HFR1 includes an amino acid sequence shown in SEQ ID NO: 22 or an amino acid sequence having at least about 70% sequence identity to the amino acid sequence shown in SEQ ID NO: 22, the HFR2 includes an amino acid sequence shown in SEQ ID NO: 23 or an amino acid sequence having at least about 70% sequence identity to the amino acid sequence shown in SEQ ID NO: 23, the HFR3 includes an amino acid sequence shown in SEQ ID NO: 24 or an amino acid sequence having at least about 70% sequence identity to the amino acid sequence shown in SEQ ID NO: 24, and the HFR4 includes an amino acid sequence shown in SEQ ID NO: 7 or an amino acid sequence having at least about 70% sequence identity to the amino acid sequence shown in SEQ ID NO: 7.

In some embodiments, where the anti PD-L1 antibody includes VH, and the VH includes an amino acid sequence shown in SEQ ID NO: 25.

In some embodiments, where the anti PD-L1 antibody includes HC, and the HC includes an amino acid sequence shown in SEQ ID NO: 26.

In some embodiments, where the anti PD-L1 antibody includes at least one CDR in VL, and the VL includes an amino acid sequence shown in SEQ ID NO: 37.

In some embodiments, where the anti PD-L1 antibody includes at least one CDR in VH, the VH includes an amino acid sequence shown in SEQ ID NO: 25, and the anti PD-L1 antibody includes at least one CDR in VL, and the VL includes an amino acid sequence shown in SEQ ID NO: 37.

In some embodiments, where the anti PD-L1 antibody includes at least one CDR in VH, the VH includes an amino acid sequence shown in SEQ ID NO: 25, and the anti PD-L1 antibody includes at least one CDR in VL, and the VL includes an amino acid sequence shown in SEQ ID NO:38, SEQ ID NO: 39 or SEQ ID NO:40.

In some embodiments, where the anti PD-L1 antibody includes a VL including LCDR1, and the LCDR1 includes an amino acid sequence shown in SEQ ID NO: 27.

In some embodiments, where the anti PD-L1 antibody includes a VL including LCDR1, and the LCDR1 includes an amino acid sequence shown in SEQ ID NO:28, SEQ ID NO:29 or SEQ ID NO: 30.

In some embodiments, where the VL further includes LCDR2, wherein the LCDR2 includes an amino acid sequence shown in SEQ ID NO: 31.

In some embodiments, where the VL further includes LCDR3, wherein the LCDR3 includes an amino acid sequence shown in SEQ ID NO: 32.

In some embodiments, where the VL includes LCDR1, LCDR2 and LCDR3, wherein the LCDR1 includes an amino acid sequence shown in SEQ ID NO: 27, the LCDR2 includes an amino acid sequence shown in SEQ ID NO: 31, and the LCDR3 includes an amino acid sequence shown in SEQ ID NO: 32.

In some embodiments, where the VL includes LCDR1, LCDR2 and LCDR3, wherein the LCDR1 includes an amino acid sequence shown in SEQ ID NO: 28, the LCDR2 includes an amino acid sequence shown in SEQ ID NO: 31, and the LCDR3 includes an amino acid sequence shown in SEQ ID NO: 32;
the LCDR1 includes an amino acid sequence shown in SEQ ID NO: 29, the LCDR2 includes an amino acid sequence shown in SEQ ID NO: 31, and the LCDR3 includes an amino acid sequence shown in SEQ ID NO: 32; or
the LCDR1 includes an amino acid sequence shown in SEQ ID NO: 30, the LCDR2 includes an amino acid sequence shown in SEQ ID NO: 31, and the LCDR3 includes an amino acid sequence shown in SEQ ID NO: 32.

In some embodiments, where the anti PD-L1 antibody includes VH and antibody VL, the VH includes HCDR1, HCDR2, and HCDR3, wherein the HCDR3 includes an amino acid sequence shown in SEQ ID NO: 21, the HCDR2 includes an amino acid sequence shown in SEQ ID NO: 20, and the HCDR1 includes an amino acid sequence shown in SEQ ID NO: 19; and the VL includes LCDR1, LCDR2, and LCDR3, wherein the LCDR1 includes an amino acid sequence shown in SEQ ID NO: 27, the LCDR2 includes an amino acid sequence shown in SEQ ID NO: 31, and the LCDR3 includes an amino acid sequence shown in SEQ ID NO: 32.

In some embodiments, where the anti PD-L1 antibody includes VH and antibody VL, the VH includes HCDR1, HCDR2, and HCDR3, wherein the HCDR3 includes an amino acid sequence shown in SEQ ID NO: 21, the HCDR2 includes an amino acid sequence shown in SEQ ID NO: 20, and the HCDR1 includes an amino acid sequence shown in SEQ ID NO: 19; and the VL includes LCDR1, LCDR2, and LCDR3, wherein the LCDR1 includes an amino acid sequence shown in SEQ ID NO: 28, SEQ ID NO:29 or SEQ ID NO:30, the LCDR2 includes an amino acid sequence shown in SEQ ID NO: 31, and the LCDR3 includes an amino acid sequence shown in SEQ ID NO: 32.

In some embodiments, where the VL includes a framework region LFR1, the C-terminal of LFR1 is directly or indirectly connected to the N-terminal of LCDR1, and the LFR1 includes an amino acid sequence shown in SEQ ID NO: 33 or an amino acid sequence having at least about 70% sequence identity to the amino acid sequence shown in SEQ ID NO: 33.

In some embodiments, where the VL includes a framework region LFR2, the N-terminal of LFR2 is directly or indirectly connected to the C-terminal of LCDR1, and the C-terminal of LFR2 is directly or indirectly connected to the N-terminal of LCDR2; and the LFR2 includes an amino acid sequence shown in SEQ ID NO: 34 or an amino acid sequence having at least about 70% sequence identity to the amino acid sequence shown in SEQ ID NO: 34.

In some embodiments, where the VL includes a framework region LFR3, the N-terminal of LFR3 is directly or indirectly connected to the C-terminal of LCDR2, and the C-terminal of LFR3 is directly or indirectly connected to the N-terminal of LCDR3; and the LFR3 includes an amino acid sequence shown in SEQ ID NO: 35 or an amino acid sequence having at least about 70% sequence identity to the amino acid sequence shown in SEQ ID NO: 35.

In some embodiments, where the VL includes a framework region LFR4, the N-terminal of LFR4 is directly or indirectly connected to the C-terminal of LCDR3, and the LFR4 includes an amino acid sequence shown in SEQ ID NO: 36 or an amino acid sequence having at least about 70% sequence identity to the amino acid sequence shown in SEQ ID NO: 36.

In some embodiments, where the VL includes framework regions LFR1, LFR2, LFR3 and LFR4, the C-terminal of LFR1 is directly or indirectly connected to the N-terminal of LCDR1, the N-terminal of LFR2 is directly or indirectly connected to the C-terminal of LCDR1, and the C-terminal of LFR2 is directly or indirectly connected to the N-terminal of LCDR2, the N-terminal of LFR3 is directly or indirectly connected to the C-terminal of LCDR2, and the C-terminal of LFR3 is directly or indirectly connected to the N-terminal of LCDR3, the N-terminal of LFR4 is directly or indirectly connected to the C-terminal of LCDR3; among them, the LFR1 includes an amino acid sequence shown in SEQ ID NO: 33 or an amino acid sequence having at least about 70% sequence identity to the amino acid sequence shown in SEQ ID NO: 33, the LFR2 includes an amino acid sequence shown in SEQ ID NO: 34 or an amino acid sequence having at least about 70% sequence identity to the amino acid sequence shown in SEQ ID NO: 34, the LFR3 includes an amino acid sequence shown in SEQ ID NO: 35 or an amino acid sequence having at least about 70% sequence identity to the amino acid sequence shown in SEQ ID NO: 35, and the LFR4 includes an amino acid sequence shown in SEQ ID NO: 36 or an amino acid sequence having at least about 70% sequence identity to the amino acid sequence shown in SEQ ID NO:36.

In some embodiments, where the anti PD-L1 antibody includes a VL, and the VL includes an amino acid sequence shown in SEQ ID NO: 37.

In some embodiments, where the anti PD-L1 antibody includes a VL, and the VL includes an amino acid sequence shown in SEQ ID NO:38, SEQ ID NO:39 or SEQ ID NO:40.

In some embodiments, where the anti PD-L1 antibody includes VH and VL, the VH includes an amino acid sequence shown in SEQ ID NO: 25, and the VL includes an amino acid sequence shown in SEQ ID NO: 37.

In some embodiments, where the anti PD-L1 antibody includes VH and VL, the VH includes an amino acid sequence shown in SEQ ID NO: 25, and the VL includes an amino acid sequence shown in SEQ ID NO: 38, SEQ ID NO:39 or SEQ ID NO:40.

In some embodiments, where the anti PD-L1 antibody includes LC, and the LC includes an amino acid sequence shown in SEQ ID NO: 41.

In some embodiments, where the anti PD-L1 antibody includes LC, and the LC includes an amino acid sequence shown in SEQ ID NO:42, SEQ ID NO:43 or SEQ ID NO:44.

In some embodiments, where the anti PD-L1 antibody includes HC and LC, the HC includes an amino acid sequence shown in SEQ ID NO: 26, and the LC includes an amino acid sequence shown in SEQ ID NO: 41.

In some embodiments, where the anti PD-L1 antibody includes HC and LC, the HC includes an amino acid sequence shown in SEQ ID NO: 26, and the LC includes an amino acid sequence shown in SEQ ID NO:42, SEQ ID NO:43 or SEQ ID NO:44.

In some embodiments, i) the PD-1 inhibitor and/or PD-L1 inhibitor in the pharmaceutical combination are not mixed with ii) the TLR agonist to each other in the pharmaceutical combination.

In some embodiments, i) the PD-1 inhibitor and/or PD-L1 inhibitor, and ii) the TLR agonist are present in the pharmaceutical combination in a single dosage form.

In some embodiments, where the pharmaceutical combination is formulated into a pharmaceutical composition.

In some embodiments, where the pharmaceutical composition includes a PD-1 inhibitor or a PD-L1 inhibitor, and a TLR agonist.

In some embodiments, where the TLR agonist is present in an amount of about 0.0001 mg/kg to about 200 mg/kg.

In some embodiments, where the PD-1 inhibitor or PD-L1 inhibitor is present in an amount of 0.0001 mg/kg to about 200 mg/kg.

In some embodiments, where the pharmaceutical composition further includes one or more pharmaceutically acceptable carriers.

In another aspect, the disclosure also provides a use of the aforementioned pharmaceutical combination in the preparation of drugs for the treatment of neoplastic diseases.

In some embodiments, where the neoplastic diseases include tumors and/or wart diseases.

In another aspect, the disclosure also provides the aforementioned pharmaceutical combination for use in the treatment of neoplastic diseases.

In another aspect, the disclosure also provides a drug including the aforementioned pharmaceutical combination for treating neoplastic diseases.

In another aspect, the disclosure also provides a method for treating neoplastic diseases, which includes administering an effective amount of the aforementioned pharmaceutical combination to a subject in need thereof.

In some embodiments, where the subject suffers from neoplasm.

In some embodiments, where the neoplasm includes tumors and/or warts.

In some embodiments, where the administration includes local, intraneoplastic (e.g., in tumors or warts), or systemic administrations.

In some embodiments, where the administration includes intravenous injection, intravenous instillation, intramuscular injection, subcutaneous injection, and/or intraneoplastic injection.

In some embodiments, i) the PD-1 inhibitor or PD-L1 inhibitor, and ii) the TLR agonist in the pharmaceutical combination are administered by use of the same or different administration routes. In some embodiments, it includes injection of the TLR agonist into the neoplasm.

In some embodiments, it also includes injection or systemic infusion of the PD-1 inhibitor or PD-L1 inhibitor into the neoplasm.

In some embodiments, it includes injection of i) the PD-1 inhibitor or PD-L1 inhibitor TLR agonist, and ii) the TLR agonist in the pharmaceutical combination into the neoplasm.

In some embodiments, where i) the PD-1 inhibitor or PD-L1 inhibitor, and ii) the TLR agonist in the pharmaceutical combination are administered simultaneously or at different times.

In some embodiments, where the PD-1 inhibitor or PD-L1 inhibitor is administered before and/or after the administration of the TLR agonist.

In some embodiments, where the PD-1 inhibitor or PD-L1 inhibitor is administered after the administration of the TLR agonist.

In some embodiments, the method includes: i) injecting the TLR agonist into the neoplasm; and ii) injection or systemic infusion of the PD-1 inhibitor or the PD-L1 inhibitor into the neoplasm or the whole body after the TLR agonist is administered.

In some embodiments, the method includes: i) injection of the TLR agonist into the neoplasm; ii) injection or systemic infusion of the PD-1 inhibitor or PD-L1 inhibitor into the neoplasm after administering the TLR agonist.

In some implementations, the PD-1 inhibitor or the PD-L1 inhibitor is administered about 2h to about 72h after the TLR agonist is administered.

In some implementations, the PD-1 inhibitor or the PD-L1 inhibitor is administered about 2 h, about 4 h, about 8 h, about 16 h, about 24 h, about 3 6h, about 48 h, about 60 h, or about 72 h after the TLR agonist is administered.

In some implementations, the method includes: i) injecting the TLR agonist into the neoplasm; and ii) injecting or infusing the PD-1 inhibitor or the PD-L1 inhibitor into the neoplasm or the whole body about 48h after the TLR agonist is administered.

In some embodiments, where i) the PD-1 inhibitor or PD-L1 inhibitor, and ii) the TLR agonist in the pharmaceutical combination are administered simultaneously.

In some embodiments, where i) the PD-1 inhibitor or PD-L1 inhibitor, and ii) the TLR agonist in the pharmaceutical combination are simultaneously administered by way of intraneoplastic injection, and the PD-1 inhibitor or PD-L1 inhibitor and the TLR agonist are present in a same dosage form.

In some implementations, i) the PD-1 inhibitor or the PD-L1 inhibitor in the pharmaceutical combination, and ii) the TLR agonist are administered at the same time by means of intra-neoplasm injection; and the PD-1 inhibitor or the PD-L1 inhibitor and the TLR agonist are located in separate dosage forms.

In another aspect, the disclosure provides a pharmaceutical kit including the aforementioned pharmaceutical combination.

Those skilled in the art can have an insight into other aspects and advantages of the disclosure from the detailed description below. The detailed description below only shows and describes exemplary embodiments of the disclosure. As those skilled in the art will recognize, the content of the disclosure enables those skilled in the art to make modifications to the disclosed specific embodiments without departing from the spirit and scope of the invention involved in the disclosure. Correspondingly, the accompanying drawings and the descriptions in the specification of the disclosure are only illustrative, rather than being restrictive.

### BRIEF DESCRIPTION OF THE DRAWINGS

The specific features of the invention involved in the disclosure are shown in the attached claims. The characteristics and advantages of the invention involved in the disclosure can be better understood by referring to the exemplary embodiments and accompanying drawings described in detail hereinafter. A brief explanation of the attached drawings is as follows:

FIG. 1 shows the in vivo efficacy study of the pharmaceutical combination of PD-L1 inhibitor RB0005 and Imiquimod according to the disclosure, as shown by changes in body weight of each group of mice during administration.

FIG. 2 shows the in vivo efficacy study of the pharmaceutical combination of PD-L1 inhibitor RB0005 and Imiquimod according to the disclosure, as shown by changes in survival rate of each group of mice during administration.

FIG. 3A shows the in vivo efficacy study of the pharmaceutical combination of PD-L1 inhibitor RB0005 and Imiquimod according to the disclosure, as shown by changes in tumor proliferation of each group of mice during administration.

FIG. 3B shows the in vivo efficacy study of the pharmaceutical combination of PD-L1 inhibitor RB0005 and Imiquimod according to the disclosure, as shown by changes in tumor proliferation of each group of mice on day 15 after treatment.

FIG. 4 shows changes in survival rates of mice of each group during in vivo drug efficacy study-administration of a pharmaceutical combination of a PD-1 inhibitor RB0004 and Imiquimod according to the present disclosure.

FIG. 4 shows the in vivo efficacy study of the pharmaceutical combination of PD-L1 inhibitor RB0004 and Imiquimod according to the disclosure, as shown by changes in survival rates of mice of each group.

FIG. 5A shows the in vivo efficacy study of the pharmaceutical combination of PD-1 inhibitor RB0004 and Imiquimod according to the disclosure, as shown by changes in tumor proliferation of each group of mice during administration.

FIG. 5B shows the in vivo efficacy study of the pharmaceutical combination of PD-L1 inhibitor RB0004 and Imiquimod according to the disclosure, as shown by changes in tumor proliferation of each group of mice on day 15 after treatment.

FIG. 6 shows the in vivo efficacy study of the pharmaceutical combination of PD-1 inhibitor RB0004 and Imiquimod according to the disclosure, as shown by tumor weight inhibitory rate of mice obtained through dissection after the administration and observation have completed.

FIG. 7 shows the in vivo efficacy study of the pharmaceutical combination of PD-L1 inhibitor RB0005 and different doses of Imiquimod according to the disclosure, as shown by changes in survival rate of each group of mice during administration.

FIG. 8 shows the in vivo efficacy study of the pharmaceutical combination of PD-L1 inhibitor RB0005 and different doses of Imiquimod according to the disclosure, as shown by tumor weight inhibitory rate of mice obtained through dissection after the administration and observation have completed.

FIG. 9 shows the in vivo efficacy study of the pharmaceutical combination of PD-L1 inhibitor RB0005 and different doses of Imiquimod according to the disclosure, as shown by changes in body weight of each group of mice during administration.

FIG. 10 shows the in vivo efficacy study of the pharmaceutical combination of PD-L1 inhibitor RB0005 and different doses of Imiquimod (including 5% of Imiquimod cream) according to the disclosure, as shown by changes in survival rate of each group of mice during administration.

FIG. 11 shows the in vivo efficacy study of the pharmaceutical combination of PD-L1 inhibitor RB0005 and different doses of Imiquimod (including 5% of Imiquimod cream) according to the disclosure, as shown by tumor weight inhibitory rate of mice obtained through dissection after the administration and observation have completed.

FIG. 12 shows the in vivo efficacy study of the pharmaceutical combination of PD-1 inhibitor RB0004 and different doses of Imiquimod (including 5% of Imiquimod cream) according to the disclosure, as shown by changes in survival rate of each group of mice during administration.

FIG. 13 shows the in vivo efficacy study of the pharmaceutical combination of PD-1 inhibitor RB0004 and different doses of Imiquimod (including 5% of Imiquimod cream) according to the disclosure, as shown by tumor weight inhibitory rate of mice obtained through dissection after the administration and observation have completed.

FIG. 14 shows the trend of tumor proliferation in mice of each group during administration according to Example 6 of the present disclosure.

FIG. 15 shows survival curves of mice of each group during administration according to Example 6 of the present disclosure.

FIG. 16 shows average tumor mass of mice of each group during administration according to Example 6 of the present disclosure.

FIG. 17 shows the trend of tumor proliferation in mice of each group during administration according to Example 7 of the present disclosure.

FIG. 18 shows survival curves of mice of each group during administration according to Example 7 of the present disclosure.

FIG. 19 shows average tumor mass of mice of each group during administration according to Example 7 of the present disclosure.

FIG. 20 shows the trend of tumor proliferation in mice of each group during administration according to Example 8 of the present disclosure.

FIG. 21 shows survival curves of mice of each group during administration according to Example 8 of the present disclosure.

FIG. 22 shows average tumor mass of mice of each group during administration according to Example 8 of the present disclosure.

FIG. 23 shows the trend of tumor proliferation in mice of each group during administration according to Example 9 of the present disclosure.

FIG. 24 shows survival curves of mice of each group during administration according to Example 9 of the present disclosure.

FIG. 25 shows average tumor mass of mice of each group during administration according to Example 9 of the present disclosure.

FIG. 26 shows the trend of tumor proliferation in each group of mice during administration in Example 10.1 of the disclosure.

FIG. 27 shows the survival curve of each group of mice during administration in Example 10.1 of the disclosure.

FIG. 28 shows the mean tumor mass of each group of mice during administration in Example 10.1 of the disclosure.

FIG. 29 shows the trend of tumor proliferation in each group of mice during administration in Example 10.2 of the disclosure.

FIG. 30 shows the survival curve of each group of mice during administration in Example 10.2 of the disclosure.

FIG. 31 shows the mean tumor mass of each group of mice during administration in Example 10.2 of the disclosure.

FIG. 32 shows the trend of changes in weights of mice of each group during administration according to Example 11 of the present disclosure.

FIG. 33 shows the trend of tumor proliferation in mice of each group during administration according to Example 11 of the present disclosure.

FIG. 34 shows average tumor mass of mice of each group during administration according to Example 11 of the present disclosure.

FIG. 35 is a schematic diagram of sequential administration according to Example 12 of the present disclosure.

FIG. 36 shows the trend of tumor proliferation in mice of each group during administration according to Example 12 of the present disclosure.

FIG. 37 shows survival curves of mice of each group during administration according to Example 12 of the present disclosure.

FIG. 38 shows average tumor mass of mice of each group during administration according to Example 12 of the present disclosure.

### DETAILED DESCRIPTION OF THE EMBODIMENTS

The embodiments of the present invention will be illustrated below by way of specific examples, and other advantages and effects of the present disclosure can be easily understood by those familiar with this technology from the content disclosed in this specification.

### Definitions of Terms

In the disclosure, the term “PD-1” generally refers to programmed cell death protein 1, i.e. a type I membrane protein of 288 amino acids first described in 1992 (Ishida et al., EMBO J., 11 (1992), 3887-3895). PD-1 is a member of the expanded CD28/CTLA-4T cell regulator family and has two ligands, i.e. PD-L1 (B7-H1 and CD274) and PD-L2 (B7-DC and CD273). The structure of this protein includes an extracellular IgV domain, followed by a transmembrane region and an intracellular tail. The intracellular tail contains two phosphorylation sites located in the immunoreceptor tyrosine-based inhibitory motif and the immunoreceptor tyrosine-based switch motif, indicating a negative regulation of TCR signaling by PD-1. This is consistent with the binding of SHP-1 and SHP-2 phosphatases to the cytoplasmic tail of PD-1 after ligand binding. Although PD-1 is not expressed on naive T cells, it is upregulated after the T cell receptor (TCR) mediated activation and observed on both activated and depleted T cells (Agata et al., Int. Immunology 8 (1996), 765-772). These depleted T cells exhibit a dysfunctional phenotype and are unable to respond appropriately. Although PD-1 has a relatively broad expression pattern, its most important role is likely to act as a co-inhibitory receptor on T cells (Chinai et al., Trends in Pharmacological Sciences 36 (2015), 587-595). The current treatment approaches thus focus on blocking the interaction between PD-1 and its ligand to enhance T cell response. In this disclosure, the PD-1 may include human PD-1 (hPD-1), or variants, isotypes, and species homologs thereof, as well as analogues having at least one common epitope with hPD-1. The amino acid sequence of exemplary hPD-1 can be found under GenBank accession No.: U64863.

In this disclosure, the term “PD-L1” generally refers to programmed cell death 1 ligand 1, which can also be referred to as B7 homolog 1, B7-H1, differentiation cluster 274, (3) 274, or CD274, which, when combined with PD-1, downregulates T cell activation and cytokine secretion. “PD-L1” includes any natural PD-L1 from any vertebrate sources, including mammals such as primates (e.g., humans and Macaca fascicularis) and rodents (e.g., mice and rats). The term contemplates “full length”, unprocessed PD-L1, and any form of PD-L1 produced by cell processing. PD-L1 can exist as a transmembrane protein or as a soluble protein. “PD-L1” includes the full PD-L1 and fragments thereof, as well as functional variants, isoforms, species homologs, derivatives, analogues of PD-L1, and analogues having at least one common epitope with PD-L1. The basic structure of PD-L1 includes four domains, i.e. extracellular Ig like V-type domain and Ig like C2-type domain, transmembrane domain, and cytoplasmic domain. An exemplary human PD-L1 amino acid sequence can be found under the NCBI accession No.: NP_001254653 or UniProt accession No.: Q9NZQ7.

In this disclosure, the term “inhibitor” generally refers to a compound/substance or composition that can completely or partially prevent or reduce the physiological functions of one or more specific biomolecules (e.g., proteins (such as PD-1 or PD-L1), polypeptides, lipopolysaccharides, glycoproteins, ribonucleoprotein complexes, etc.). The reduce of physiological functions of one or more specific proteins can include a decrease in the activity of the protein itself (e.g., its ability to bind to other molecules, etc.) or a decrease in the existence quantity of the protein itself. Suitable inhibitor molecules can include antagonist antibodies or antibody fragments, small molecular fragments or derivatives, peptides, antisense oligonucleotides, small organic molecules, etc. In some embodiments, the inhibitor can block the activation of cellular signaling pathways. In some embodiments, the PD-1/PD-L1 inhibitor is an anti PD-1/PD-L1 antibody or antigen-binding fragments thereof.

In the present disclosure, the terms “Pembrolizumab”, “Nivolumab”, “Pidilizumab”, “Tislelizumab”, “Camrelizumab (SHR-1210)”, “Sintilimab”, “Toripalimab”, “MEDI0680”, “BGB-A317”, “TSR-042”, “REGN2810”, “PF-06801591”, “Durvalumab”, “Atezolizumab”, “Avelumab”, “MDX-1105”, “YW243.55.S70”, “MDPL3280A”, “AMP-224”, “LY3300054”, “RB0004”, and “RB0005” are used according to their ordinary and common meaning as understood in the art.

In the present disclosure, the term “Toll Like Receptor” and TLR generally refer to any member of a highly-conserved mammalian protein family that recognizes a pathogen-associated molecular pattern and acts as a key signal transduction element in innate immunity. TLR polypeptides share characteristic structures, which include an extracellular structural domain rich in leucine repeats, a transmembrane structural domain, and an intracellular structural domain participating in TLR signal transduction.

In the present disclosure, the term “Toll Like Receptor 7” and “TLR7” generally refer to a nucleic acid or polypeptide that shares at least 70%, 80%, 90%, 95%, 96%, 97%, 98%, 99% or more homology to a publicly available TLR7 sequence, such as a human TLR7 polypeptide GenBank login number AAZ99026, or murine TLR7 polypeptide GenBank login number AAK62676.

In the present disclosure, the term “Toll Like Receptor 8” and “TLR8” generally refer to a nucleic acid or polypeptide that shares at least 70%, 80%, 90%, 95%, 96%, 97%, 98%, 99% or more homology to a publicly available TLR8 sequence, such as a human TLR8 polypeptide GenBank login number AAZ95441, or murine TLR8 polypeptide GenBank login number AAK62677.

In the present disclosure, the term “Toll Like Receptor 9” and “TLR9” (also known as CD289, UNQ5798, or PRO19605) generally refer to a nucleotide sensitive TLR that is activated by unmethylated Cytidine-Phosphate-Guanosine (CpG) dinucleotide. An example of the TLR9 includes, but is not limited to a human TLR9, which is a protein with 1032-amino-acid-long that is coded by a mRNA transcript (NM_017442.3) with 3922-nucleotide-long. An amino acid sequence of the illustrated human TLR9 is expressed with a GenBank login number NP_059138.1. In the present disclosure, the term “TLR9” includes species other than human, such as a TLR9 homolog of Macaca Fascicularis (cynomolgus monkey) or Pantroglodytes (chimpanzee). The term “TLR9” includes proteins containing mutations, such as point mutations, fragments, insertions, deletions and splice variants, of a full-length wild-type TLR9. The term “TLR9” also encompasses post-translational modification of the amino acid sequence of the TLR9.

In the present disclosure, the term “agonist” generally refers to molecules (i.e., a regulator) that directly or indirectly regulate other molecules (for example, the TLR) and increase the activity, activation, or functions of other molecules. The agonist may include proteins, nucleic acids, sugars, organic molecules, small organic molecules (with or without organic parts) or other molecules. For example, a regulator improving gene transcription of the proteins, biological activity or biochemical functions is a substance that improves the transcription of the proteins or stimulates biochemical properties or activity of the proteins. For example, the agonist may induce, stimulate, increase, activate, promote, improve, or up-regulate the activity of a receptor, and such activity is known as “agonist activity”.

In the present disclosure, a “TLR agonist” is a substance that directly or indirectly binds the TLR (for example, the TLR7 and/or the TLR8) to induce TLR signal transduction. Any detectable difference in TLR signal transduction may indicate agonist stimulation or activation of the TLR. The signal transduction difference may manifested, for example, by changes in expression of target genes, changes in phosphorylation of signal transduction components, changes in intracellular localization of downstream elements such as a nuclear factor-KB (NF-KB), changes in association of certain components (such as IL-1 Receptor-Associated Kinase (IRAK)) with other proteins or intracellular structures, or changes in biochemical activity of components, such as kinases (for example, Mitogen-Activated Protein Kinase (MAPK)). In the present disclosure, the term “TLR agonist” refers to any compound that serves as the agonist of the TLR.

In the present disclosure, the term “derivative” generally refers to a chemical substance that is structurally related to another chemical substance, or may be a chemical substance that may be prepared by another chemical substance (i.e., a chemical substance derived from the chemical substance), for example, by means of chemical or enzymatic modifications. Derivative of organic molecules includes, but is not limited to, modified molecules, for example, molecules that are modified by adding or deleting hydroxyl, methyl, ethyl, carboxyl, nitro or amino. For example, the organic molecules may also be esterified, alkylated, and/or phosphorylated.

In the present disclosure, the term “pharmaceutically acceptable derivative” includes an isomer, a salt, an ester, an enol ether, an enol ester, an acetal, a ketal, an ortho ester, a hemiacetal, a hemiketal, an acid, a base, a solvate, a hydrate, or a prodrug thereof. Such derivatives may be readily prepared by those skilled in the art using known methods for such derivatization.

In the present disclosure, the term “isomer” generally refers to different compounds that have the same molecular formula but differ in atomic arrangement and configuration. The term “isomer” includes, but is not limited to, an optical isomer and analogues, a structural isomer and analogues, and a conformational isomer and analogues, etc.

In the present disclosure, the term “analogue” generally refers to its recognized in the art. In the context of non-protein analogues, the term “analogue” generally refers to a second organic or inorganic molecule that has a similar or identical function to a first organic or inorganic molecule and is structurally similar to the first organic or inorganic molecule. When the polypeptides or proteins are mentioned, the term “analogue” generally refers to a modified peptide or protein in which one or more amino acid residues of the peptide/protein have been replaced with other amino acid residues and/or in which one or more amino acid residues have been removed from the peptide/protein and/or in which one or more amino acid residues have been added to the peptide/protein.

In the present disclosure, the term “pharmaceutically acceptable salt” generally refers to a pharmaceutically acceptable organic or inorganic salt of a compound. The exemplary salt includes, but is not limited to, sulfate, citrate, acetate, oxalate, chloride, bromide, iodide, nitrate, bisulfate, phosphate, acid phosphate, isonicotinic acid salt, lactate, salicylate, acid citrate, tartrate, oleate, tannate, pantothenate, bitartrate, ascorbate, succinate, maleate, gentisate, fumarate, gluconate, glucuronic acid salt, saccharate, formate, benzoate, glutamate, mesylate, esylate, benzene sulfonate, p-toluenesulfonate, pamoate (1, 1′-methylene-bis-(2-hydroxy-3-naphthoic acid) salt), alkali metal (such as sodium and potassium) salt, alkaline earth metal (such as magnesium) salt, and ammonium salt. The pharmaceutically acceptable salt may involve the inclusion of another molecule, and the molecule includes, for example, an acetate ion, a succinate ion, or other counter ions. The counter ion may be any organic or inorganic part, which stabilizes the charge on a parent compound. In addition, the structure of the pharmaceutically acceptable salt may have more than one charged atom. In an example that a plurality of charged atoms are part of the pharmaceutically acceptable salt, the salt may have a plurality of counter ions. Therefore, the pharmaceutically acceptable salt may have one or more charged atoms and/or one or more counter ions.

In this disclosure, the term “antibody” generally refers to one being used in the broadest sense and specifically covering monoclonal antibodies, polyclonal antibodies, dimers, multimers, multispecific antibodies (e.g., bispecific antibodies), and antibody fragments, as long as they exhibit the desired biological activities (Miller et al. (2003) Jour. of Immunology 170:4854-4861). Antibodies can be mouse, human, humanized, chimeric antibodies, or those derived from other species.

A full-length antibody typically refers to an antibody composed of two “full-length antibody heavy chains” and two “full-length antibody light chains”. The “full length antibody heavy chain” is generally such a polypeptide that is composed of the antibody heavy chain variable domain (VH), antibody constant heavy chain domain 1 (CH1), antibody hinge region (HR), antibody heavy chain constant domain 2 (CH2), and antibody heavy chain constant domain 3 (CH3) in the direction of N-terminal to C-terminal, abbreviated as VH-CH1-HR-CH2-CH3; and in the case of IgE subtype antibodies, optionally further including the antibody heavy chain constant domain 4 (CH4). In some embodiments, the “full length antibody heavy chain” is a polypeptide composed of VH, CH1, HR, CH2, and CH3 in the direction of N-terminal to C-terminal. The “full length antibody light chain” is generally a polypeptide composed of an antibody light chain variable domain (VL) and an antibody light chain constant domain (CL) in the direction of N-terminal to C-terminal, abbreviated as VL-CL. The antibody light chain constant domain (CL) can be κ (kappa) or λ (lambda). Two full-length antibody chains are linked together by an interpolypeptide disulfide bond between the CL domain and the CH1 domain, and an interpolypeptide disulfide bond between the hinge regions of the full-length antibody heavy chain. Typical examples of full-length antibodies are natural antibodies such as IgG (e.g., IgG1 and IgG2), IgM, IgA, IgD, and IgE).

In this disclosure, the term “antigen binding fragment” generally refers to a portion of the antibody molecule that contains amino acids responsible for the specific binding between the antibody and the antigen. The part of an antigen that is specifically recognized and bound by an antibody is called as an “epitope” as mentioned above. The antigen binding domain can typically include the antibody light chain variable region (VL) and the antibody heavy chain variable region (VH); however, it does not necessarily include both. Fd fragments for example have two VH regions, and generally retain some antigen-binding functions of the whole antigen-binding domain. Examples of antigen binding fragments of an antibody include (1) Fab fragments, monomeric fragments having VL, VH, constant light chain (CL), and CH1 domains; (2) F(ab′)₂ fragments, diatomic fragments having two Fab fragments linked by a disulfide bridge of the hinge region; (3) Fd fragments having two VH and CH1 domains; (4) Fv fragments having single-armed VL and VH domains of antibodies, (5) dAb fragments (Ward et al., “Binding Activities of a Repertoire of Single Immunoglobulin Variable Domains Secreted From Escherichia coli,” Nature 341:544-546 (1989), incorporated by reference into the present disclosure in its entirety), having VH domains; (6) isolated complementary determining region (CDR); (7) single chain Fv (scFv), for example, derived from the scFV library. Although the two domains VL and VH of the Fv fragment are encoded by independent genes, they can be conjugated using a recombination method by a synthetic linker, the synthetic linker enables it to be made into a single protein chain (called as single chain Fv (scFv)) in which the VL and VH regions pair to form a monomeric molecule (see, for example, Huston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-5883 (1988)); and (8) VHH, “VHH” involves variable antigen-binding domains of heavy chain antibodies from the Camelidae (camels, dromedaries, llamas, and alpacas, etc.) (see Nguyen V. K. et al., 2000, The EMBO Journal, 19, 921-930; Muyldermans S., 2001, J Biotechnol., 74, 277-302; and Review Vanlandschoot P. et al., 2011, Antiviral Research 92, 389-407). VHH can also be referred to as nanobody (Nb) and/or single domain antibody. These antibody fragments can be obtained using conventional techniques known to those skilled in the art, and their functionalities can be evaluated in the same way as intact antibodies.

In this disclosure, the terms “variable region” or “variable domain” generally refer to regions where there may be significant differences in sequence of certain segments of the variable domains between antibodies. The “variable region” in a light chain can include the light chain variable region VL; and the “variable region” in a heavy chain can include the heavy chain variable region VH. Variable domains mediate antigen binding and determine the specificity of specific antibodies against their specific antigens. However, variability does not mean the uniform distribution throughout the entire scope of variable domains. It is generally concentrated in three segments called as hypervariable regions (CDR or HVR) in the light chain and heavy chain variable domains. The more highly conserved part of the variable domains is called as the framework region (FR). Each of the variable domains of natural heavy and light chains contains four FR regions, most of which adopt 3-sheet configuration and are connected by three CDRs, forming a circular connection, and in some cases forming a part of a B-sheet structure. The CDRs in each chain are closely held together through the FR region, and promote the formation of antigen-binding sites of antibodies together with the CDRs from another chain (see Kabat et al, Sequences of Immunological Interest, Fifth Edition, National Institute of Health, Bethesda, Md. (1991)). The terms “VH” and “VH domain” can be interchangeably used to refer to the heavy chain variable region of an antibody or antigen-binding molecule thereof.

In this disclosure, the term “CDR” generally refers to the complementary determining region within the antibody variable sequence. There are three CDRs in each variable region of the heavy and light chains, and for each variable region, they are called as CDR1, CDR2, and CDR3, respectively. The precise boundaries of these CDRs have been defined differently based on different systems. The system described by Kabat (Kabat et al., Sequences of Proteins of Immunological Interest (National Institutes of Health, Bethesda, Md. (1987) and (1991)) not only provides a definite residue numbering system applicable to any variable region of the antibody, but also provides precise residue boundaries that define these three CDRs. These CDRs can be referred to as Kabat CDRs. Chothia and co-workers (Chothia & Lesk, J. Mol. Biol. 196:901-917 (1987) and Chothia et al., Nature 342:877-883 (1989)) found that some sub-moieties within Kabat CDR exhibit almost identical peptide skeleton conformations, despite significant differences at the amino acid sequence level. These sub-moieties are designated as L1, L2, and L3, or H1, H2, and H3, where “L” and “H” refer to the light and heavy chain regions, respectively. These regions can be called as Chothia CDRs, which have boundaries that overlap with Kabat CDRs. Padlan (FASEB J. 9:133-139 (1995)) and MacCallum (J Mol Biol 262(5):732-45 (1996)) have already described other boundaries that define CDRs overlapping with Kabat CDRs. Other CDR boundaries may not strictly conform to one of the above systems, but still overlap with Kabat CDR. Although they can be shortened or extended based on predictions or experimental findings below, specific residues or groups of residues, or even the whole CDRs, do not significantly affect the antigen binding. Unless otherwise explicitly stated in the specification, as used in this disclosure, the terms “CDR”, “HCDR1”, “HCDR2”, “HCDR3”, “LCDR1”, “LCDR2”, and “LCDR3” include CDRs as defined in any of the aforementioned methods (Kabat, Chothia, or IMGT).

In this disclosure, the term “percentage (%) sequence identity” generally refers to the number of matches (“hits”) of consistent amino acids when two or more aligned amino acid sequences are compared with the number of amino acid residues making up the total length of the amino acid sequences. In other words, when alignment is used for two or more sequences, the percentage of identical amino acid residues (e.g., 70%, 75%, 80%, 85%, 90%, 95%, 96%, 97%, 98%, or 99% sequence identity) can be determined when comparing and aligning these sequences against the maximum correspondence (as measured using sequence comparison algorithms known in the art), or when manually aligning and visually inspecting. Therefore, sequences that are compared to determine sequence identity can be distinguished by one or more amino acid substitutions, additions, or deletions. The suitable programs for alignment of protein sequences are known to those skilled in the art. The percentage sequence identity of protein sequences can be determined, for example, using programs such as CLUSTALW, Cluster Omega, FASTA, or BLAST, such as the use of NCBI BLAST algorithm (Altschul S F et al. (1997), Nuclear Acids Res. 25:3389-3402).

In the present disclosure, the term “antibody analogue” is generally used in the broadest sense and specifically encompasses molecules that bind specifically to target molecules with monospecificity and are structurally distinct from natural antibodies. For example, in the context of describing an anti-PD-1 antibody or an anti-PD-L1 antibody, the term “antibody analogue” refers to an antibody including a segment having substantial homology to a portion of an amino acid sequence and having at least one of the following properties: (1) specifically binding the PD-1 or the PD-L1 under appropriate binding conditions, and (2) having the capability of inhibiting at least one biological activity of the PD-1 or the PD-L1. Generally, the antibody analogue includes conserved amino acid substitutions (or insertions or deletions) relative to a natural sequence. The analogue is generally at least 20 or 25 amino acids long, at least 50, 60, 70, 80, 90, 100, 150, or 200 amino acids long or longer, and may generally be as long as the full-length heavy or light chain of the antibody. Some examples include the antibody analogs having 1, 2, 3, 4, 5, 6, 7, 8, 9, 10, 11, 12, 13, 14, 15, 16, or 17 substitutions compared to a germline amino acid sequence.

In the present disclosure, the term “pharmaceutical combination” generally refers to a combination including at least two active ingredients/therapeutic agents. In some embodiments, each active ingredient/therapeutic agent can be respectively prepared into an independent preparation (solid, liquid, and gel, etc.). In some embodiments, each active ingredient/therapeutic agent can be contained in different containers, and can also be prepared into the desired preparations concurrently or separately with an appropriate carrier as needed. In some embodiments, each active ingredient/therapeutic agent can be of different sources (e.g., prepared, produced by different manufacturers, or sold by different merchants). In some embodiments, each active ingredient/therapeutic agent can form pharmaceutical compositions in a mixed form. In this disclosure, the term “pharmaceutical composition” generally refers to a preparation being in a form that allows the biological activity of active ingredients to be effective and containing no other components that have unacceptable toxicity to the subject for who the composition is going to be administered. Such composition can be sterile and can contain pharmaceutically acceptable carriers, such as physiological saline. A suitable pharmaceutical composition may include one or more buffer solutions (e.g., acetate, phosphate, or citrate buffer solutions), surfactants (e.g., polysorbate), stabilizers (e.g., human albumin), preservatives (e.g., benzyl alcohol), absorption enhancers for enhancing bioavailability, and/or other conventional solubilizers or dispersants. The pharmaceutical compositions of the disclosure include but are not limited to liquid, frozen, and lyophilized compositions.

In this disclosure, the term “pharmaceutically acceptable carrier” generally refers to one or more non-toxic materials that do not interfere with the biological activity of the active ingredients. Such preparation can conventionally contain a salt, a buffer, a preservative, a compatible carrier, and optionally other therapeutic agents. Such pharmaceutically acceptable preparation can also contain a compatible solid or liquid filler, a diluent, or an encapsulating substance suitable for human administration. Other contemplated carriers, excipients, and/or additives that can be used in the formulations described herein may include, for example, a flavouring agent, an antimicrobial agent, a sweetener, an antioxidant, an antistatic agent, lipid, a protein excipient (e.g., serum albumin, gelatin, casein), a salt-forming counterion (e.g., sodium), and the like. These and other known pharmaceutical carriers, excipients, and/or additives suitable for use in the formulations described herein are known in the art.

In this disclosure, the term “neoplastic cells” generally refers to cells that undergo new and abnormal proliferation, especially those in which proliferation is uncontrollable and progresses, leading to neoplastic diseases. Neoplastic cells can be malignant, i.e., invasive and metastatic, or benign.

In this disclosure, the term “neoplasm” generally refers to an abnormal lump of tissues, where the growth of the lump exceeds that of normal tissues and is not coordinated with the growth of normal tissues. The “neoplasm” can be defined to be “benign” or “malignant”, depending on the following characteristics: degree of cell differentiation, including morphology and function, rate of growth, local invasion and metastasis. “Benign neoplasm” is generally well-differentiated, has slower growth characteristics than malignant neoplasm, and maintains localization to the origin site. In addition, benign tumors do not have the ability to infiltrate, invade, or metastasize to the distal sites. “Malignant neoplasm” is generally poorly differentiated (anaplastic) and has characteristic rapid growth, accompanied by progressive infiltration, invasion, and destruction of surrounding tissues. In addition, malignant neoplasm has the ability to metastasize to the distal sites.

In this disclosure, the term “tumor” or “cancer” generally refers to any medical disorders characterized by the growth, proliferation, or metastasis of neoplastic or malignant cells, and the tumor can be a solid or non-solid tumor.

In this disclosure, the term “wart” generally refers to a type of superficial benign neoplasm of the skin caused by human papilloma virus (HPV), mainly characterized by cell proliferative responses. HPV belongs to the Papovaviridae A genus in the DNA virus family. HPV is divided into more than 80 subtypes and is associated with different types of warts. The term “wart” generally refers to all types of warts, including but not limiting to verruca plantaris, verruca vulgaris, and genital warts, unless otherwise specified.

In this disclosure, the term “administer” and similar terms are generally not limited to bodily administration, and suitable methods include in vitro, in vitro followed by in vivo, or in vivo methods. For example, any administration method known to those skilled in the art for bringing cells, organs, or tissues into contact with the composition can be used. For example, the compound can be introduced into the body of a subject in need of treatment through any introduction or delivery routes. In some embodiments, the composition of the disclosure can be administered orally, locally, intranasally, intramuscularly, subcutaneously, intradermally, intrathecally, intraperitoneally, percutaneously, or intratumorally.

In this disclosure, the term “effective amount” or “effective dose” generally refers to an amount sufficient to achieve or at least partially achieve the desired effect. The “therapeutically effective amount” or “therapeutically effective dose” of a drug or therapeutic agent is generally any amount of a drug that promotes regression of a disease when used alone or in combination with another therapeutic agent (as evidenced by a decrease in the severity of disease symptoms, an increase in the frequency and duration of asymptomatic periods of a disease, or the prevention of damage or disability caused by suffering from a disease).

In this disclosure, the term “treatment” generally refers to the slowing down or improvement of the progression, severity, and/or duration of proliferative disorders, or the improvement of one or more symptoms of proliferative disorders (e.g., one or more identifiable symptoms) due to the administration of one or more therapies (e.g., one or more therapeutic agents such as the pharmaceutical composition of this disclosure). In this disclosure, the term “treatment” may also refer to the improvement of at least one measurable physical parameter of proliferative disorders, such as tumor growth, not necessarily to be distinguishable by the patient. The term “treatment” in this disclosure can also refer to the inhibition of the progression of proliferative disorders through, for example, stabilizing the identifiable symptoms by physical means (for example, stabilizing physical parameters), or by physiological means, or both. In some cases, the term “treatment” can refer to reduction or stabilization of tumor size or cancer cell counts.

In this disclosure, the term “synergy” generally refers to the efficacy of the combination of two or more active drugs being greater than the sum of efficacies obtained by using the individual drugs alone. Therefore, when the combination of two or more pharmaceutical ingredients leads to a “synergistic inhibition” of an activity or process, such as tumor growth, it means that the inhibitory effect on the activity or process is greater than the sum of the inhibitory effects of the active drugs alone.

In this disclosure, the term “subject” generally refers to human or non-human animals, including but not limiting to cats, dogs, horses, pigs, cows, sheep, rabbits, mice, rats, or monkeys, etc.

In this disclosure, the term “about” generally refers to a variation within a range of (0.5% to 10%) above or below the specified value, such as a variation within a range of about 0.5%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% above or below the specified value.

In this disclosure, the term “comprising” and its variant forms including “containing”, “including” and other forms, generally refer to the inclusion of other components, elements, numerical values, and steps, etc.

### DETAILED DESCRIPTION OF INVENTION

This disclosure provides an agonist that induces the expression of INF and cytokines as well as activates innate immune system responses in combination with an immune checkpoint inhibitor for use as a pharmaceutical combination, including a TLR (for example, a TLR7/TLR8) agonist and an immune checkpoint (for example, PD-1 or PD-L1) inhibitor, etc.

In one aspect, the disclosure provides a pharmaceutical combination, which can include a programmed cell death protein 1 (PD-1) inhibitor and/or a programmed death ligand 1 (PD-L1) inhibitor, and a TLR agonist.

In an aspect, the present disclosure provides a pharmaceutical combination, which may include a PD-1 inhibitor and/or a PD-L1 inhibitor, and a TLR agonist.

In some implementations, a TLR agonist may include a TLR1, a TLR2, a TLR3, a TLR4, a TLR5, a TLR6, a TLR7, a TLR8, a TLR9, and/or a TLR10.

In some implementations, the TLR agonist is selected from one or more of a TLR7 agonist, a TLR8 agonist, or a TLR9 agonist.

In some implementations, the TLR agonist includes a TLR7 and TLR8 dual agonist (TLR7/TLR8 agonist).

In some implementations, the TLR agonist includes the imidazole quinoline derivative.

In some implementations, the TLR agonist is selected from one or more of Imiquimod, Gardiquimod, Resiquimod, 1V209, Selgantolimod (GS-9688), Vesatolimod (GS-9620), Sumanirole, PF-4878691, or pharmaceutically acceptable derivatives thereof.

In some implementations, the TLR agonist includes the Imiquimod, Resiquimod, or pharmaceutically acceptable salts thereof.

In some implementations, the TLR agonist is selected from one or more of LHC-165, NKTR-262, DN1508052-01, SHR2150, CL307, CL264, Loxoribine, Isatoribine, DSR-6434, GSK2245035, SM-276001, SM-324405, SM-324406, AZ12441970, AZ12443988, or pharmaceutically acceptable derivatives thereof.

In some embodiments, where the PD-1 inhibitor has one or more of the following characteristics:
a. inhibition or reduction of PD-1 expression, such as transcription or translation of PD-1;
b. inhibition or reduction of PD-1 activity, such as inhibition or reduction of PD-1 binding to its homologous ligands, such as PD-L1 or PD-L2; and
c. binding PD-1 or one or more of its ligands, such as PD-L1 or PD-L2.

In some embodiments, where the PD-1 inhibitor includes an anti PD-1 antibody or antigen-binding fragments thereof.

For example, the pharmaceutical combination can include: 1) an anti PD-1 antibody or antigen-binding fragments thereof; and 2) TLR7 and/or TLR8 agonist.

For another example, the pharmaceutical combination can include: 1) the anti-PD-1 antibody or the antigen binding fragment thereof; and 2) an imidazole quinoline derivative.

In some embodiments, where the anti PD-1 antibody is selected from the group consisting of Pembrolizumab, Nivolumab, Pidilizumab, Tislelizumab, SHR-1210 (Incyte/Jiangsu Hengrui Pharmaceuticals Co., Ltd.), MEDI0680 (also known as AMP-514; Amplimmune Inc./Medimmune), BGB-A317 (BeiGene Ltd.), TSR-042 (also known as ANB011; AnaptysBio/Tesaro, nc.), REGN2810 (Regeneron Pharmaceuticals, Inc./Sanofi-Aventis), PF-06801591 (Pfizer), RB0004, analogues thereof, or combinations thereof.

For example, the pharmaceutical combination can include: 1) an anti PD-1 antibody, which can be selected from the group consisting of Pembrolizumab, Nivolumab, Pidilizumab, Tislelizumab, SHR-1210 (Incyte/Jiangsu Hengrui Pharmaceuticals Co., Ltd.), MEDI0680 (also known as AMP-514; Amplimmune Inc./Medimmune), BGB-A317 (BeiGene Ltd.), TSR-042 (also known as ANB011; AnaptysBio/Tesaro, nc.), REGN2810 (Regeneron Pharmaceuticals, Inc./Sanofi-Aventis), PF-06801591 (Pfizer), RB0004, analogues thereof, or combinations thereof; and 2) the imidazole quinoline derivative, where the imidazole quinoline derivative may be selected from Imiquimod, Gardiquimod, Resiquimod, derivatives thereof, andor a combination thereof.

In some embodiments, the anti PD-1 antibody is RB0004. RB0004 and other humanized anti PD-1 monoclonal antibodies are disclosed in CN201610345750.1 and WO2017201766A1. For example, the pharmaceutical combination includes: 1) the anti-PD-1 antibody, where the anti-PD-1 antibody is the RB0004 or the analogues thereof, and a combination thereof; and 2) the imidazole quinoline derivative, where the imidazole quinoline derivative may be the Imiquimod or the derivatives thereof, and a combination thereof.

In another embodiments, the antibody PD-1 is Pembrolizumab (trade name Keytruda, previously Lambrolzumab, also known as Merck 3745, MK-3475, or SCH-900475), a humanized IgG4 monoclonal antibody that binds to PD-1. Pembrolizumab is disclosed in, for example Hamid et al. (2013) New England Journal of Medicine 369(2):134-44, WO2009/114335 and U.S. Pat. No. 8,354,509. For example, the pharmaceutical combination includes: 1) the anti-PD-1 antibody, where the anti-PD-1 antibody may be the Pembrolizumab or a biological enhancer thereof, a biological equivalent thereof, and a combination thereof; and 2) the imidazole quinoline derivative, where the imidazole quinoline derivative may be the Imiquimod or the derivatives thereof, and a combination thereof.

In another embodiments, the anti PD-1 antibody is Nivolumab (CAS Registry No.: 946414-94-4, alternative names include MDX-1106, MDX-1106-04, ONO-4538, or BMS-936558). Nivolumab is a complete human IgG4 monoclonal antibody that specifically blocks PD-1. Nivolumab (Clone 5C4) and other human monoclonal antibodies specifically binding to PD-1 have been disclosed in U.S. Pat. No. 8,008,449 and WO2006/121168. For example, the pharmaceutical combination includes: 1) the anti-PD-1 antibody, where the anti-PD-1 antibody may be the Nivolumab or the analogues thereof, and a combination thereof; and 2) the imidazole quinoline derivative, where the imidazole quinoline derivative may be the Imiquimod or the derivatives thereof, and a combination thereof.

In another embodiments, the anti PD-1 antibody is Pidilizumab. Pidilizumab (CT-011; Cure Tech) is a humanized IgGI monoclonal antibody that binds to PD-1. Pidilizumab and other humanized anti PD-1 monoclonal antibodies are disclosed in WO2009/101611. Other anti Rpd-1 antibodies have been disclosed in U.S. Pat. No. 8,609,089, US2010028330, and/or US20120114649. Other anti PD-1 antibodies include AMP514 (Amplimmune). For example, the pharmaceutical combination includes: 1) the anti-PD-1 antibody, where the anti-PD-1 antibody may be the Pidilizumab or the analogues thereof, and a combination thereof; and 2) the imidazole quinoline derivative, where the imidazole quinoline derivative may be the Imiquimod or the derivatives thereof, and a combination thereof.

In some embodiments, where the anti PD-1 antibody includes at least one CDR in the antibody heavy chain variable region (VH), and the VH includes an amino acid sequence shown in SEQ ID NO: 8.

In some embodiments, where the anti PD-1 antibody includes VH including HCDR3, the HCDR3 includes an amino acid sequence shown in SEQ ID NO: 3 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 3.

In some embodiments, where the VH further includes HCDR2, wherein the HCDR2 includes an amino acid sequence shown in SEQ ID NO: 2 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 2.

In some embodiments, where the VH further includes HCDR1, wherein the HCDR1 includes an amino acid sequence shown in SEQ ID NO: 1 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 1.

In some embodiments, where the VH includes HCDR1, HCDR2, and HCDR3, wherein the HCDR3 includes an amino acid sequence shown in SEQ ID NO: 3 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 3, the HCDR2 includes an amino acid sequence shown in SEQ ID NO: 2 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 2, and the HCDR1 includes an amino acid sequence shown in SEQ ID NO: 1 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 1.

In some embodiments, where the VH includes a framework region HFR1, wherein the C-terminal of HFR1 is directly or indirectly connected to the N-terminal of HCDR1, and the HFR1 includes an amino acid sequence shown in SEQ ID NO: 4 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 4.

In some embodiments, where the VH includes a framework region HFR2, wherein the N-terminal of HFR2 is directly or indirectly connected to the C-terminal of HCDR1, and the C-terminal of HFR2 is directly or indirectly connected to the N-terminal of HCDR2; and the HFR2 includes an amino acid sequence shown in SEQ ID NO: 5 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 5.

In some embodiments, where the VH includes a framework region HFR3, wherein the N-terminal of HFR3 is directly or indirectly connected to the C-terminal of HCDR2, and the C-terminal of HFR3 is directly or indirectly connected to the N-terminal of HCDR3; and the HFR3 includes an amino acid sequence shown in SEQ ID NO: 6 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 6.

In some embodiments, where the VH includes a framework region HFR4, wherein the N-terminal of HFR4 is directly or indirectly connected to the C-terminal of HCDR3, and the HFR4 includes an amino acid sequence shown in SEQ ID NO: 7 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 7.

In some embodiments, where the VH includes framework regions HFR1, HFR2, HFR3, and HFR4, the C-terminal of HFR1 is directly or indirectly connected to the N-terminal of HCDR1, the N-terminal of HFR2 is directly or indirectly connected to the C-terminal of HCDR1, and the C-terminal of HFR2 is directly or indirectly connected to the N-terminal of HCDR2, the N-terminal of HFR3 is directly or indirectly connected to the C-terminal of HCDR2, and the C-terminal of HFR3 is directly or indirectly connected to the N-terminal of HCDR3, the N-terminal of HFR4 is directly or indirectly connected to the C-terminal of HCDR3; among them, the HFR1 includes an amino acid sequence shown in SEQ ID NO: 4 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 4, the HFR2 includes an amino acid sequence shown in SEQ ID NO: 5 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 5, the HFR3 includes an amino acid sequence shown in SEQ ID NO: 6 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 6, and the HFR4 includes an amino acid sequence shown in SEQ ID NO: 7 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 7.

In some embodiments, where the anti PD-1 antibody includes VH, the VH includes an amino acid sequence shown in SEQ ID NO: 8 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 8.

In some embodiments, where the anti PD-1 antibody includes an antibody heavy chain (HC), the HC includes an amino acid sequence shown in SEQ ID NO: 9 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 9.

In some embodiments, where the anti PD-1 antibody includes at least one CDR in the antibody light chain variable region (VL), and the VL includes an amino acid sequence shown in SEQ ID NO: 17.

In some embodiments, where the anti PD-1 antibody includes at least one CDR in VH, wherein the VH includes an amino acid sequence shown in SEQ ID NO: 8, and the anti PD-1 antibody includes at least one CDR in VL, and the VL includes an amino acid sequence shown in SEQ ID NO: 17.

In some embodiments, where the anti PD-1 antibody includes VL including LCDR1, the LCDR1 includes an amino acid sequence shown in SEQ ID NO: 10 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 10.

In some embodiments, where the VL further includes LCDR2, wherein the LCDR2 includes an amino acid sequence shown in SEQ ID NO: 11 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 11.

In some embodiments, where the VL further includes LCDR3, wherein the LCDR3 includes an amino acid sequence shown in SEQ ID NO: 12 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 12.

In some embodiments, where the VL includes LCDR1, LCDR2 and LCDR3, wherein the LCDR1 includes an amino acid sequence shown in SEQ ID NO: 10 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 10, the LCDR2 includes an amino acid sequence shown in SEQ ID NO: 11 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 11, and the LCDR3 includes an amino acid sequence shown in SEQ ID NO: 12 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 12.

In some embodiments, where the anti PD-1 antibody includes VH and an antibody VL, the VH includes HCDR1, HCDR2, and HCDR3, wherein the HCDR3 includes an amino acid sequence shown in SEQ ID NO: 3 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 3, the HCDR2 includes an amino acid sequence shown in SEQ ID NO: 2 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 2, and the HCDR1 includes an amino acid sequence shown in SEQ ID NO: 1 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 1; and the VL includes LCDR1, LCDR2, and LCDR3, wherein the LCDR1 includes an amino acid sequence shown in SEQ ID NO: 10 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 10, the LCDR2 includes an amino acid sequence shown in SEQ ID NO: 11 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 11, and the LCDR3 includes an amino acid sequence shown in SEQ ID NO: 12 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 12.

For example, the anti PD-1 antibody can include VH and antibody VL, the VH can include HCDR1, HCDR2, and HCDR3, wherein the HCDR3 can include an amino acid sequence shown in SEQ ID NO: 3, the HCDR2 can include an amino acid sequence shown in SEQ ID NO: 2, and the HCDR1 can include an amino acid sequence shown in SEQ ID NO: 1; and the VL can include LCDR1, LCDR2, and LCDR3, wherein the LCDR1 can include an amino acid sequence shown in SEQ ID NO: 10, the LCDR2 can include an amino acid sequence shown in SEQ ID NO: 11, and the LCDR3 can include an amino acid sequence shown in SEQ ID NO: 12.

For example, the pharmaceutical combination can include: 1) anti PD-1 antibody, the anti PD-1 antibody can include VH and antibody VL, the VH can include HCDR1, HCDR2, and HCDR3, wherein the HCDR3 can include an amino acid sequence shown in SEQ ID NO: 3, the HCDR2 can include an amino acid sequence shown in SEQ ID NO: 2, and the HCDR1 can include an amino acid sequence shown in SEQ ID NO: 1; and the VL can include LCDR1, LCDR2, and LCDR3, wherein the LCDR1 can include an amino acid sequence shown in SEQ ID NO: 10, the LCDR2 can include an amino acid sequence shown in SEQ ID NO: 11, and the LCDR3 can include an amino acid sequence shown in SEQ ID NO: 12; 2) the imidazole quinoline derivative, where the imidazole quinoline derivative may be the Imiquimod or the derivatives thereof, and a combination thereof.

In some embodiments, where the VL includes a framework region LFR1, the C-terminal of LFR1 is directly or indirectly connected to the N-terminal of LCDR1, and the LFR1 includes an amino acid sequence shown in SEQ ID NO: 13 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 13.

In some embodiments, where the VL includes a framework region LFR2, wherein the N-terminal of LFR2 is directly or indirectly connected to the C-terminal of LCDR1, and the C-terminal of LFR2 is directly or indirectly connected to the N-terminal of LCDR2; and the LFR2 includes an amino acid sequence shown in SEQ ID NO: 14 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 14.

In some embodiments, where the VL includes a framework region LFR3, wherein the N-terminal of LFR3 is directly or indirectly connected to the C-terminal of LCDR2, and the C-terminal of LFR3 is directly or indirectly connected to the N-terminal of LCDR3; and the LFR3 includes an amino acid sequence shown in SEQ ID NO: 15 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 15.

In some embodiments, where the VL includes a framework region LFR4, wherein the N-terminal of LFR4 is directly or indirectly connected to the C-terminal of LCDR3, and the LFR4 includes an amino acid sequence shown in SEQ ID NO: 16 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 16.

In some embodiments, where the VL includes framework regions LFR1, LFR2, LFR3 and LFR4, the C-terminal of LFR1 is directly or indirectly connected to the N-terminal of LCDR1, the N-terminal of LFR2 is directly or indirectly connected to the C-terminal of LCDR1, and the C-terminal of LFR2 is directly or indirectly connected to the N-terminal of LCDR2, the N-terminal of LFR3 is directly or indirectly connected to the C-terminal of LCDR2, and the C-terminal of LFR3 is directly or indirectly connected to the N-terminal of LCDR3, the N-terminal of LFR4 is directly or indirectly connected to the C-terminal of LCDR3; among them, the LFR1 includes an amino acid sequence shown in SEQ ID NO: 13 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 13, the LFR2 includes an amino acid sequence shown in SEQ ID NO: 14 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 14, the LFR3 includes an amino acid sequence shown in SEQ ID NO: 15 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 15, and the LFR4 includes an amino acid sequence shown in SEQ ID NO: 16 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 16.

In some embodiments, where the anti PD-1 antibody includes VL, the VL includes an amino acid sequence shown in SEQ ID NO: 17 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 17.

In some embodiments, where the anti PD-1 antibody includes VH and VL, the VH includes an amino acid sequence shown in SEQ ID NO: 8 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 8, and the VL includes an amino acid sequence shown in SEQ ID NO: 17 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 17.

For example, where the anti PD-1 antibody can include VH and VL, the VH can include an amino acid sequence shown in SEQ ID NO: 8, and the VL can include an amino acid sequence shown in SEQ ID NO: 17.

For example, the pharmaceutical combination can include: 1) anti PD-1 antibody, wherein the anti PD-1 antibody can include VH and VL, the VH can include an amino acid sequence shown in SEQ ID NO: 8, and the VL can include an amino acid sequence shown in SEQ ID NO: 17; 2) the imidazole quinoline derivative, where the imidazole quinoline derivative may be the Imiquimod or the derivatives thereof, and a combination thereof.

In some embodiments, where the anti PD-1 antibody includes an antibody light chain (LC), the LC includes an amino acid sequence shown in SEQ ID NO: 18 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 18.

In some embodiments, where the anti PD-1 antibody includes HC and LC, the HC includes an amino acid sequence shown in SEQ ID NO: 9 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 9, and the LC includes an amino acid sequence shown in SEQ ID NO: 18 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 18.

For example, where the anti PD-1 antibody can include HC and LC, the HC can include an amino acid sequence shown in SEQ ID NO: 9, and the LC can include an amino acid sequence shown in SEQ ID NO: 18.

For example, the pharmaceutical combination can include: 1) anti PD-1 antibody, wherein the anti PD-1 antibody can include HC and LC, the HC can include an amino acid sequence shown in SEQ ID NO: 9, and the LC can include an amino acid sequence shown in SEQ ID NO: 18; 2) the imidazole quinoline derivative, where the imidazole quinoline derivative may be the Imiquimod or the derivatives thereof, and a combination thereof.

In some embodiments, where the PD-L1 inhibitor has one or more of the following characteristics:
a. inhibition or reduction of PD-L1 expression, such as transcription or translation of PD-L1;
b. inhibition or reduction of PD-L1 activity, such as inhibition or reduction of PD-L1 binding to its associated receptors such as PD-1; and
c. binding of PD-L1 or its receptors such as PD-1.

In some embodiments, where the PD-L1 inhibitor includes an anti PD-L1 antibody or antigen-binding fragments thereof.

For example, the pharmaceutical combination can include: 1) an anti PD-L1 antibody or antigen-binding fragments thereof; and 2) TLR7 and/or TLR8 agonist.

For another example, the pharmaceutical combination may include: 1) the anti-PD-L1 antibody or the antigen binding fragment thereof; and 2) an imidazole quinoline derivative.

In some embodiments, where the anti PD-L1 antibody is selected from the group consisting of Durvalumab (MEDI4736, disclosed in US2013/0034559A1), Atezolizumab (MPDL3280A, disclosed in U.S. Pat. No. 8,217,149), Avelumab (MSB0010718C, disclosed in US2014/0341917A1), MDX-1105, YW243.55.S70, MDPL3280A, AMP-224 (Amplimume, GlaxoSmithKline), LY3300054 (Eli Lilly and Co.), RB0005, analogues, or combinations thereof.

For example, the pharmaceutical combination can include: 1) an anti PD-L1 antibody, wherein the anti PD-L1 antibody is selected from the group consisting of Durvalumab (MEDI4736, disclosed in US2013/0034559A1), Atezolizumab (MPDL3280A, disclosed in U.S. Pat. No. 8,217,149), Avelumab (MSB0010718C, disclosed in US2014/0341917A1), MDX-1105, YW243.55.S70, MDPL3280A, AMP-224, LY3300054, RB0005, analogues thereof, and a combination thereof; and 2) the imidazole quinoline derivative, where the imidazole quinoline derivative may be selected from Imiquimod, Gardiquimod, Resiquimod, derivatives thereof, andor a combination thereof.

In some embodiments, the anti PD-L1 antibody is RB0005. RB0005 and other humanized anti PD-L1 monoclonal antibodies have been disclosed in CN201610340678.3 and WO2017197667A1. For example, the pharmaceutical combination includes: 1) the anti-PD-L1 antibody, where the anti-PD-L1 antibody is the RB0005 or the analogues thereof, and a combination thereof; and 2) the imidazole quinoline derivative, where the imidazole quinoline derivative may be the Imiquimod or the derivatives thereof, and a combination thereof.

In other embodiments, the PD-L1 inhibitor is MDX-1105. MDX-1105, also known as BMS-936559, is an anti PD-L1 antibody described in WO 2007/005874. For example, the pharmaceutical combination includes: 1) the anti-PD-L1 antibody, where the anti-PD-L1 antibody is the MDX-1105 or the analogues thereof, and a combination thereof; and 2) the imidazole quinoline derivative, where the imidazole quinoline derivative may be the Imiquimod or the derivatives thereof, and a combination thereof.

In other embodiments, the PD-L1 inhibitor is YW243.55.S70. The YW243.55.S70 antibody is an anti PD-L1 antibody described in WO 2010/077634.

In other embodiments, the PD-L1 inhibitor is MDPL3280A (Genentech/Roche). MDPL3280A is a human Fc optimized IgG1 monoclonal antibody that binds to PD-L1. Other human monoclonal antibodies of MDPL3280A and PD-L1 have been disclosed in U.S. Pat. No. 7,943,743 and US patent publication number 20120039906.

In some embodiments, where the anti PD-L1 antibody includes at least one CDR in VH, the VH includes an amino acid sequence shown in SEQ ID NO: 25.

In some embodiments, where the anti PD-L1 antibody includes VH including HCDR3, the HCDR3 includes an amino acid sequence shown in SEQ ID NO: 21 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 21.

In some embodiments, where the VH further includes HCDR2, wherein the HCDR2 includes an amino acid sequence shown in SEQ ID NO: 20 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 20.

In some embodiments, where the VH further includes HCDR1, wherein the HCDR1 includes an amino acid sequence shown in SEQ ID NO: 19 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 19.

In some embodiments, where the VH includes HCDR1, HCDR2 and HCDR3, wherein the HCDR3 includes an amino acid sequence shown in SEQ ID NO: 21 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 21, the HCDR2 includes an amino acid sequence shown in SEQ ID NO: 20 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 20, and the HCDR1 includes an amino acid sequence shown in SEQ ID NO: 19 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 19.

For example, where the VH can include HCDR1, HCDR2, and HCDR3, wherein the HCDR3 includes an amino acid sequence shown in SEQ ID NO: 21, the HCDR2 includes an amino acid sequence shown in SEQ ID NO: 20, and the HCDR1 includes an amino acid sequence shown in SEQ ID NO: 19.

In some embodiments, where the VH includes a framework region HFR1, the C-terminal of HFR1 is directly or indirectly connected to the N-terminal of HCDR1, and the HFR1 includes an amino acid sequence shown in SEQ ID NO: 22 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 22.

In some embodiments, where the VH includes a framework region HFR2, the N-terminal of HFR2 is directly or indirectly connected to the C-terminal of HCDR1, and the C-terminal of HFR2 is directly or indirectly connected to the N-terminal of HCDR2; and the HFR2 includes an amino acid sequence shown in SEQ ID NO: 23 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 23.

In some embodiments, where the VH includes a framework region HFR3, the N-terminal of HFR3 is directly or indirectly connected to the C-terminal of HCDR2, and the C-terminal of HFR3 is directly or indirectly connected to the N-terminal of HCDR3; and the HFR3 includes an amino acid sequence shown in SEQ ID NO: 24 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 24.

In some embodiments, where the VH includes a framework region HFR4, the N-terminal of HFR4 is directly or indirectly connected to the C-terminal of HCDR3, and the HFR4 includes an amino acid sequence shown in SEQ ID NO: 7 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 7.

In some embodiments, where the VH includes framework regions HFR1, HFR2, HFR3, and HFR4, the C-terminal of HFR1 is directly or indirectly connected to the N-terminal of HCDR1, the N-terminal of HFR2 is directly or indirectly connected to the C-terminal of HCDR1, and the C-terminal of HFR2 is directly or indirectly connected to the N-terminal of HCDR2, the N-terminal of HFR3 is directly or indirectly connected to the C-terminal of HCDR2, and the C-terminal of HFR3 is directly or indirectly connected to the N-terminal of HCDR3, the N-terminal of HFR4 is directly or indirectly connected to the C-terminal of HCDR3; among them, the HFR1 includes an amino acid sequence shown in SEQ ID NO: 22 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 22, the HFR2 includes an amino acid sequence shown in SEQ ID NO: 23 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 23, the HFR3 includes an amino acid sequence shown in SEQ ID NO: 24 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 24, and the HFR4 includes an amino acid sequence shown in SEQ ID NO: 7 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 7.

In some embodiments, where the anti PD-L1 antibody includes VH, and the VH includes an amino acid sequence shown in SEQ ID NO: 25 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 25.

In some embodiments, where the anti PD-L1 antibody includes HC, and the HC includes an amino acid sequence shown in SEQ ID NO: 26 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 26.

In some embodiments, where the anti PD-L1 antibody includes at least one CDR in VL, and the VL includes an amino acid sequence shown in SEQ ID NO: 37.

In some embodiments, where the anti PD-L1 antibody includes at least one CDR in VH, the VH includes an amino acid sequence shown in SEQ ID NO: 25, and the anti PD-L1 antibody includes at least one CDR in VL, and the VL includes an amino acid sequence shown in SEQ ID NO: 37.

In some embodiments, where the anti PD-L1 antibody includes at least one CDR in VH, the VH includes an amino acid sequence shown in SEQ ID NO: 25, and the anti PD-L1 antibody includes at least one CDR in VL, and the VL includes an amino acid sequence shown in SEQ ID NO:38, SEQ ID NO: 39 or SEQ ID NO:40.

In some embodiments, where the anti PD-L1 antibody includes VL including LCDR1, the LCDR1 includes an amino acid sequence shown in SEQ ID NO: 27 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 27.

In some embodiments, where the anti PD-L1 antibody includes VL including LCDR1, the LCDR1 includes an amino acid sequence shown in SEQ ID NO:28, SEQ ID NO:29 or SEQ ID NO: 30 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:28, SEQ ID NO:29 or SEQ ID NO:30.

In some embodiments, where the VL further includes LCDR2, wherein the LCDR2 includes an amino acid sequence shown in SEQ ID NO: 31 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 31.

In some embodiments, where the VL further includes LCDR3, wherein the LCDR3 includes an amino acid sequence shown in SEQ ID NO: 32 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 32.

In some embodiments, where the VL includes LCDR1, LCDR2 and LCDR3, wherein the LCDR1 includes an amino acid sequence shown in SEQ ID NO: 27 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:27, the LCDR2 includes an amino acid sequence shown in SEQ ID NO: 31 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 31, and the LCDR3 includes an amino acid sequence shown in SEQ ID NO: 32 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 32.

In some embodiments, where the VL includes LCDR1, LCDR2 and LCDR3, wherein the LCDR1 includes an amino acid sequence shown in SEQ ID NO: 28 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:28, the LCDR2 includes an amino acid sequence shown in SEQ ID NO: 31 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 31, and the LCDR3 includes an amino acid sequence shown in SEQ ID NO: 32 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 32;

In some embodiments, where the VL includes LCDR1, LCDR2 and LCDR3, wherein the LCDR1 includes an amino acid sequence shown in SEQ ID NO: 29 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:29, the LCDR2 includes an amino acid sequence shown in SEQ ID NO: 31 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 31, and the LCDR3 includes an amino acid sequence shown in SEQ ID NO: 32 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 32; or

In some embodiments, where the VL includes LCDR1, LCDR2 and LCDR3, wherein the LCDR1 includes an amino acid sequence shown in SEQ ID NO: 30 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 30, the LCDR2 includes an amino acid sequence shown in SEQ ID NO: 31 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 31, and the LCDR3 includes an amino acid sequence shown in SEQ ID NO: 32 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 32.

In some embodiments, where the anti PD-L1 antibody includes VH and an antibody VL, the VH includes HCDR1, HCDR2, and HCDR3, wherein the HCDR3 includes an amino acid sequence shown in SEQ ID NO: 21 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 21, the HCDR2 includes an amino acid sequence shown in SEQ ID NO: 20 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 20, and the HCDR1 includes an amino acid sequence shown in SEQ ID NO: 19 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 19; and the VL includes LCDR1, LCDR2, and LCDR3, wherein the LCDR1 includes an amino acid sequence shown in SEQ ID NO: 27 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 27, the LCDR2 includes an amino acid sequence shown in SEQ ID NO: 31 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 31, and the LCDR3 includes an amino acid sequence shown in SEQ ID NO: 32 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 32.

For example, where the anti PD-L1 antibody can include VH and an antibody VL, the VH can include HCDR1, HCDR2, and HCDR3, wherein the HCDR3 can include an amino acid sequence shown in SEQ ID NO: 21, the HCDR2 can include an amino acid sequence shown in SEQ ID NO: 20, and the HCDR1 can include an amino acid sequence shown in SEQ ID NO: 19; and the VL can include LCDR1, LCDR2, and LCDR3, wherein the LCDR1 can include an amino acid sequence shown in SEQ ID NO: 27, the LCDR2 can include an amino acid sequence shown in SEQ ID NO: 31, and the LCDR3 can include an amino acid sequence shown in SEQ ID NO: 32.

For example, the pharmaceutical combination can include: 1) anti PD-L1 antibody, wherein the anti PD-L1 antibody can include VH and antibody VL, the VH can include HCDR1, HCDR2, and HCDR3, wherein the HCDR3 can include an amino acid sequence shown in SEQ ID NO: 21, the HCDR2 can include an amino acid sequence shown in SEQ ID NO: 20, and the HCDR1 can include an amino acid sequence shown in SEQ ID NO: 19; and the VL can include LCDR1, LCDR2, and LCDR3, wherein the LCDR1 can include an amino acid sequence shown in SEQ ID NO: 27, the LCDR2 can include an amino acid sequence shown in SEQ ID NO: 31, and the LCDR3 can include an amino acid sequence shown in SEQ ID NO: 32; 2) the imidazole quinoline derivative, where the imidazole quinoline derivative may be the Imiquimod or the derivatives thereof, and a combination thereof.

In some embodiments, where the anti PD-L1 antibody includes VH and an antibody VL, the VH includes HCDR1, HCDR2, and HCDR3, wherein the HCDR3 includes an amino acid sequence shown in SEQ ID NO: 21 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 21, the HCDR2 includes an amino acid sequence shown in SEQ ID NO: 20 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 20, and the HCDR1 includes an amino acid sequence shown in SEQ ID NO: 19 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 19; and the VL includes LCDR1, LCDR2, and LCDR3, wherein the LCDR1 includes an amino acid sequence shown in SEQ ID NO:28, SEQ ID NO: 29 or SEQ ID NO:30 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:28, SEQ ID NO:29 or SEQ ID NO:30, the LCDR2 includes an amino acid sequence shown in SEQ ID NO: 31 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 31, and the LCDR3 includes an amino acid sequence shown in SEQ ID NO: 32 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 32.

For example, where the anti PD-L1 antibody can include VH and an antibody VL, the VH can include HCDR1, HCDR2, and HCDR3, wherein the HCDR3 can include an amino acid sequence shown in SEQ ID NO: 21, the HCDR2 can include an amino acid sequence shown in SEQ ID NO: 20, and the HCDR1 can include an amino acid sequence shown in SEQ ID NO: 19; and the VL include LCDR1, LCDR2, and LCDR3, wherein the LCDR1 can include an amino acid sequence shown in SEQ ID NO:28, SEQ ID NO:29 or SEQ ID NO:30, the LCDR2 can include an amino acid sequence shown in SEQ ID NO: 31, and the LCDR3 can include an amino acid sequence shown in SEQ ID NO: 32.

For example, the pharmaceutical combination can include: 1) anti PD-L1 antibody, wherein the anti PD-L1 antibody can include VH and antibody VL, the VH can include HCDR1, HCDR2, and HCDR3, wherein the HCDR3 can include an amino acid sequence shown in SEQ ID NO: 21, the HCDR2 can include an amino acid sequence shown in SEQ ID NO: 20, and the HCDR1 can include an amino acid sequence shown in SEQ ID NO: 19; and the VL can include LCDR1, LCDR2, and LCDR3, wherein the LCDR1 can include an amino acid sequence shown in SEQ ID NO:28, SEQ ID NO: 29 or SEQ ID NO:30, the LCDR2 can include an amino acid sequence shown in SEQ ID NO: 31, and the LCDR3 can include an amino acid sequence shown in SEQ ID NO: 32; 2) the imidazole quinoline derivative, where the imidazole quinoline derivative may be the Imiquimod or the derivatives thereof, and a combination thereof.

In some embodiments, where the VL includes a framework region LFR1, the C-terminal of LFR1 is directly or indirectly connected to the N-terminal of LCDR1, and the LFR1 includes an amino acid sequence shown in SEQ ID NO: 33 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 33.

In some embodiments, where the VL includes a framework region LFR2, wherein the N-terminal of LFR2 is directly or indirectly connected to the C-terminal of LCDR1, and the C-terminal of LFR2 is directly or indirectly connected to the N-terminal of LCDR2; and the LFR2 includes an amino acid sequence shown in SEQ ID NO: 34 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 34.

In some embodiments, where the VL includes a framework region LFR3, wherein the N-terminal of LFR3 is directly or indirectly connected to the C-terminal of LCDR2, and the C-terminal of LFR3 is directly or indirectly connected to the N-terminal of LCDR3; and the LFR3 includes an amino acid sequence shown in SEQ ID NO: 35 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 35.

In some embodiments, where the VL includes a framework region LFR4, wherein the N-terminal of LFR4 is directly or indirectly connected to the C-terminal of LCDR3, and the LFR4 includes an amino acid sequence shown in SEQ ID NO: 36 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 36.

In some embodiments, where the VL includes framework regions LFR1, LFR2, LFR3 and LFR4, the C-terminal of LFR1 is directly or indirectly connected to the N-terminal of LCDR1, the N-terminal of LFR2 is directly or indirectly connected to the C-terminal of LCDR1, and the C-terminal of LFR2 is directly or indirectly connected to the N-terminal of LCDR2, the N-terminal of LFR3 is directly or indirectly connected to the C-terminal of LCDR2, and the C-terminal of LFR3 is directly or indirectly connected to the N-terminal of LCDR3, the N-terminal of LFR4 is directly or indirectly connected to the C-terminal of LCDR3; among them, the LFR1 includes an amino acid sequence shown in SEQ ID NO: 33 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 33, the LFR2 includes an amino acid sequence shown in SEQ ID NO: 34 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 34, the LFR3 includes an amino acid sequence shown in SEQ ID NO: 35 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 35, and the LFR4 includes an amino acid sequence shown in SEQ ID NO: 36 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 36.

In some embodiments, where the anti PD-L1 antibody includes VL, and the VL includes an amino acid sequence shown in SEQ ID NO: 37 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 37.

In some embodiments, where the anti PD-L1 antibody includes VL, and the VL includes an amino acid sequence shown in SEQ ID NO:38, SEQ ID NO:39 or SEQ ID NO:40 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:38, SEQ ID NO:39 or SEQ ID NO: 40.

In some embodiments, where the anti PD-L1 antibody includes VH and VL, the VH includes an amino acid sequence shown in SEQ ID NO: 25 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 25, and the VL includes an amino acid sequence shown in SEQ ID NO: 37 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 37.

For example, where the anti PD-L1 antibody can include VH and VL, the VH can include an amino acid sequence shown in SEQ ID NO: 25, and the VL can include an amino acid sequence shown in SEQ ID NO: 37.

For example, the pharmaceutical combination can include: 1) anti PD-L1 antibody, wherein the anti PD-L1 antibody can include VH and VL, the VH can include an amino acid sequence shown in SEQ ID NO: 25, and the VL can include an amino acid sequence shown in SEQ ID NO: 37; 2) the imidazole quinoline derivative, where the imidazole quinoline derivative may be the Imiquimod or the derivatives thereof, and a combination thereof.

In some embodiments, where the anti PD-L1 antibody includes VH and VL, the VH includes an amino acid sequence shown in SEQ ID NO: 25 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 25, and the VL includes an amino acid sequence shown in SEQ ID NO:38, SEQ ID NO:39 or SEQ ID NO:40 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:38, SEQ ID NO:39 or SEQ ID NO:40.

For example, where the anti PD-L1 antibody can include VH and VL, the VH can include an amino acid sequence shown in SEQ ID NO: 25, and the VL can include an amino acid sequence shown in SEQ ID NO:38, SEQ ID NO:39 or SEQ ID NO:40.

For example, the pharmaceutical combination can include: 1) anti PD-L1 antibody, wherein the anti PD-L1 antibody can include VH and VL, the VH can include an amino acid sequence shown in SEQ ID NO: 25, and the VL can include an amino acid sequence shown in SEQ ID NO:38, SEQ ID NO: 39 or SEQ ID NO:40; 2) the imidazole quinoline derivative, where the imidazole quinoline derivative may be the Imiquimod or the derivatives thereof, and a combination thereof.

In some embodiments, where the anti PD-L1 antibody includes LC, and the LC includes an amino acid sequence shown in SEQ ID NO: 41 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 41.

In some embodiments, where the anti PD-L1 antibody includes LC, and the LC includes an amino acid sequence shown in SEQ ID NO:42, SEQ ID NO:43 or SEQ ID NO:44 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:42, SEQ ID NO:43 or SEQ ID NO: 44.

In some embodiments, where the anti PD-L1 antibody includes HC and LC, the HC includes an amino acid sequence shown in SEQ ID NO: 26 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 26, and the LC includes an amino acid sequence shown in SEQ ID NO: 41 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 41.

For example, where the anti PD-L1 antibody can include HC and LC, the HC can include an amino acid sequence shown in SEQ ID NO: 26, and the LC can include an amino acid sequence shown in SEQ ID NO: 41.

For example, the pharmaceutical combination can include: 1) anti PD-L1 antibody, wherein the anti PD-L1 antibody can include HC and LC, the HC can include an amino acid sequence shown in

SEQ ID NO: 26, and the LC can include an amino acid sequence shown in SEQ ID NO: 41; 2) the imidazole quinoline derivative, where the imidazole quinoline derivative may be the Imiquimod or the derivatives thereof, and a combination thereof.

In some embodiments, where the anti PD-L1 antibody includes HC and LC, the HC includes an amino acid sequence shown in SEQ ID NO: 26 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO: 26, and the LC includes an amino acid sequence shown in SEQ ID NO:42, SEQ ID NO:43 or SEQ ID NO:44 or an amino acid sequence having at least about 70%, about 75%, about 80%, about 85%, about 90%, about 91%, about 92%, about 93%, about 94%, about 95%, about 96%, about 97%, about 98%, or about 99% sequence identity to the amino acid sequence shown in SEQ ID NO:42, SEQ ID NO:43 or SEQ ID NO:44.

For example, where the anti PD-L1 antibody can include HC and LC, the HC can include an amino acid sequence shown in SEQ ID NO: 26, and the LC can include an amino acid sequence shown in SEQ ID NO:42, SEQ ID NO:43 or SEQ ID NO:44.

For example, the pharmaceutical combination can include: 1) anti PD-L1 antibody, wherein the anti PD-L1 antibody can include HC and LC, the HC can include an amino acid sequence shown in SEQ ID NO: 26, and the LC can include an amino acid sequence shown in SEQ ID NO:42, SEQ ID NO: 43 or SEQ ID NO:44; 2) the imidazole quinoline derivative, where the imidazole quinoline derivative may be the Imiquimod or the derivatives thereof, and a combination thereof.

In some embodiments, i) the PD-1 inhibitor and/or PD-L1 inhibitor in the pharmaceutical combination are not mixed with ii) the TLR agonist to each other in the pharmaceutical combination, i.e., i) the PD-1 inhibitor and/or ii) the PD-L1 inhibitor and TLR agonist are located in separate dosage forms.

In some embodiments, i) the PD-1 inhibitor and/or PD-L1 inhibitor, and ii) the TLR agonist are present in the pharmaceutical combination in a single dosage form.

In some embodiments, where the pharmaceutical combination is formulated into a pharmaceutical composition (e.g., a composite formulation). This pharmaceutical combination can be directly injected into large tumors without affecting normal (surrounding) tissues, enabling the killing of cancer cells, preventing or delaying the growth of malignant mass (e.g., making the tumor smaller or regression), and enabling advanced cancer patients to live with the tumor in a similar manner to those living with parasites. When a pharmaceutical combination is injected into a tumor, the drug can flow along blood vessels or lymphatic vessels to the metastatic lesion, and it will kill the metastatic cells. The injection of pharmaceutical combination into the tumor results in minimal trauma to the patient and can be repeated multiple times a month, for example. Direct injection can also be applied simultaneously to primary tumors and secondary tumors to which cancer has metastasized.

In some embodiments, where the pharmaceutical composition includes a PD-1 inhibitor or a PD-L1 inhibitor, and a TLR agonist.

For example, the pharmaceutical composition can include a PD-1 inhibitor and a TLR agonist. For example, the pharmaceutical composition can include an anti PD-1 antibody or antigen-binding fragments thereof and the imidazole quinoline derivative.

For example, the pharmaceutical composition can include a PD-L1 inhibitor and a TLR agonist. Yet for example, the pharmaceutical composition can include an anti PD-L1 antibody or antigen-binding fragments thereof and the imidazole quinoline derivative.

In some embodiments, where the TLR agonist is present in an amount of about 0.0001 mg/kg to about 200 mg/kg. For example, the TLR agonist can be present in the combination described herein in an amount relative to the subject's body weight (i.e., mg/kg). In some cases, the TLR agonist is present in an amount equivalent to about 0.0001 mg/kg to about 200 mg/kg, 0.001 mg/kg to about 200 mg/kg, 0.01 mg/kg to about 200 mg/kg, 0.01 mg/kg to about 150 mg/kg, 0 01 mg/kg to about 100 mg/kg, 0.01 mg/kg to about 50 mg/kg, 0.01 mg/kg to about 25 mg/kg, 0.01 mg/kg to about 10 mg/kg or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg or 0.5 mg/kg to about 5 mg/kg. In other cases, the TLR agonist is present in an amount equivalent to about 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg.

Yet for example, the TLR agonist can be present in the combination in an amount of about 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg or 2000 mg. The TLR agonist can be present in the combination in an amount of about 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 mg to about 2000 mg.

Yet for example, the TLR agonist can be present in the combination in an amount of about 0.1 mg/mL, 0.5 mg/mL, 1 mg/mL, 2 mg/ml, 3 mg/mL, 4 mg/mL, 5 mg/mL, 6 mg/mL, 7 mg/mL, 8 mg/mL, 9 mg/mL, 10 mg/mL, 15 mg/mL, 20 mg/mL, 25 mg/mL, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In some embodiments, the TLR agonist can be present in the combination in an amount of about 1 mg/ml to about 10 mg/mL, 5 mg/ml to about 10 mg/mL, 5 mg/ml to about 15 mg/mL, 10 mg/ml to about 25 mg/mL, 20 mg/mL to about 30 mg/mL, 25 mg/ml to about 50 mg/ml or 50 mg/ml to about 100 mg/mL.

In some embodiments, where the PD-1 inhibitor or PD-L1 inhibitor (e.g., the anti PD-1/PD-L1 antibody) is present in an amount of about 0.0001 mg/kg to about 200 mg/kg. For example, the PD-1 inhibitor or PD-L1 inhibitor can be present in the combination described herein in an amount relative to the subject's body weight (i.e., mg/kg). In some cases, the PD-1 inhibitor or PD-L1 inhibitor is present in an amount equivalent to about 0.0001 mg/kg to about 200 mg/kg, 0.001 mg/kg to about 200 mg/kg, 0.01 mg/kg to about 200 mg/kg, 0.01 mg/kg to about 150 mg/kg, 0 01 mg/kg to about 100 mg/kg, 0.01 mg/kg to about 50 mg/kg, 0.01 mg/kg to about 25 mg/kg, 0.01 mg/kg to about 10 mg/kg or 0.01 mg/kg to about 5 mg/kg, 0.05 mg/kg to about 200 mg/kg, 0.05 mg/kg to about 150 mg/kg, 0.05 mg/kg to about 100 mg/kg, 0.05 mg/kg to about 50 mg/kg, 0.05 mg/kg to about 25 mg/kg, 0.05 mg/kg to about 10 mg/kg or 0.05 mg/kg to about 5 mg/kg, 0.5 mg/kg to about 200 mg/kg, 0.5 mg/kg to about 150 mg/kg, 0.5 mg/kg to about 100 mg/kg, 0.5 mg/kg to about 50 mg/kg, 0.5 mg/kg to about 25 mg/kg, 0.5 mg/kg to about 10 mg/kg or 0.5 mg/kg to about 5 mg/kg. In other cases, the PD-1 inhibitor or PD-L1 inhibitor is present in an amount equivalent to about 1 mg/kg to about 200 mg/kg, 1 mg/kg to about 150 mg/kg, 1 mg/kg to about 100 mg/kg, 1 mg/kg to about 50 mg/kg, 1 mg/kg to about 25 mg/kg, 1 mg/kg to about 10 mg/kg, or 1 mg/kg to about 5 mg/kg.

Yet for example, the anti PD-1/PD-L1 antibody can be present in the combination in an amount of about 1 mg, 5 mg, 10 mg, 15 mg, 20 mg, 25 mg, 30 mg, 40 mg, 50 mg, 60 mg, 70 mg, 75 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, or 2000 mg. The anti PD-1/PD-L1 antibody can be present in the combination in an amount of about 1 mg to about 10 mg, 10 mg to about 20 mg, 25 mg to about 50 mg, 30 mg to about 60 mg, 40 mg to about 50 mg, 50 mg to about 100 mg, 75 mg to about 150 mg, 100 mg to about 200 mg, 200 mg to about 500 mg, 500 mg to about 1000 mg, 1000 mg to about 1200 mg, 1000 mg to about 1500 mg, 1200 mg to about 1500 mg, or 1500 mg to about 2000 mg.

Yet for example, the anti PD-1/PD-L1 antibody can be present in the combination in an amount of about 0.1 mg/mL, 0.5 mg/mL, 1 mg/mL, 2 mg/ml, 3 mg/mL, 4 mg/mL, 5 mg/ml, 6 mg/mL, 7 mg/mL, 8 mg/mL, 9 mg/mL, 10 mg/mL, 15 mg/mL, 20 mg/mL, 25 mg/ml, 30 mg/mL, 40 mg/mL, 50 mg/mL, 60 mg/mL, 70 mg/mL, 80 mg/mL, 90 mg/mL, 100 mg/mL, 150 mg/mL, 200 mg/mL, 250 mg/mL, 300 mg/mL, 400 mg/mL, or 500 mg/mL. In some embodiments, the anti PD-1/PD-L1 antibody can be present in the combination in an amount of about 1 mg/ml to about 10 mg/mL, 5 mg/mL to about 10 mg/mL, 5 mg/mL to about 15 mg/mL, 10 mg/ml to about 25 mg/mL, 20 mg/mL to about 30 mg/mL, 25 mg/ml to about 50 mg/mL or 50 mg/ml to about 100 mg/mL.

In some embodiments, the TLR agonist can be provided in a synergistic amount with the amount of the PD-1/PD-L1 inhibitor. The administration dosage will undoubtedly vary with known factors, such as the pharmacokinetic properties of a particular agent and mode and route of administration thereof; age, physical condition, and weight of the recipient; nature and extent of symptoms, type of concurrent treatment, frequency of treatment, and expected effect.

In some embodiments, where the pharmaceutical composition further includes one or more pharmaceutically acceptable carriers.

For example, the example of the pharmaceutically acceptable carrier may include one or more of the following: water, saline, phosphate buffered saline, dextrose, glycerol, ethanol, etc., and a combination thereof. In some embodiments, the composition may include an isotonic agent, such as sugar, polyol (such as mannitol and sorbitol), or sodium chloride. The pharmaceutically acceptable carrier may further include a small amount of an auxiliary substance that enhances the shelf life or effectiveness of a binding protein or partial binding protein, for example, a wetting agent or an emulsifying agent, a preservative, or a buffer agent.

In another aspect, the disclosure also provides a use of the aforementioned pharmaceutical combination in the preparation of drugs for the treatment of neoplastic diseases.

In some embodiments, where the neoplastic diseases include tumors and/or wart diseases.

In some embodiments, the tumors (or cancers) include but are not limited to: hepatocellular carcinoma, hepatic metastasis cancer, advanced hepatocellular carcinoma, pancreatic carcinoma, adenocarcinoma, mastocytoma or mast cell tumor, ovarian carcinoma, non-small-cell lung carcinoma, small-cell lung carcinoma, melanoma, retinoblastoma, breast neoplasms, colorectal carcinoma, histiocytosarcoma, brain neoplasms, astrocytoma, glioblastoma, neuroma, neuroblastoma, colorectal carcinoma, cervical carcinoma, sarcoma, prostatic neoplams, bladder neoplasms, reticuloendothelium neoplasms, nephroblastoma, ovarian carcinoma, osteocarcinoma, osteosarcoma, renal carcinoma, or head and neck cancer, oral carcinoma, laryngocarcinoma, or oropharyngeal carcinoma, breast carcinoma, genitourinary tract carcinoma, lung carcinoma, gastrointestinal carcinoma, epidermoid carcinoma, ormelanoma.

In another aspect, the disclosure also provides the aforementioned pharmaceutical combination for use in the treatment of neoplastic diseases.

In another aspect, the disclosure also provides a drug including the aforementioned pharmaceutical combination for treating neoplastic diseases.

In another aspect, the disclosure also provides a method for treating neoplastic diseases, which includes administering an effective amount of the aforementioned pharmaceutical combination to a subject in need thereof.

In some embodiments, where the subject suffers from neoplasm.

In some embodiments, where the neoplasm of mammals is located at a site selected from the group consisting of:

brain, head, eyes, nasopharynx, mouth, tongue, neck, thyroid, gastrointestinal system, liver, pancreas, gall bladder, lungs, respiratory system, genitourinary system, kidney, bladder, breast, lymphatic system, cardiovascular system, nervous system, skin, thoracic cavity, pleura, musculoskeletal system, and abdomen, which has primary or secondary natures.

In some embodiments, where the neoplasm includes tumors and/or warts.

In some embodiments, where the administration includes local, intraneoplastic (e.g., in tumors or warts), or systemic administrations. For example, using intratumoral or intraverrucous injection allows for both less trauma to the patient and the killing of cancer cells instead of normal cells. The direct injection of pharmaceutical combinations into malignant tumors also greatly reduces or eliminates many common side effects.

In some embodiments, where the administration includes intravenous injection, intravenous instillation, intramuscular injection, subcutaneous injection, and/or intraneoplastic injection.

In some embodiments, the tumor includes:
(i) superficial malignant diseases of skin, eyes, tongue, mouth, thyroid, breast, cervix, uterus, anus, prostate, vagina, osteosarcoma, urethral carcinoma, penis, testicles, and epididymis, and the pharmaceutical combination is directly injected into the tumor body without dilution using a syringe; or
(ii) cancer of nasopharynx, and the pharmaceutical combination is injected into the tumor body through a nasopharyngoscopy using a syringe or a needle; or
(iii) cancers of liver, kidney, and gallbladder, and the pharmaceutical combination is injected into the tumor body through the skin with the assistance of ultrasound using a syringe, or into the tumor body through a pore formed in the patients' abdominal wall during laparoscopic surgery; or
(iv) cancers of ovarian, fallopian tube, pancreas, lymph node metastasis or direct peritoneal invasion of the abdominal cavity, and abdominal lymphoma, and the pharmaceutical combination is injected into the tumor body through a pore formed in the patients' abdominal wall during laparoscopic surgery using a syringe; or
(v) cancers or sarcoma of esophagus, stomach, duodenum, and small intestine, and the pharmaceutical combination is injected into the tumor body through colonoscopy using a needle, or through a pore formed in the patients' abdominal wall during laparoscopic surgery through a long syringe, or through a pore formed in the patients' chest wall during thoracoscopic surgery;
(vi) cancers or sarcoma of large intestine and rectum, and the pharmaceutical combination is injected into the tumor body through colonoscopy using a needle, or through a pore formed in the patients' abdominal wall during laparoscopic surgery using a syringe; or
(vii) cancers or sarcoma of lung and trachea, and the pharmaceutical combination is injected into the tumor body through a fiberbronchoscopy using a needle; or
(viii) cancer of lung, and the pharmaceutical combination is injected with a syringe using ultrasound, X-ray, CT scan, or MR scan, or through a pore formed in the patients' chest wall during thoracoscopic surgery; or
(ix) cancer or sarcoma of bladder, and the pharmaceutical combination is injected into the tumor body through cystoscopy using a needle, or through a pore formed in the patients' abdominal wall during laparoscopic surgery; or
(x) cancer or sarcoma of uterus, and the injectable formulation of the pharmaceutical combination is injected into the tumor body using a syringe or hysteroscopic needle; or injected through a pore formed in the patients' abdominal wall during laparoscopic surgery; or
(xi) cancer or sarcoma of nasopharynx and larynx, and the pharmaceutical combination is injected into the tumor body through laryngoscopy using a needle; or
(xii) cancer of brain, and the pharmaceutical combination is injected into the tumor body with a syringe or fibroscopic needle by use of X-ray, CT scan, or MR scan after drilling a pore in the corresponding skull; or
(xiii) malignant lymphoma or lymph nodes with metastasis, and the pharmaceutical combination is injected into the tumor body through the patients' skin using a needle, or through a pore formed in the patients' abdominal wall during laparoscopic surgery, or through a pore formed in the patients' chest wall during thoracoscopic surgery.

In some embodiments, the neoplasm includes warts, and the pharmaceutical combination is injected into the wart body through the patients' skin using a needle.

In some embodiments, where i) the PD-1 inhibitor or PD-L1 inhibitor, and ii) the TLR agonist in the pharmaceutical combination are administered by use of the same or different administration routes.

In some embodiments, it includes injection of the TLR agonist into the neoplasm.

In some embodiments, it also includes injection or systemic infusion (e.g., intravenous injection and intravenous instillation) of the PD-1 inhibitor or PD-L1 inhibitor into the neoplasm.

In some embodiments, it includes injection of i) the PD-1 inhibitor or PD-L1 inhibitor, and ii) the TLR agonist in the pharmaceutical combination into the neoplasm.

In some embodiments, where i) the PD-1 inhibitor or PD-L1 inhibitor, and ii) the TLR agonist in the pharmaceutical combination are administered simultaneously or at different times.

In some embodiments, where the PD-1 inhibitor or PD-L1 inhibitor is administered before and/or after the administration of the TLR agonist.

For example, the PD-1/PD-L1 inhibitor is administered at least 5 minutes, 10 minutes, 20 minutes, 40 minutes, 1 hour, 2 hours, 4 hours, 8 hours, 12 hours, 16 hours, 1 day, 2 days, 3 days, 4 days, 5 days, 6 days, 7 days or 2 weeks before and/or after the administration of the TLR agonist.

In some embodiments, i) the PD-1 inhibitor or PD-L1 inhibitor, and ii) the TLR agonist in the pharmaceutical combination are simultaneously administered by way of intraneoplastic injection.

In some embodiments, i) the PD-1 inhibitor or PD-L1 inhibitor, and ii) the TLR agonist in the pharmaceutical combination are simultaneously administered by way of intraneoplastic injection, and i) the PD-1 inhibitor or PD-L1 inhibitor and ii) the TLR agonist are present in a same dosage form.

In some embodiments, where the PD-1 inhibitor or PD-L1 inhibitor in the pharmaceutical combination is administered by way of intravenous injection, the TLR agonist is simultaneously administered by way of intraneoplastic injection, and the PD-1 inhibitor or PD-L1 inhibitor and the TLR agonist are present in separate dosage forms.

In another aspect, the disclosure provides a pharmaceutical kit including the aforementioned pharmaceutical combination.

In some embodiments, each component of the pharmaceutical combination in the pharmaceutical kit can be provided in separated individual containers. Alternatively, the components of the pharmaceutical combination described herein can be provided in a single container. In such cases, the container can be a container prepared for disclosure to patients in need thereof, such as IV bags, ampoules, or syringes. In some embodiments, the TLR agonist in the pharmaceutical kit is formulated for administration through intratumoral or intraverrucous injection. The PD-1/PD-L1 inhibitor can be provided in the form of powder (e.g., lyophilized powder) or parenteral solution, for example. In some embodiments, the PD-1/PD-L1 inhibitors and the TLR agonist are prepared as composite preparations, and are used to being administered by means of intratumoral or intra-wart injection. In some implementations, the composite preparation is prepared as a liquid preparation. For example, the composite preparation is provided in the form of a stable solution. For another example, the composite preparation is provided in a dosage form that may be directly injected (i.e., without being diluted before use). In some other implementations, the composite preparation is prepared as a solid preparation (for example, in a freeze-drying form).

The content of the pharmaceutical kit described herein can be provided in sterile form. The pharmaceutical kit and content therein can be provided in the form of ready-to-use to a subject in need thereof. In such cases, the components in the pharmaceutical kit are provided as a formulation and optionally provided in an administration device, so that the administration requires almost no further action by the user. In the case where the pharmaceutical kit includes an administration device, such devices include devices known and understood by those skilled in the art for the administration routes described herein, such as but not limited to syringes, pumps, bags, cups, inhalators, droppers, patches, creams, or injectors.

The pharmaceutical kit described herein may also include an instruction, which contains information about, for example, usage, dosage, administration, contraindications, and/or warnings about the use of such drugs.

Without being limited by any theory, the examples hereinafter are only intended to illustrate the pharmaceutical combinations and uses of the disclosure, rather than limiting the scope of invention of the disclosure.

### EXAMPLES

Animal species, strain, gender, weight, sources, certificate of conformity

C57/BL6JNifdc mouse, female, body weight of 17-22 g, aged 6-8 weeks, SPF grade, purchased from Beijing Vital River Laboratory Animal Technology Co., Ltd. [Experimental Animal Quality Certificate No.: SCXK (Beijing) 2016-0006].

### Feeding Conditions

All mice were free to forage and drink water, and were raised at room temperature (25±2)° C. The feed and water were subjected to high-pressure sterilization treatment, and the entire experimental feeding process was SPF grade.

### Tumor Cell Strains

Mouse colorectal carcinoma cell strain MC38 and CT26, mouse lung carcinoma Lewis tumor strain LLC1, mouse melanoma cell strain B16, mouse prostatic carcinoma cell strain RM-1, TRAMP-C1, mouse breast carcinoma cell strains 4T1, NAFA, and MET-1 etc.

### Establishment of Subcutaneous Xenograft Tumor Model

Tumor cell strains were selected from the group consisting of: mouse colorectal carcinoma cell strains CT26 and MC38, mouse lung carcinoma Lewis tumor strain LLC1, mouse melanoma cell strain B16, mouse prostatic carcinoma cell strain RM-1, TRAMP-C1, mouse breast carcinoma cell strains 4T1, NAFA, and MET-1, etc.

Tumor cells were cultured and passaged, cells were collected at the logarithmic phase, and formulated into a concentration of (1.0×10⁷) per milliliter of cell suspension with 0.1 ml (with a cell number of 1.0×10⁶) being injected into the right flank of the mice, the tumor grew to a diameter of about 5 mm within about 10 days, and the model was successfully established, they were randomly divided into 8 groups.

### Drug Evaluation Indicators

Trend of changes in body weight and tumor volume of mice, survival rate of mice, and inhibitory rate of anatomical tumor weight.

Tumor weight inhibitory rate=[1−average tumor weight of experimental group (G2/G3/G4)/average tumor weight of negative control group G1)]×100%.

### Statistical Analysis

The data were represented as x±s, and processed by SPSS10.0 software, using one-way ANOVA. Differences in data among groups (P value) were processed through statistical processing, and there was a significant difference among groups at P<0.05.

### Example 1

The anti-tumor effect of a pharmaceutical combination composed of PD-L1 inhibitor RB0005 and a TLR7/TLR8 agonist Imiquimod (LPG2005) was tested using a xenograft tumor model, i.e., the inhibitory effect on the growth of subcutaneous xenograft tumor in mice.

### Main Steps of the Test

### 1.1 Xenograft Tumor Model in Mice

After successfully establishing a subcutaneous xenograft tumor model of colorectal carcinoma MC38 in mice, it was randomly grouped with 6-8 mice per group. The mice were administered once every two days, with a total of three times followed by observation until 15 days.

| | Administration | Administration | Administration |
|---|---|---|---|
| Groups | Method | dosage | frequency |
| G1 Negative | Intratumoral | Solvent | Administered |
| Control | injection(i.t.) | | every other day |
| | | | with a total of 4 |
| | | | times |
| G2 single drug | Intratumoral | PD-L1 monoclonal | Administered |
| group | injection | antibody | every other day |
| | | RB0005, 1 mg/kg | with a total of 4 |
| | | | times |
| G3 single drug | Intratumoral | Imiquimodsingle | Administered |
| group | injection | drug, 5 | every other day |
| | | mg/kg | with a total of 4 |
| | | | times |
| G4 trial group | Intratumoral | PD-L1 monoclonal | Administered |
| (Single | injection | antibody | every other day |
| dosage form) | | RB0005, 1 mg/kg + | with a total of 4 |
| | | Imiquimod, | times |
| | | 5 mg/kg; | |

### 1.2 Results

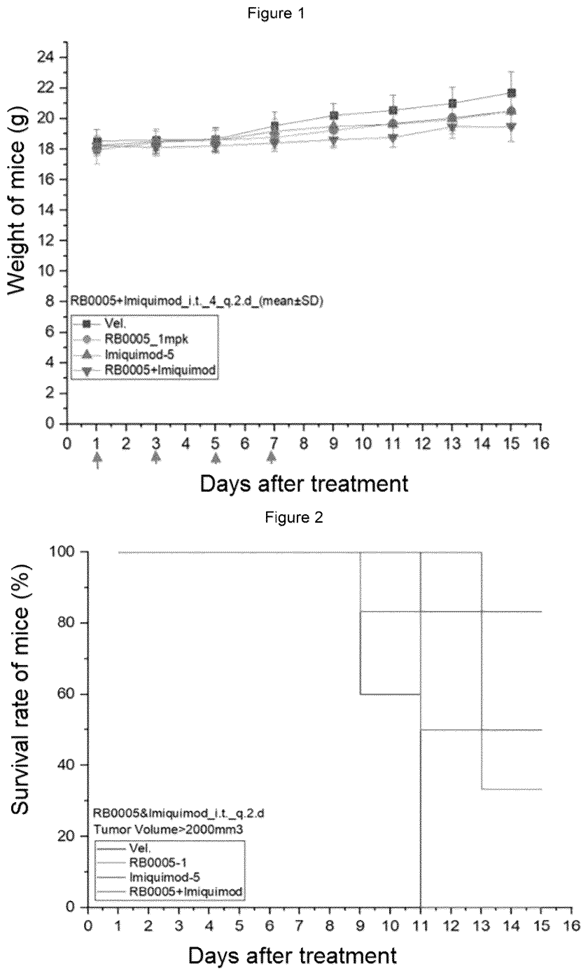

FIGS. 1-4 show that both the novel pharmaceutical combination and each single drug can significantly inhibit the growth of tumor, and the anatomical tumor weight is significantly lower than the negative control group (P<0.01, P<0.001). The novel pharmaceutical combinations have better efficacy than Imiquimod or PD-L1 antibody RB0005 alone, indicating that the novel pharmaceutical combinations have greater advantages.

As shown in FIG. 2, the mice in solvent control group began to experience “death” on day 9 (when the tumor volume exceeded 2000 mm³ in a single mouse, the mouse could be considered as death during the statistical analysis of survival rate in mouse (which was not excluded in the analysis of tumor volume change curve), at Day 11 after initiation of administration, the survival rate of the mice was 0; and the RB0005 single drug group had a 33.3% survival rate in mice as of the end. The Imiquimod single drug group had a 50% survival rate in mice. A single preparation group had an 83.3% survival rate in mice, where the tumor disappeared in 1 mouse in the group, and the tumor volumes of 2 mice continued to decrease (tumor volume <100mm³).

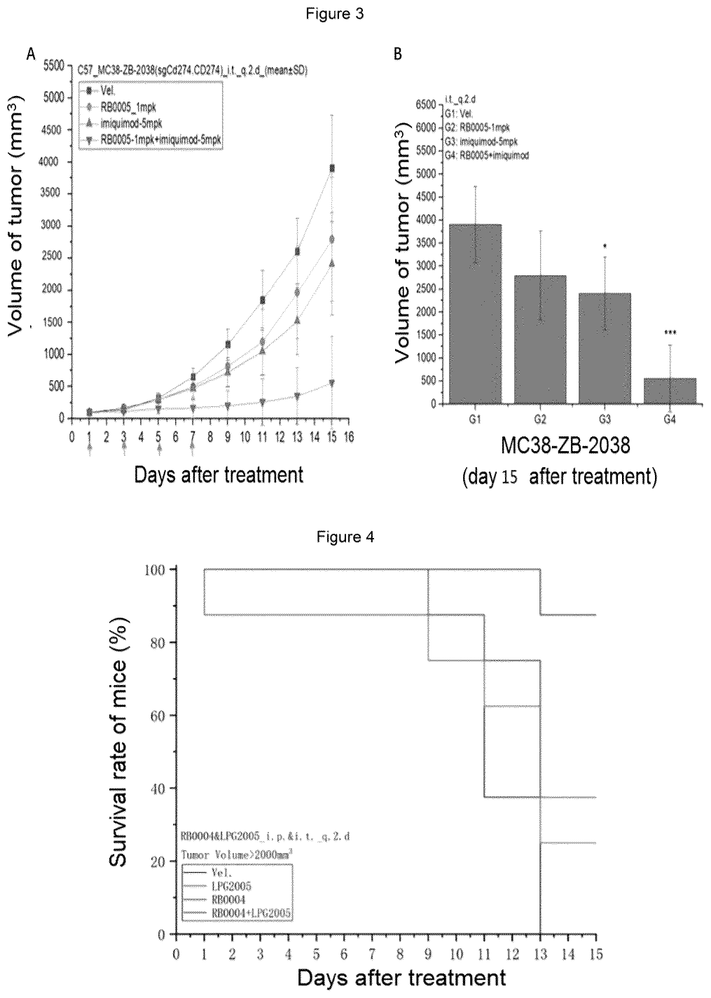

As shown in FIGS. 3A-3B, the tumor weights of the mice in the single preparation group (G4) were significantly reduced, and a tumor weight inhibition rate was 87.9%, which was statistically significant (P<0.001).

### Example 2

The anti-tumor effect of a pharmaceutical combination composed of PD-1 inhibitor RB0004 and a TLR7/TLR8 agonist Imiquimod was tested using a xenograft tumor model, i.e., the inhibitory effect on the growth of subcutaneous xenograft tumor in mice.

### 2.1 Xenograft Tumor Model in Mice

After successfully establishing a humanized PD-1 subcutaneous xenograft tumor model of colorectal carcinoma MC38 in mice, they were randomly grouped with 8 mice per group and administered once every two days.

| | Administration | Administration | Administration |
|---|---|---|---|
| Groups | Method | dosage | frequency |
| G1 Negative | Intratumoral | Solvent | Administering |
| Control | injection | | every other day |
| | | | with a total of 3 |
| | | | times |
| G2 single | Intratumoral | Imiquimodsingle | Administering |
| drug group | injection | drug, 5 mg/kg | every other day |
| | | | with a total of 3 |
| | | | times |
| G3 single | Intraperitoneal | PD-1 monoclonal | Administering |
| drug group | injection (i.p.) | antibody | every other day |
| | | RB0004, 10 mg/kg | with a total of 3 |
| | | | times |
| G4 trial | Intratumoral | Imiquimodsingle | Administering |
| group | injection | drug, 5 mg/kg | every other day |
| (separate | Intraperitoneal | PD-1 monoclonal | with a total of 3 |
| dosage | injection | antibody | times |
| form) | | RB0004, 10 mg/kg | |

### 2.2 Results

Both the novel pharmaceutical combination (separate dosage form) and each single drug can inhibit the growth of tumor, and the anatomical tumor weight is significantly lower than the negative control group (P<0.05, P<0.001). The novel pharmaceutical combinations have better efficacy than Imiquimod or PD-1 antibody RB0004 alone, indicating that the novel pharmaceutical combinations have greater advantages.

As shown in FIG. 4, at Day 13 after initiation of administration in the solvent control group, the survival rate of the mice was 0; and the RB0004 single drug group had 37.5% survival rate in mice as of the end. An Imiquimod group had 40% survival rate in mice. A combined administration group (G4) had 87.5% survival rate in mice, where the tumor volume of 1 mouse continued to decrease (tumor volume <100mm³).

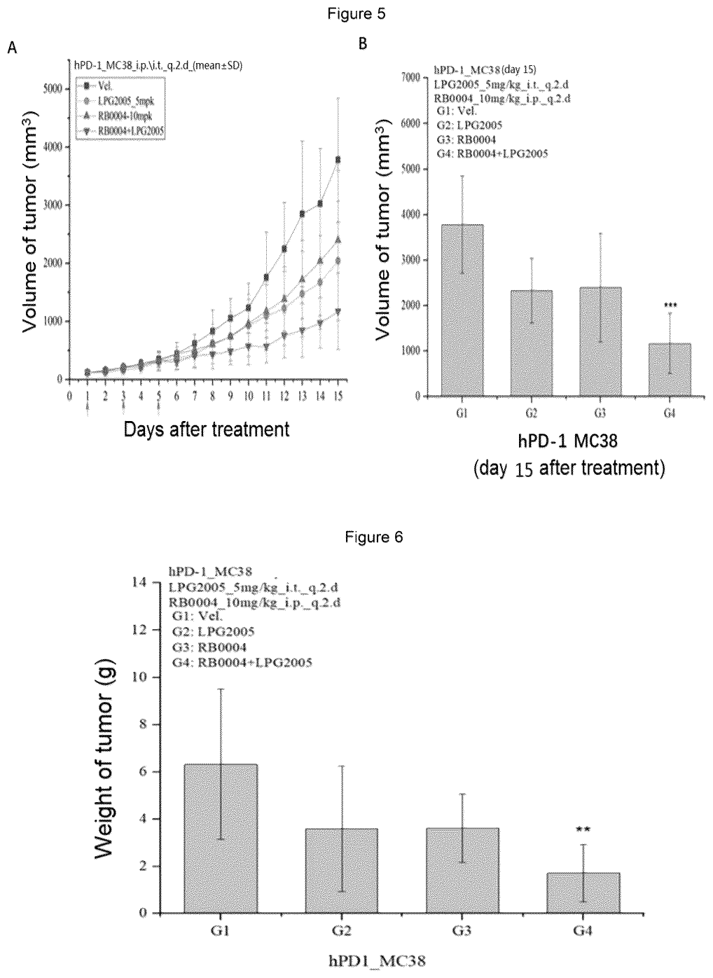

As shown in FIG. 5, the tumor volumes of the mice in the solvent control group gradually increased over time; in the RB0004 single drug group and a LPG2005 single drug group, the growth of the tumor volume was slower than that in the solvent control group; and after the administration of the combined administration group (G4), the growth of the tumor volume was significantly slowed down, with a statistically significant difference compared to the control group (P<0.001).

As shown in FIG. 6 and Table 1, the tumor weights of the mice in the G4 were significantly reduced, and a tumor weight inhibition rate was 72.99%, which was statistically significant (P<0.001) and CDI=0.83, such that it indicated that the two drugs had a synergistic drug effect.

TABLE 1

| | Tumor | | |
|---|---|---|---|
| Group | weight (g) | IR_{TW} % | CDI |
| G1: Vel | 6.32 | 0.00 | / |
| G2: LPG2005 | 3.62 | 42.71 | / |
| G3: RB0004 | 3.60 | 42.98 | / |
| G4: RB0004 + LPG2005 | 1.71 | 72.99 | 0.83 |

### Example 3

A colorectal cancer MC38 Xenograft tumor model in mice was used to test an anti-tumor effect of a pharmaceutical combination consisting of a PD-L1 inhibitor RB0005 and different doses of TLR7/TLR8 agonists Imiquimod, that is, the inhibitory effect on the growth of subcutaneous xenograft tumor in mice.

### 3.1 Xenograft Tumor Model in Mice

After a subcutaneous xenograft tumor model in mice was successfully established, random grouping was performed, and each group had 8 mice, which were administered once every two days.

| | Administration | Administration | Administration |
|---|---|---|---|
| Group | method | dosage | frequency |
| G1 negative | Intratumoral | Solvent | Administration every |
| control | injection | | other day with a |
| | | | total of 3 times |
| G2 single drug | Intratumoral | PD-L1 monoclonal | Administration every |
| group | injection | antibody | other day with a |
| | | RB0005 1 mg/kg | total of 3 times |
| G3 single drug | Intratumoral | Imiquimod | Administration every |
| group | injection | single drug | other day with a |
| | | 0.5 mg/kg | total of 3 times |
| G4 single drug | Intratumoral | Imiquimod | Administration every |
| group | injection | single drug | other day with a |
| | | 1.58 mg/kg | total of 3 times |
| G5 single drug | Intratumoral | Imiquimod single | Administration every |
| group | injection | drug 5 mg/kg | other day with a |
| | | | total of 3 times |
| G6 trial group | Intratumoral | Imiquimod 0.5 | Administration every |
| (single dosage | injection | mg/kg + PD-L1 | other day with a |
| form) | | monoclonal | total of 3 times |
| | | antibody RB0005 | |
| | | 1 mg/kg | |
| G7 trial group | Intratumoral | Imiquimod 1.58 | Administration every |
| (single dosage | injection | mg/kg + PD-L1 | other day with a |
| form) | | monoclonal | total of 3 times |
| | | antibody RB0005 | |
| | | 1 mg/kg | |
| G8 trial group | Intratumoral | Imiquimod 5 | Administration every |
| (single dosage | injection | mg/kg + PD-L1 | other day with a |
| form) | | monoclonal | total of 3 times |
| | | antibody RB0005 | |
| | | 1 mg/kg | |
| G9 single drug | Local | 5% (w/w) | Administration every |
| group (topical | disclosure | Imiquimod Cream | other day with a |
| cream) | | | total of 3 times |
| G10 trial group | Intratumoral | PD-L1 monoclonal | Administration every |
| (separate | injection | antibody | other day with a |
| dosage | Local | RB0005 1 mg/kg | total of 3 times |
| form) | disclosure | 5% Imiquimod | |
| | | Cream | |

### 3.2 Result

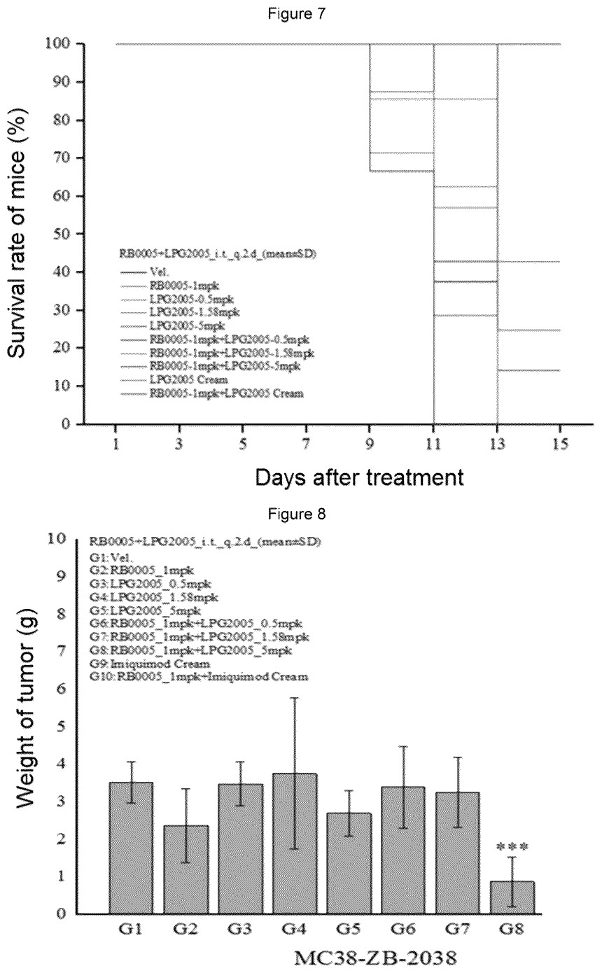

Results: the subcutaneously-Xenograft tumor model in mice was successfully established, in the three single dosage forms, G8 had a significant tumor proliferation inhibition effect, the tumor weight inhibition rates were all above 75% (P<0.001), and there was no tumor inhibition effect (P>0.05) in G6 and G7. Specific results were shown in FIGS. 7-8.

As shown in FIG. 7, the survival rate of the mice in G8 was 100%.

As shown in FIG. 8 and Table 2, the tumor weights of the mice in the G8 were significantly reduced, and a tumor weight inhibition rate was 75.09%, which was statistically significant (P<0.001) and CDI-0.48, such that it indicated that the composite preparation had a synergistic drug effect.

TABLE 2

| | Tumor weight | IR_{TW} | |
|---|---|---|---|
| Group | (g) | % | CDI |
| G1: Vel | 3.53 | 0.00 | / |
| G2: RB0005 | 2.38 | 32.61 | / |
| G5: LPG2005 | 2.70 | 23.35 | / |
| G8: RB0005 + LPG2005 | 0.88 | 75.09 | 0.48 |

### Example 4

A Xenograft tumor model was used to test an anti-tumor effect of a pharmaceutical combination consisting of a PD-L1 inhibitor RB0005 and different doses of TLR7/TLR8 agonists Imiquimod, that is, the inhibitory effect on the growth of subcutaneous xenograft tumor in mice.

### 4.1 Xenograft Tumor Model in Mice

After a subcutaneously-Xenograft tumor model in mice was successfully established, random grouping was performed, and each group had 8 mice, which were administered once every two days.

| | Administration | Administration | Administration |
|---|---|---|---|
| Group | method | dosage | frequency |
| G1 negative | Intratumoral | Solvent | Administration every |
| control | injection | | other day with a |
| | | | total of 3 times |
| G2 single drug | Intratumoral | PD-L1 monoclonal | Administration every |
| group | injection | antibody | other day with a |
| | | RB0005 1 mg/kg | total of 3 times |
| G3 single drug | Intratumoral | Imiquimod single | Administration every |
| group | injection | drug 2 mg/kg | other day with a |
| | | | total of 3 times |
| G4 single drug | Intratumoral | Imiquimod | Administration every |
| group | injection | single drug | other day with a |
| | | 6.32 mg/kg | total of 3 times |
| G5 single drug | Intratumoral | Imiquimod single | Administration every |
| group | injection | drug 20 mg/kg | other day with a |
| | | | total of 3 times |
| G6 trial group | Intratumoral | Imiquimod 2 | Administration every |
| (single dosage | injection | mg/kg + PD-L1 | other day with a |
| form) | | monoclonal | total of 3 times |
| | | antibody RB0005 | |
| | | 1 mg/kg | |
| G7 trial group | Intratumoral | Imiquimod | Administration every |
| (single dosage | injection | single drug 6.32 | other day with a |
| form) | | mg/kg + PD-L1 | total of 3 times |
| | | monoclonal | |
| | | antibody RB0005 | |
| | | 1 mg/kg | |
| G8 trial group | Intratumoral | Imiquimod single | Administration every |
| (single dosage | injection | drug 20 mg/kg + | other day with a |
| form) | | PD-L1 monoclonal | total of 3 times |
| | | antibody | |
| | | RB0005 1 mg/kg | |
| G9 single drug | Local | 5% Imiquimod | Administration every |
| group (topical | disclosure | Cream | other day with a |
| cream) | | | total of 3 times |
| G10 trial group | Intratumoral | PD-L1 monoclonal | Administration every |
| (separate | injection | antibody | other day with a |
| dosage | Local | RB0005 1 mg/kg | total of 3 times |
| form) | disclosure | 5% Imiquimod | |
| | | Cream | |

4.2 Result

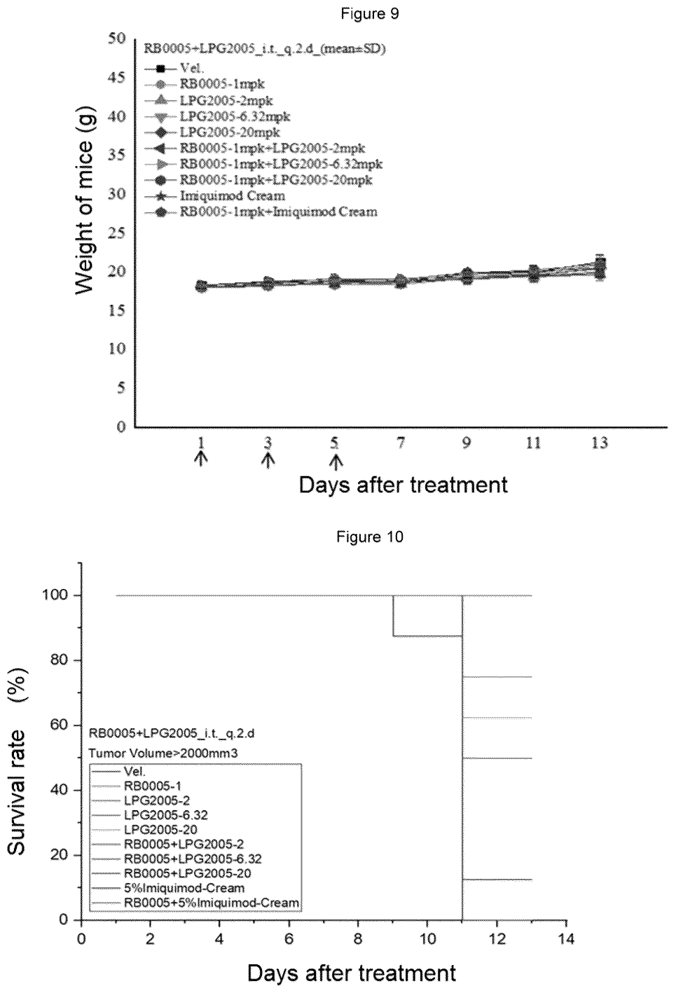

Results: the subcutaneously-Xenograft tumor model in mice was successfully established, in the three single dosage forms, G6 and G7 had significant tumor proliferation inhibition effects, the tumor weight inhibition rates were all above 70% (P<0.001), and there was no tumor inhibition effect (P>0.05) in G8. Specific results were shown in FIGS. 9-11.

As shown in FIG. 10, the survival rate of the mice in the G6, the G7 and the G8 was 100% by the end date of observation. The tumor volumes of 2 mice in G7 continued to decrease, and the tumor volumes of 3 mice in G8 continued to decrease (tumor volume <100mm³). In a RB0005 combined with 5% lmiquimod Cream group (G10), the survival rate of the mice was 100% by the end date of observation. The tumor volumes of 2 mice continued to decrease (tumor volume <100mm³).

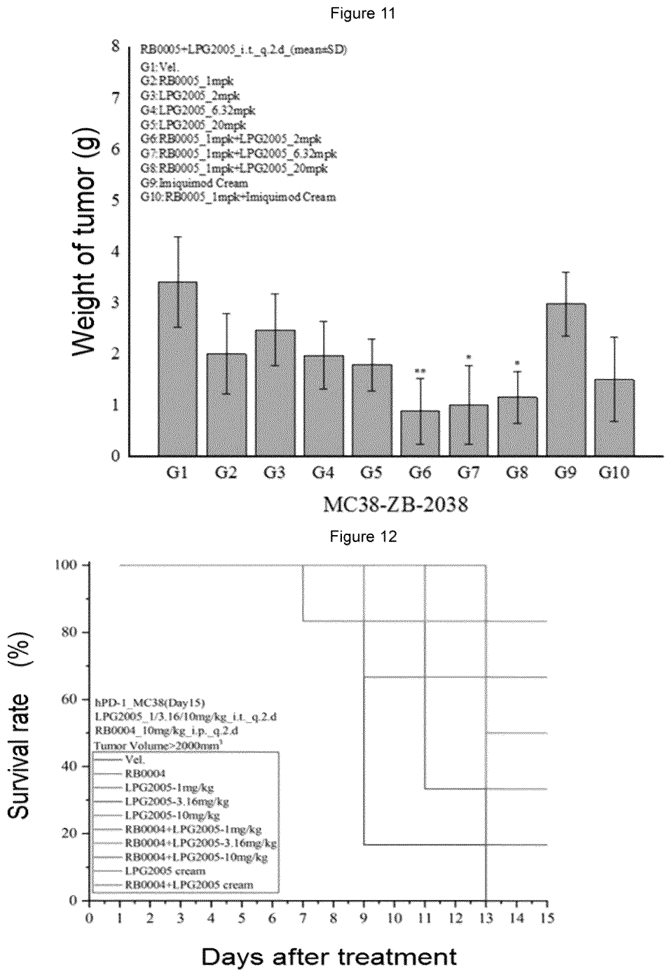

As shown in FIG. 11 and Table 3, the tumor weight inhibition rates in the G6, the G7 and the G8 respectively were 73.87%, 70.47%, and 65.97%, which were statistically significant (P<0.001), but CDI showed that, the G6 and the G7 respectively were 0.61 and 0.86, and the CDI of the G8 was 1.09, such that it indicated that the G6 and the G7 had a synergistic drug effect. The tumor weight inhibition rates in the RB0005 combined with 5% lmiquimod Cream group (G10) were all above 55.77% (P<0.05), CDI=0.86, indicating that the combination of the two had a synergistic drug effect.

TABLE 3

| | Tumor weight | IR_{TW} | |
|---|---|---|---|
| Group | (g) | % | CDI |
| G6 | 1.01 | 73.87 | 0.61 |
| G7 | 1.16 | 70.47 | 0.86 |
| G8 | 2.99 | 65.97 | 1.09 |
| G10 | 1.51 | 55.77 | 0.86 |

### Example 5

A Xenograft tumor model was used to test an anti-tumor effect of a pharmaceutical combination consisting of a PD-1 inhibitor RB0004 and different doses of TLR7/TLR8 agonists Imiquimod, that is, the inhibitory effect on the growth of subcutaneous xenograft tumor in mice.

### 5.1 Xenograft Tumor Model in Mice

After a subcutaneously-Xenograft tumor model in mice was successfully established, random grouping was performed, and each group had 8 mice, which were administered once every two days.

| | Administration | Administration | Administration |
|---|---|---|---|
| Group | method | dosage | frequency |
| G1 negative | Intratumoral | Solvent | Administration |
| control | injection | | every other day with |
| | | | a total of 3 times |
| G2 single drug | Intraperitoneal | PD-1 monoclonal | Administration |
| group | injection | antibody RB0004 | every other day with |
| | | 10 mg/kg | a total of 3 times |
| G3 single drug | Intratumoral | Imiquimod | Administration |
| group | injection | single drug | every other day with |
| | | 1 mg/kg | a total of 3 times |
| G4 single drug | Intratumoral | Imiquimod | Administration |
| group | injection | single drug | every other day with |
| | | 3.16 mg/kg | a total of 3 times |
| G5 single drug | Intratumoral | Imiquimod | Administration |
| group | injection | single drug | every other day with |
| | | 10 mg/kg | a total of 3 times |
| G6 trial group | Intratumoral | Imiquimod | Administration |
| (separate | injection | 1 mg/kg | every other day with |
| dosage | Intraperitoneal | PD-1 monoclonal | a total of 3 times |
| form) | injection | antibody RB0004 | |
| | | 10 mg/kg | |
| G7 trial group | Intratumoral | Imiquimod | Administration |
| (separate | injection | single drug | every other day with |
| dosage | | 3.16 mg/kg | a total of 3 times |
| form) | Intraperitoneal | PD-1 monoclonal | |
| | injection | antibody RB0004 | |
| | | 10 mg/kg | |
| G8 trial group | Intratumoral | Imiquimod | Administration |
| (separate | injection | single drug | every other day with |
| dosage | | 10 mg/kg | a total of 3 times |
| form) | Intraperitoneal | PD-1 monoclonal | |
| | injection | antibody RB0004 | |
| | | 10 mg/kg | |
| G9 single drug | Local | 5% Imiquimod | Administration |
| group (topical | disclosure | Cream | every other day with |
| cream) | | | a total of 3 times |
| G10 trial group | Intraperitoneal | PD-1 monoclonal | Administration |
| (separate | injection | antibody RB0004 | every other day with |
| dosage | | 10 mg/kg | a total of 3 times |
| form) | Local | 5% Imiquimod | |
| | disclosure | Cream | |

### 5.2 Result

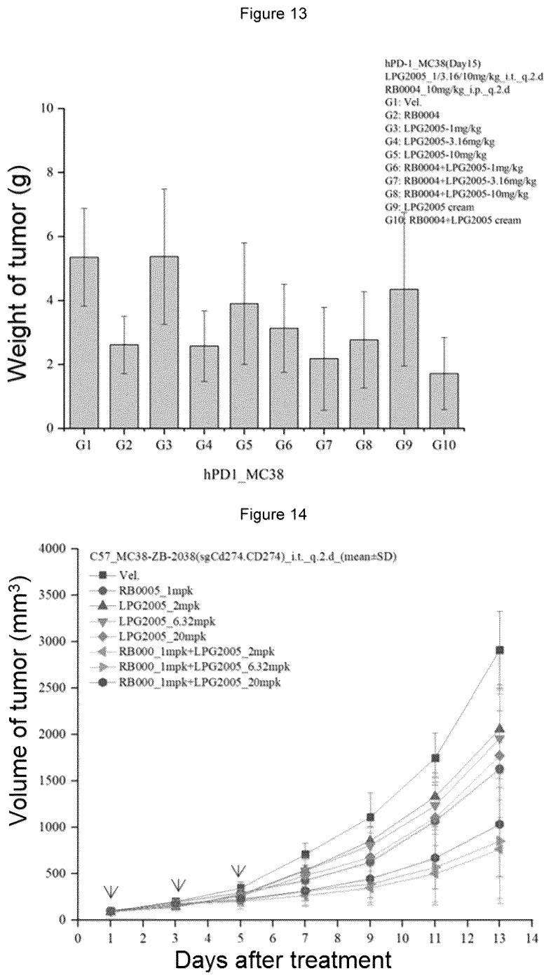

Results: the G7 had a significant tumor inhibition effect (P<0.05), and the tumor weight inhibition rate was 59.25%. Specific results were shown in FIG. 12-FIG. 13.

As shown in FIG. 12, the RB0004 was combined with LPG2005-1, 3.16mpk, the survival rate of the mice was higher than 50% by the end date of observation. In a RB0004 combined with 5% Cream group, the survival rate of the mice was 83.3% by the end date of observation.

As shown in FIG. 13 and Table 4, by comparing the G7 with the control group, the tumor weight inhibition rate was 59.25%, which was statistically significant. In the RB0004 combined with 5% Cream group (G10), the tumor weight inhibition rate was 67.82% and CDI-0.78, indicating that the combination of the two had a synergistic drug effect.

TABLE 4

| | Tumor weight | IR_{TW} | |
|---|---|---|---|
| Group | (g) | % | CDI |
| G10 | 1.72 | 67.82 | 0.775 |

### Example 6

### Anti-Tumor Effect of Pharmaceutical Combination LPG2006 (Single Dosage Form) Consisting of PD-L1 Inhibitor RB0005 and Different Doses of TLR7/TLR8 Agonists Imiquimod

### 6.1 Xenograft Tumor Model in Mice

After a colorectal cancer MC38 humanized CD274 cell subcutaneously-Xenograft tumor model in mice was successfully established, random grouping was performed, and each group had 6-8 mice, which were administered once every two days with a total of 3 times.

### 6.2 Grouping

| | Administration | Administration | Administration |
|---|---|---|---|
| Group | method | dosage | frequency |
| G1 negative | Intratumoral | Solvent | Administration |
| control | injection | | every other day with |
| | | | a total of 3 times |
| G2 single drug | Intratumoral | PD-L1 monoclonal | Administration |
| group | injection | antibody | every other day with |
| | | RB0005, 1 mg/kg | a total of 3 times |
| G3 single drug | Intratumoral | Imiquimod | Administration |
| group | injection | (LPG2005) single | every other day with |
| | | drug 2 mg/kg | a total of 3 times |
| G4 single drug | Intratumoral | Imiquimod | Administration |
| group | injection | single drug | every other day with |
| | | 6.32 mg/kg | a total of 3 times |
| G5 single drug | Intratumoral | Imiquimod | Administration |
| group | injection | single drug | every other day with |
| | | 20 mg/kg | a total of 3 times |
| G6 trial group | Intratumoral | Imiquimod 2 | Administration |
| (single dosage | injection | mg/kg + PD-L1 | every other day with |
| form) | | monoclonal | a total of 3 times |
| | | antibody RB0005 | |
| | | 1 mg/kg | |
| G7 trial | Intratumoral | Imiquimod 6.32 | Administration |
| group(single | injection | mg/kg + PD-L1 | every other day with |
| dosage form) | | monoclonal | a total of 3 times |
| | | antibody RB0005 | |
| | | 1 mg/kg | |
| G8 trial group | Intratumoral | Imiquimod 20 | Administration |
| (single dosage | injection | mg/kg + PD-L1 | every other day with |
| form) | | monoclonal | a total of 3 times |
| | | antibody RB0005 | |
| | | 1 mg/kg | |

### 6.3 Result

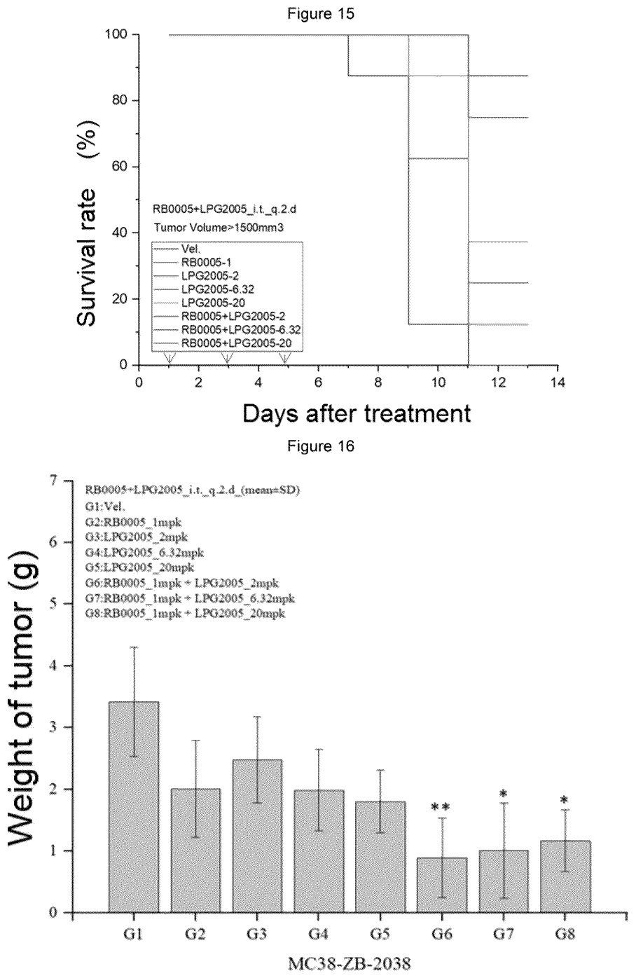

Results were shown in FIGS. 14-16 and Table 5, the RB0005 single drug had a tumor inhibition effect, and the tumor weight inhibition rate was approximately 40%; novel composite preparation groups RB0005+Imiquimod-2mpk (G6), and RB0005+Imiquimod-6.32mpk (G7) had significant inhibition effects on the tumor, the tumor weight inhibition rates were respectively 73% and 71%, which showed a synergistic drug effect, and CDI value<1, and the difference was statistically significant (p<0.05).

For the single dosage forms of the TLR7/TLR8 agonist Imiquimod and the PD-L1 monoclonal antibody, the dosage of the Imiquimod in the dosage form was 2-6.25mpk, such that the agonist had a tumor inhibition effect, and the survival rate was high (P<0.05); and the effect of the novel pharmaceutical combination was better than that of the PD-L1 monoclonal antibody RB0005 administered along, such that it indicated that the novel pharmaceutical combination had greater advantages.

TABLE 5

| Group | Tumor Weight (g) | IR_{TW} % | CDI |
|---|---|---|---|
| G1 | 3.4 | — | |
| G2 | 2.0 | 41.2 | |
| G3 | 2.5 | 27.5 | |
| G4 | 2.0 | 58.5 | |
| G5 | 1.8 | 47.4 | |
| G6 | 0.9 | 73.7 | 0.6 (<1) |
| G7 | 1.0 | 70.8 | 0.8 (<1) |
| G8 | 1.2 | 64.9 | >1 |

### Example 7

### Anti-Tumor Effect of Composite Preparation (Single Dosage Form and Separate Dosage Form) Consisting of PD-L1 Inhibitor RB0005 and TLR7/8 Agonist Imiquimod

### 7.1 Xenograft Tumor Model in Mice

After a colorectal cancer MC38 humanized CD274 cell subcutaneously-Xenograft tumor model in mice was successfully established, random grouping was performed, and each group had 6-8 mice, which were administered once every two days with a total of 3 times.

### 7.2 Grouping

| | Administration | Administration | Administration |
|---|---|---|---|
| Group | method | dosage | frequency |
| G1 control | Intraperitoneal | Solvent | Administration |
| | injection | | every other day with |
| | | | a total of 3 times |
| G2 control | Intratumoral | | |
| | injection | | |
| G3 single | Intraperitoneal | PD-L1 | Administration |
| drug group | injection | monoclonal antibody | every other day with |
| | | RB0005, 1 mg/kg | a total of 3 times |
| G4 single | Intratumoral | PD-L1 | Administration |
| drug group | injection | monoclonal antibody | every other day with |
| | | RB0005, 1 mg/kg | a total of 3 times |
| G5 single | Intraperitoneal | Imiquimod | Administration |
| drug group | injection | (LPG2005) single | every other day with |
| | | drug 5 mg/kg | a total of 3 times |
| G6 single | Intratumoral | Imiquimod | Administration |
| drug group | injection | (LPG2005) single | every other day with |
| | | drug 5 mg/kg | a total of 3 times |
| G7 trial | Intraperitoneal | (LPG2006) PD-L1 | Administration |
| group (single | injection | monoclonal | every other day with |
| preparation) | | antibody RB0005 1 | a total of 3 times |
| | | mg/kg + Imiquimod | |
| | | single drug 5 | |
| | | mg/kg | |
| G8 trial | Intratumoral | (LPG2006) PD-L1 | Administration |
| group (single | injection | monoclonal | every other day with |
| preparation) | | antibody RB0005 1 | a total of 3 times |
| | | mg/kg + Imiquimod | |
| | | single drug 5 | |
| | | mg/kg | |

### 7.3 Result

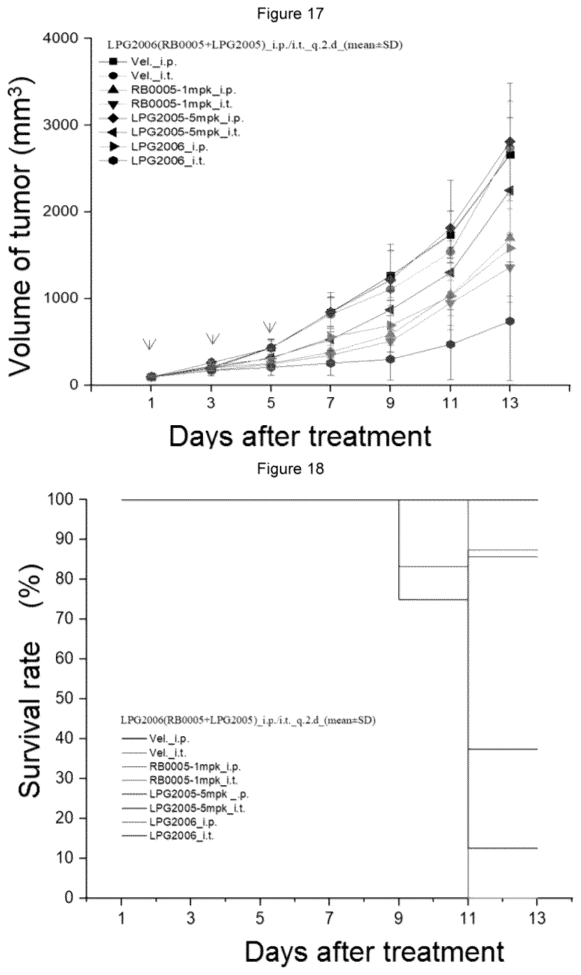

Results were shown in FIGS. 17-19 and Table 6, the tumor weight inhibition rates of the RB0005 single drug through intraperitoneal and intratumoral (G3/G4) administration were 43% and 56%; the Imiquimod single drug had better tumor inhibition effects through intraperitoneal and intratumoral (G5/G6), and intratumoral administration, and compared with Imiquimod intraperitoneal administration, difference was statistically significant (p<0.05).

The novel composite preparation single dosage form (G7/G8) was given through intraperitoneal and intratumoral administration, the tumor weight inhibition rate of intratumoral administration was 77%, such that the tumor inhibition effect was better than that of intraperitoneal administration; and the survival rate of the mice at the end of the test was 100%, a synergistic drug effect was shown, and the CDI value was 0.7.

For the single dosage forms of the Imiquimod and the PD-L1 monoclonal antibody, intratumoral administration (local) had the better tumor inhibition effect, and the survival rate was high (P<0.05), which was better than that of the composite preparation through intraperitoneal administration and each singly drug administered alone, such that it indicated that the novel pharmaceutical combination had greater advantages.

TABLE 6

| Group | Tumor Weight (g) | IR_{TW} % | CDI |
|---|---|---|---|
| G1 | 3.17 | — | |
| G2 | 3.4 | — | |
| G3 | 1.8 | 43.2 | |
| G4 | 1.5 | 55.9 | |
| G5 | 3.74 | — | |
| G6 | 2.6 | 23.5 | |
| G7 | 2.16 | 31.9 | >1 |
| G8 | 0.78 | 77.1 | 0.7 (<1) |

### Example 8

### Anti-Tumor Effect of Pharmaceutical Combination Consisting of PD-L1 Inhibitor RB0005 and Other Types of TLR7/8 Agonists (Gardiquimod)

### 8.1 Xenograft Tumor Model in Mice

After a colorectal cancer MC38 humanized CD274 cell subcutaneously-Xenograft tumor model in mice was successfully established, random grouping was performed, and each group had 6-8 mice, which were administered once every two days with a total of 3 times.

### 8.2 Grouping

| | Administration | Administration | Administration |
|---|---|---|---|
| Group | method | dosage | frequency |
| G1 control | Intratumoral | Solvent | Administration |
| | injection | | every other |
| | | | day with |
| | | | a total of 3 times |
| G2 single drug | Intratumoral | PD-L1 monoclonal | Administration |
| group | injection | antibody | every other |
| | | RB0005 single | day with |
| | | drug, 1 mg/kg | a total of 3 times |
| G3 single drug | Intratumoral | Gardiquimod | Administration |
| group | injection | single drug 5 mg/kg | every other |
| | | | day with |
| | | | a total of 3 times |
| G4 trial group | Intratumoral | PD-L1 monoclonal | Administration |
| (single | injection | antibody | every other |
| preparation) | | RB0005 1 mg/kg + | day with |
| | | Gardiquimod | a total of 3 times |
| | | single drug 5 mg/kg | |

### 8.3 Result

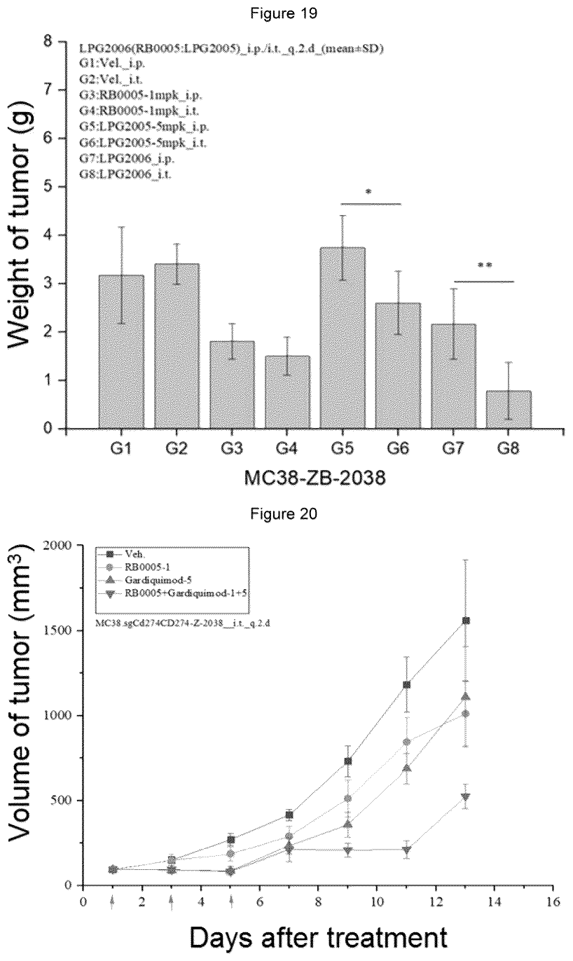

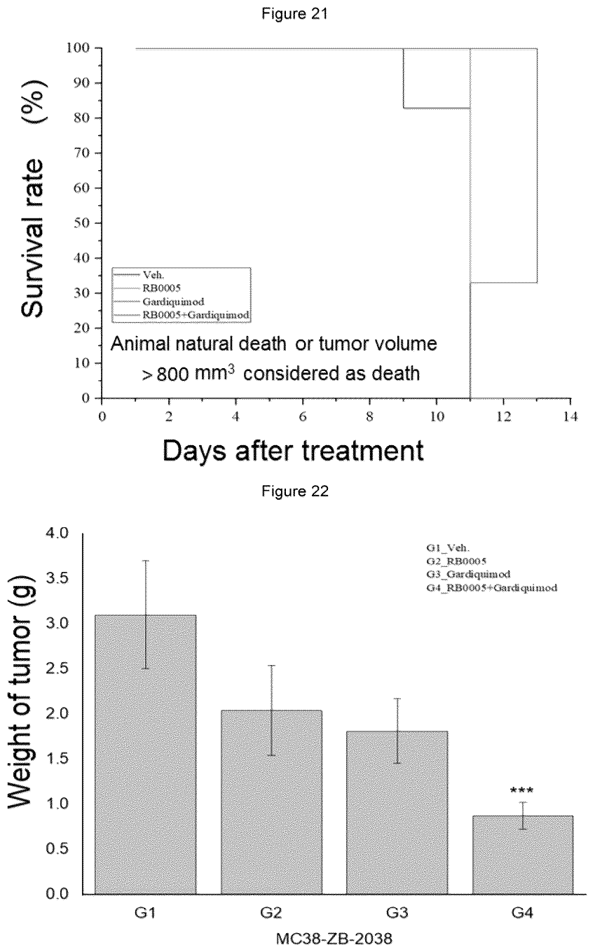

Results were shown in FIGS. 20-22 and Table 7, the tumor weight inhibition rate of the RB0005 single drug was 34%, the tumor weight inhibition rate of the Gardiquimod single drug was 42%, the tumor weight inhibition rate of the composite preparation single dosage form (G4) was 72%, and the survival rate of the mice administered with the composite preparation was 100%.

The composite preparation single dosage form formed with the Gardiquimod through intratumoral administration showed the synergistic drug effect, with CDI value<1.

The single dosage forms of the TLR7/TLR8 agonist Gardiquimod and the PD-L1 monoclonal antibody had the tumor inhibition effect showed the synergistic drug effect, the survival rate in the test was high (P<0.05), and the effect of the combination was better than that administered along, such that it indicated that the novel pharmaceutical combination had greater advantages.

TABLE 7

| Group | Tumor Weight (g) | IR_{TW} % | CDI |
|---|---|---|---|
| G1 | 3.10 | — | |
| G2 | 2.04 | 34.1 | |
| G3 | 1.81 | 41.6 | |
| G4 | 0.87 | 71.9 | 0.7 (<1) |

### Example 9

### Anti-Tumor Effect of Pharmaceutical Combination Consisting of PD-L1 Inhibitor RB0005 and Other Types of TLR7/8 Agonists (Resiquimod)

### 9.1 Xenograft Tumor Model in Mice

After a colorectal cancer MC38 humanized CD274 cell subcutaneously-Xenograft tumor model in mice was successfully established, random grouping was performed, and each group had 6-8 mice, which were administered once every two days with a total of 3 times.

### 9.2 Grouping

| | Administration | Administration | Administration |
|---|---|---|---|
| Group | method | dosage | frequency |
| G1 control | Intratumoral | Solvent | Administration every |
| | injection | | other day with |
| | | | a total of 3 times |
| G2 single | Intratumoral | PD-L1 monoclonal | Administration every |
| drug group | injection | antibody | other day with |
| | | RB0005 single | a total of 3 times |
| | | drug, 1 mg/kg | |
| G3 single | Intratumoral | Resiquimod single | Administration every |
| drug group | injection | drug 5 mg/kg | other day with |
| | | | a total of 3 times |
| G4 trial | Intratumoral | PD-L1 monoclonal | Administration every |
| group(single | injection | antibody | other day with |
| preparation) | | RB0005 1 mg/kg + | a total of 3 times |
| | | Resiquimod | |
| | | single drug 5 mg/kg | |

### 9.3 Result

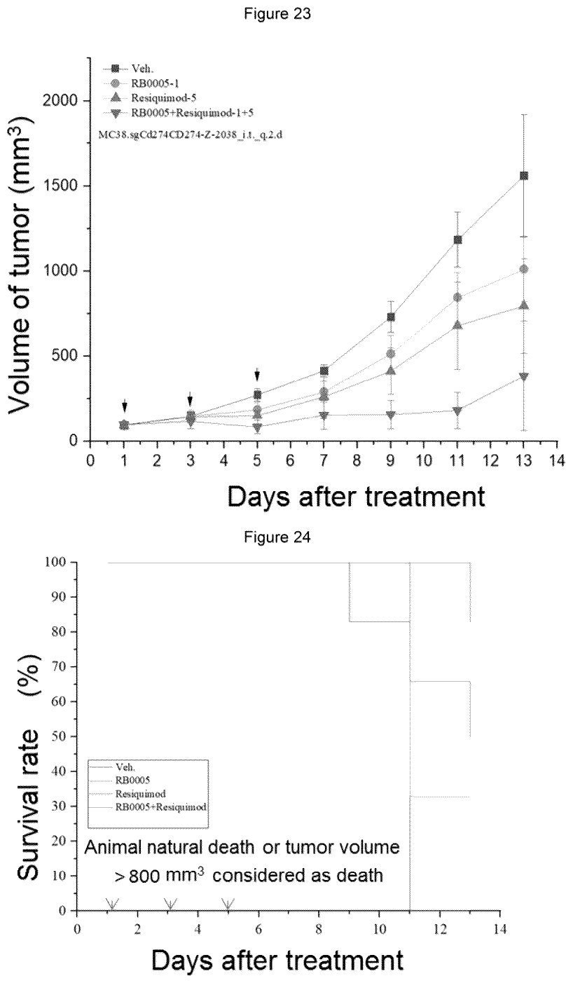

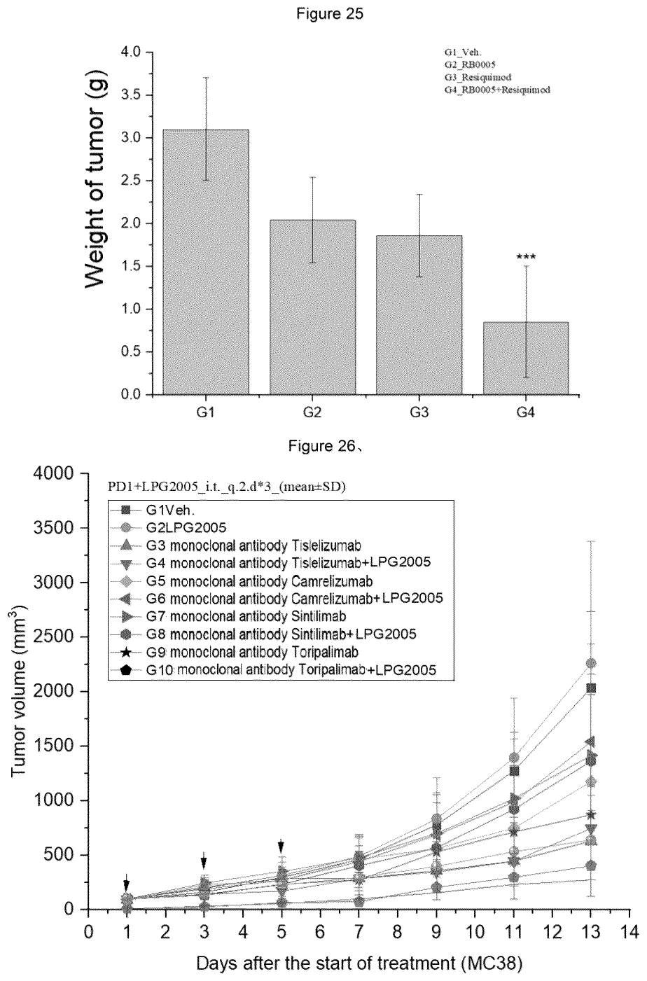

Results were shown in FIGS. 23-25 and Table 8, the tumor weight inhibition rate of the Resiquimod single drug was 40%, the tumor weight inhibition rate of the composite preparation single dosage form (G4) was 73%, the survival rate of the mice administered with the composite preparation was 85%, and the synergistic drug effect was shown, with CDI value<1.

The single dosage forms of the TLR7/TLR8 agonist Resiquimod and the PD-L1 monoclonal antibody had the tumor inhibition effect showed the synergistic drug effect, the survival rate in the test was high (P<0.05), and the effect of the combination was better than that administered along.

TABLE 8

| Group | Tumor Weight (g) | IR_{TW} % | CDI |
|---|---|---|---|
| G1 | 3.10 | — | |
| G2 | 2.04 | 34.1 | |
| G3 | 1.81 | 40.2 | |
| G4 | 0.85 | 72.6 | 0.7 (<1) |

### Example 10

### 10.1 Anti-Tumor Effect of Composite Preparation (Single Dosage Form) Consisting of a Marketed PD-1 or PD-L1 Inhibitor and a TLR7/8 Agonist Imiquimod

### 10.1.1 Xenograft Tumor Model in Mice

After a colorectal cancer MC38 humanized PD1 subcutaneous xenograft tumor model was successfully established, random grouping was performed, and each group had 6-8 mice, which were administered once every two days with a total of 3 times.

### 10.1.2 Grouping

| | Administration | Administration | Administration |
|---|---|---|---|
| Group | method | dosage | frequency |
| G1 control | Intratumoral | — | Administration |
| group | injection | | every other |
| | | | day with a |
| | | | total of 3 times |
| G2 single drug | Intratumoral | (LPG2005) Imiquimod, | Administration |
| group | injection | 5 mg/kg | every other |
| | | | day with a |
| | | | total of 3 times |
| G3 single drug | Intratumoral | Tislelizumab | Administration |
| group | injection | monoclonal antibody, | every other |
| | | 10 mg/kg | day with a |
| | | | total of 3 times |
| G4 trial group | Intratumoral | Tislelizumab | Administration |
| (single | injection | monoclonal antibody | every other |
| preparation) | | 10 mg/kg + LPG2005 | day with a |
| | | single drug 5 | total of 3 times |
| | | mg/kg | |
| G5 single drug | Intratumoral | Camrelizumab | Administration |
| group | injection | monoclonal | every other |
| | | antibody, 10 mg/kg | day with a |
| | | | total of 3 times |
| G6 trial group | Intratumoral | Camrelizumab | Administration |
| (single | injection | monoclonal antibody | every other |
| preparation) | | 10 mg/kg + LPG2005 | day with a |
| | | single drug 5 | total of 3 times |
| | | mg/kg | |
| G7 single drug | Intratumoral | Sintilimab monoclonal | Administration |
| group | injection | antibody, 10 | every other |
| | | mg/kg | day with a |
| | | | total of 3 times |
| G8 trial group | Intratumoral | Sintilimab monoclonal | Administration |
| (single | injection | antibody 10 | every other |
| preparation) | | mg/kg + LPG2005 | day with a |
| | | single drug 5 | total of 3 times |
| | | mg/kg | |
| G9 single drug | Intratumoral | Toripalimab | Administration |
| group | injection | monoclonal antibody, | every other |
| | | 10 mg/kg | day with a |
| | | | total of 3 times |
| G10 trial | Intratumoral | Toripalimab | Administration |
| group (single | injection | monoclonal antibody | every other |
| preparation) | | 10 mg/kg + LPG2005 | day with a |
| | | single drug 5 | total of 3 times |
| | | mg/kg | |

### 10.1.3 Result

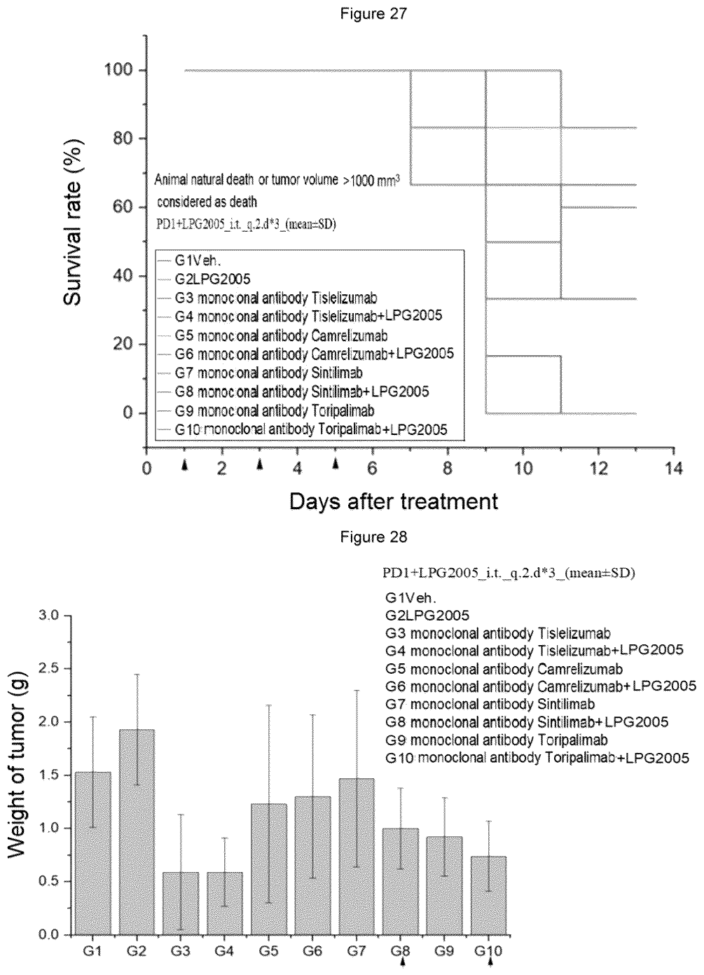

Results were shown in FIGS. 26-28 and Table 9, for the intratumoral administration of the marketed PD-1 inhibitor single drug (G3/G5/G7/G9), the G3 (Tislelizumab) had a better tumor inhibition effect, and the tumor weight inhibition rate was 61.4%.

For the novel composite preparation single dosage form (G4/G6/G8/G10), the tumor weight inhibition rates of the G8 (Sintilimab) and the G10 (Toripalimab) through intratumoral administration were 35% and 60%, the synergistic drug effect (CDI value<1) was shown, and the survival rate of the mice at the end of the test was above 60%.

In the marketed PD-1 inhibitor single drug, the intratumoral administration of the Tislelizumab monoclonal antibody had better tumor inhibition effect; and in the composite preparation formed with the TLR7/8 agonist Imiquimod, the composite preparations respectively containing the Sintilimab monoclonal antibody and the Toripalimab monoclonal antibody had the tumor inhibition effect, and had high survival rates (P<0.05), such that the pharmaceutical combination had greater advantages.

TABLE 9

| Group | Tumor Weight (g) | IR_{TW} % | CDI |
|---|---|---|---|
| G1 | 1.53 | — | |
| G2 | 1.93 | — | |
| G3 | 0.59 | 61.4 | |
| G4 | 0.59 | 61.4 | |
| G5 | 1.23 | 19.6 | |
| G6 | 1.3 | 15 | |
| G7 | 1.47 | 3.9 | |
| G8 | 1 | 34.6 | 0.54 |
| G9 | 0.92 | 49.7 | |
| G10 | 0.74 | 59.6 | 0.77 |

### 10.2 Anti-Tumor Effect of Composite Preparation (Single Dosage Form) Consisting of a Marketed PD-L1 Inhibitor and a TLR7/8 Agonist Imiquimod

### 10.2.1 Xenograft Tumor Model in Mice

After a colorectal cancer MC38 humanized CD274 cell subcutaneously-Xenograft tumor model in mice was successfully established, random grouping was performed, and each group had 6-8 mice, which were administered once every two days with a total of 3 times.

### 10.2.2 Grouping

| | Administration | Administration | Administration |
|---|---|---|---|
| Group | method | dosage | frequency |
| G1 control | Intratumoral | — | Administration |
| group | injection | | every other |
| | | | day with a |
| | | | total of 3 times |
| G2 single drug | Intratumoral | (LPG2005) | Administration |
| group | injection | Imiquimod, 5 mg/kg | every other |
| | | | day with a |
| | | | total of 3 times |
| G3 single drug | Intratumoral | Durvalumab | Administration |
| group | injection | monoclonal | every other |
| | | antibody, 1 mg/kg | day with a |
| | | | total of 3 times |
| G4 trial group | Intratumoral | Durvalumab 1 | Administration |
| (single | injection | mg/kg + LPG2005 | every other |
| preparation) | | single drug 5 mg/kg | day with a |
| | | | total of 3 times |
| G5 single drug | Intratumoral | RB0005, 1 mg/kg | Administration |
| group | injection | | every other |
| | | | day with a |
| | | | total of 3 times |
| G6 trial group | Intratumoral | RB0005 1 | Administration |
| (single | injection | mg/kg + LPG2005 | every other |
| preparation) | | single drug 5 mg/kg | day with a |
| | | | total of 3 times |

### 10.2.3 Result

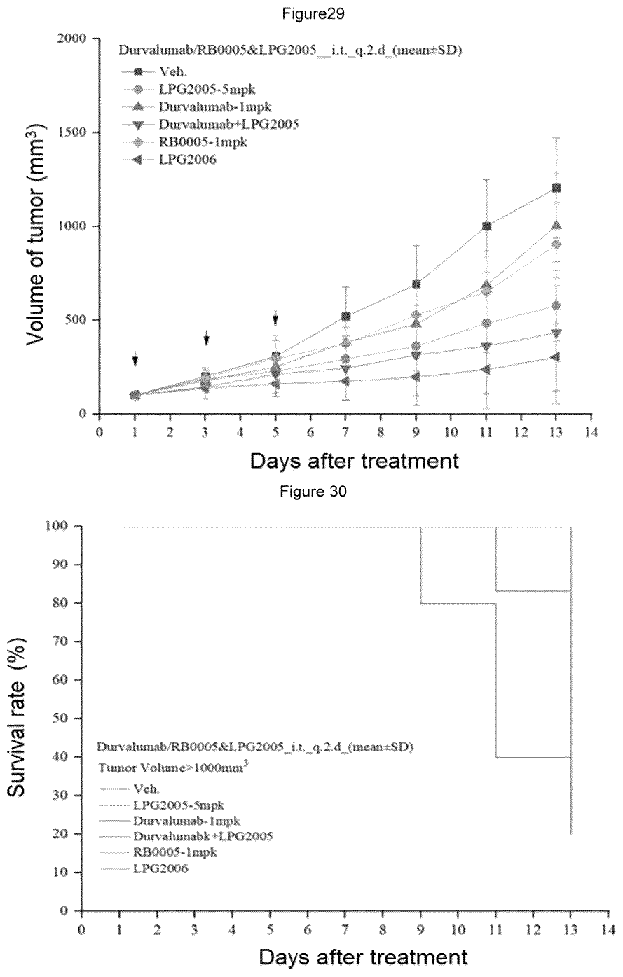

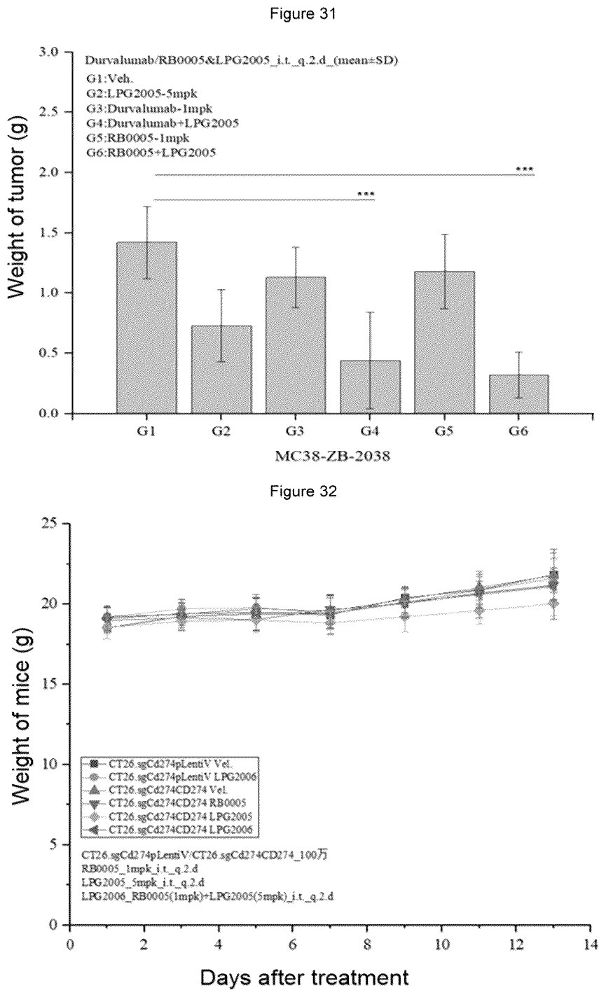

Results were shown in FIGS. 29-31 and Table 10, the tumor weight inhibition rate of the marketed PD-1 inhibitor Durvalumab single drug (G3) through intratumoral injection was 21%, and the novel composite preparation (G4) formed with the TLR7/8 agonist Imiquimod was 69%, such that the synergistic drug effect was shown, and the survival rate of the composite preparation was above 80%.

The tumor weight inhibition rate of the novel composite preparation (G6) consisting of the RB0005 and the TLR7/8 agonist Imiquimod was 78%, the synergistic drug effect (CDI value≤0.5) was shown, and the survival rate of the composite preparation at the end of the test was 100%.

In the novel composite preparation single dosage form, the composite preparation containing the RB0005 had a more significant tumor inhibition effect.

The composite preparations respectively consisting of the marketed PD-1 inhibitor Durvalumab and the RB0005 and the TLR7/8 agonist Imiquimod both showed the tumor inhibition effect and synergistic effects of the two drug components contained therein, and the survival rate was high (P<0.05), such that it indicated that the pharmaceutical combination more anti-tumor benefits than the single drug, especially the pharmaceutical combination of the RB0005.

TABLE 10

| Group | Tumor Weight (g) | IR_{TW} % | CDI |
|---|---|---|---|
| G1 | 1.42 | — | |
| G2 | 0.73 | 48.8 | |
| G3 | 1.13 | 20.9 | |
| G4 | 0.44 | 69.1 | 0.8 (<1) |
| G5 | 1.18 | 17.1 | |
| G6 | 0.32 | 77.5 | 0.5 (<1) |

### Example 11

### Combination LPG2006 (Single Dosage Form) Consisting of PD-L1 Inhibitor RB0005 and Imiquimod, CT26 Tumor Model

### 11.1 Xenograft Tumor Model in Mice

After a colorectal cancer CT26 humanized CD274 cell subcutaneous xenograft tumor model in mice was successfully established, random grouping was performed, and each group had 6-8 mice, which were administered once every two days with a total of 3 times.

### 11.2 Grouping

| | Administration | Administration | Administration |
|---|---|---|---|
| Group | method | dosage | frequency |
| G1 control group | Intratumoral | — | Administration |
| CT26.sgcd274 | injection | | every other |
| PlentiV | | | day with a |
| | | | total of 3 times |
| G2 trial group | Intratumoral | (LPG2006) | Administration |
| (single preparation) | injection | RB0005 1 mg/kg + | every other |
| CT26.sgcd274 | | LPG2005 single | day with a |
| CD274 | | drug 5 mg/kg | total of 3 times |
| G3 single drug group | Intratumoral | — | Administration |
| CT26.sgcd274 | injection | | every other |
| CD274 | | | day with a |
| | | | total of 3 times |
| G4 single drug group | Intratumoral | RB0005, 1 mg/kg | Administration |
| CT26.sgcd274 | injection | | every other |
| CD274 | | | day with a |
| | | | total of 3 times |
| G5 single drug group | Intratumoral | (LPG2005) | Administration |
| CT26.sgcd274 | injection | Imiquimod, 5 | every other |
| CD274 | | mg/kg | day with a |
| | | | total of 3 times |
| G6 trial group | Intratumoral | (LPG2006) | Administration |
| (single preparation) | injection | RB0005 1 mg/kg + | every other |
| CT26.sgcd274 | | LPG2005 single | day with a |
| CD274 | | drug 5 mg/kg | total of 3 times |

### 11.3 Result

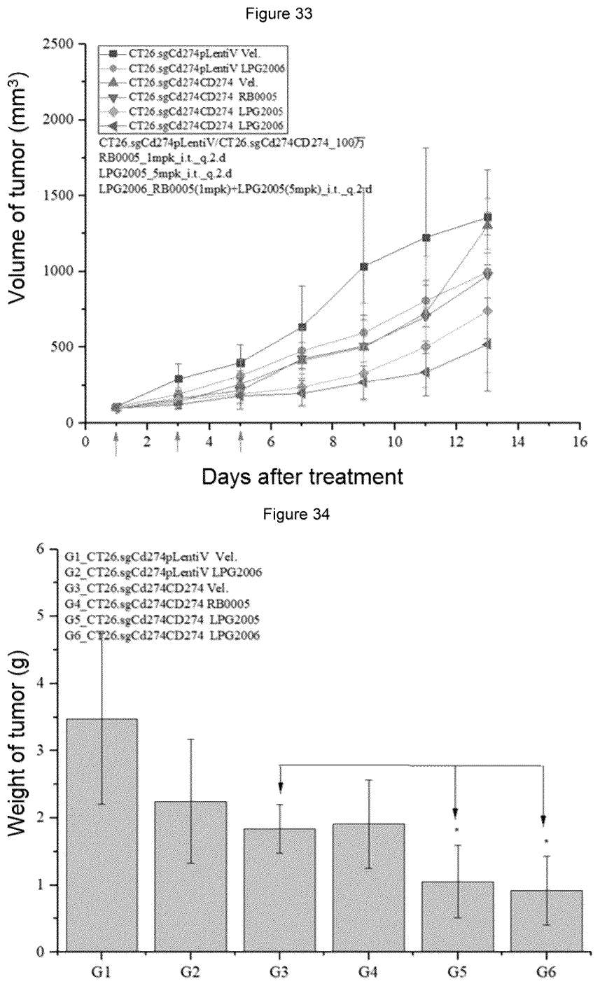

Results were shown in FIGS. 32-34 and Table 11, the RB0005 single drug showed no tumor inhibition effect in the CT26 model, but the tumor weight inhibition rate of the composite preparation single dosage form (G6) was 51%, and the synergistic drug effect was shown, with CDI value<1.

The CT26 model was not sensitive to the RB0005 single drug, but the single dosage form consisting of the RB0005 and the TLR7/TLR8 agonist Imiquimod had the tumor inhibition effect and showed the synergistic drug effect, such that it indicated that the effect of the combination was better than that of individual administration, and the sensitivity of the tumor model to the PD-L1 inhibitor was improved.

TABLE 11

| Group | Tumor Weight (g) | IR_{TW} % | CDI |
|---|---|---|---|
| G3 | 1.84 | | |
| G4 | 1.91 | — | |
| G5 | 1.05 | 42 | |
| G6 | 0.92 | 51 | 0.8 (<1) |

### Example 12

### Inhibition of MC38 Tumor Proliferation by Sequential Administration of Imiquimod and PD-L1 Monoclonal Antibody

### 12.1 Xenograft Tumor Model in Mice

After a colorectal cancer MC38 humanized CD274 cell subcutaneous xenograft tumor model in mice was successfully established, random grouping was performed, and each group had 6-8 mice, which were administered once every two days with a total of 3 times.

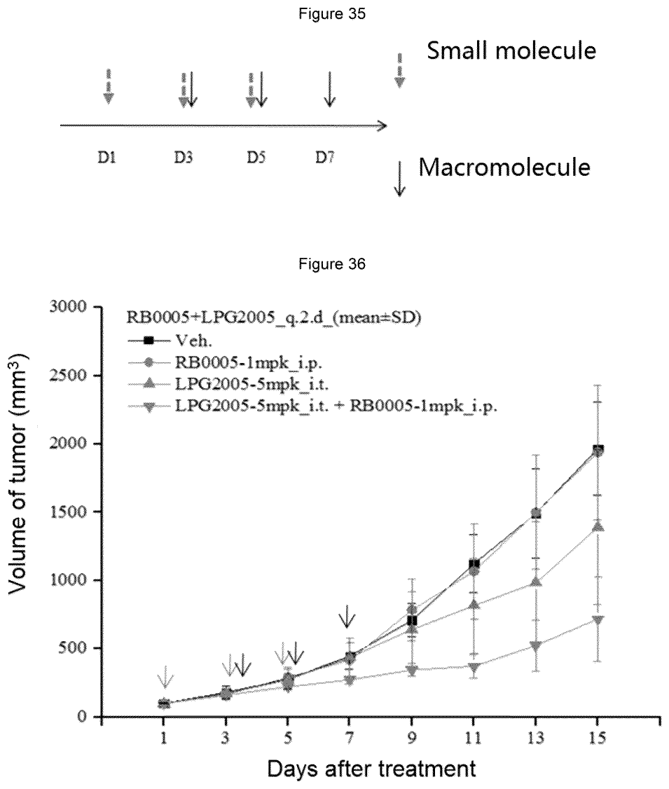

### 12.2 Grouping

| | Administration | Administration | Administration |
|---|---|---|---|
| Group | method | dosage | frequency |
| G1 control | Intratumoral | Solvent | Sequential |
| | injection | | administration, |
| | | | first administer a |
| | | | small molecule |
| G2 single | Intratumoral | PD-L1 monoclonal | agonist, and then |
| drug group | injection | antibody RB0005 | administer the |
| | | single drug, 1 mg/kg | PD-L1 inhibitor |
| G3 single | Intratumoral | (LPG2005) Imiquimod | after 48 h with a |
| drug group | injection | single drug 5 mg/kg | total of 3 |
| G4 trial | Intratumoral | PD-L1 monoclonal | times (FIG. 35) |
| group (single | injection | antibody RB0005 1 | |
| preparation) | | mg/kg + LPG2005 | |
| | | single drug 5 mg/kg | |

### 12.3 Result

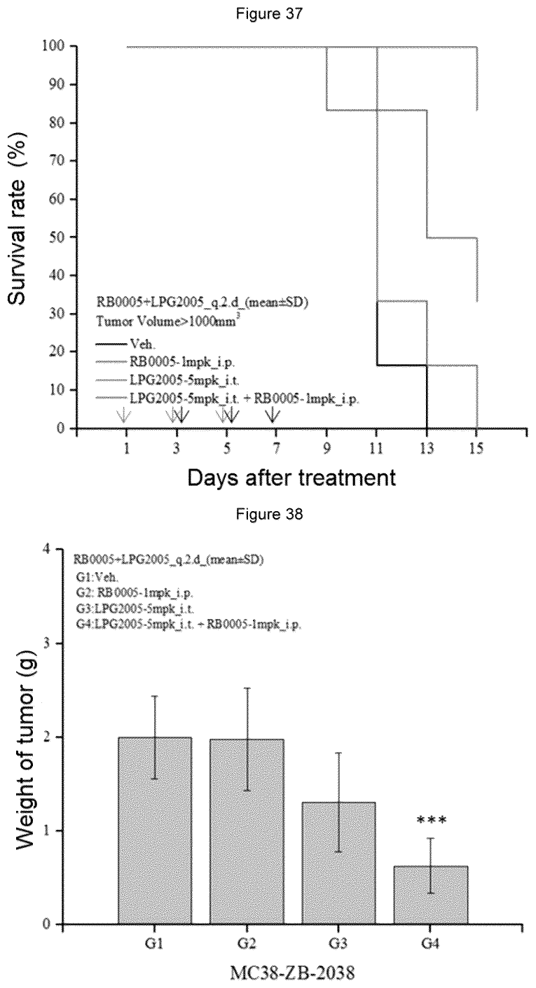

Results were shown in FIGS. 36-38 and Table 12, a sequential administration group (G4) had obvious inhibition on tumor proliferation, and after administration and within a period under observation, the proliferation of the tumor volume was slowed down. Compared the sequential administration group (G4) with the RB0005 single drug group, difference was statistically significant (ANOVA, P<0.001), and the tumor weight inhibition rate was 68%.

Sequential combination (first for small molecules, then for large molecules), the tumor inhibition effect was significant, and the synergistic drug effect was shown, with CDI=0.49. At the end of the experiment, the survival rate of the mice in a sequential combination administration group was above 80%; and compared with the control group and the RB0005 single drug group, the survival rate of the mice was significantly improved.

The Imiquimod and the PD-L1 monoclonal antibody were administered intratumorally by means of giving the small molecules followed by the PD-L1 inhibitor for 48 h, the tumor inhibition effect was significant, the synergistic drug effect was shown, and the survival rate (P<0.05) of the mice was improved, such that sequential administration had significant advantages over the single drug.

TABLE 12

| Group | Tumor Weight (g) | IR_{TW} % | CDI |
|---|---|---|---|
| G1 | 2.00 | — | |
| G2 | 1.98 | 8.4 | |
| G3 | 1.30 | 34.8 | |
| G4 | 0.63 | 68.4 | 0.49 (<1) |

What is claimed is:

1. A pharmaceutical combination comprising a programmed cell death protein 1 (PD-1) inhibitor and/or a programmed death ligand 1 (PD-L1) inhibitor, and a Toll Like Receptor (TLR) agonist.

2. The pharmaceutical combination according to claim 1, wherein a TLR comprises a TLR1, a TLR2, a TLR3, a TLR4, a TLR5, a TLR6, a TLR7, a TLR8, a TLR9, and/or a TLR10;
preferably, the TLR agonist is selected from one or more of a TLR7 agonist, a TLR8 agonist, or a TLR9 agonist;
more preferably, the TLR agonist comprises a TLR7 and TLR8 dual agonist (TLR7/TLR8 agonist);
or the TLR agonist comprises dsRNA, ssRNA, CpG DNA, an imidazole quinoline derivative, and/or a guanosine analogue;
or the TLR agonist comprises the imidazole quinoline derivative;
preferably, the TLR agonist is selected from one or more of Imiquimod, Gardiquimod, Resiquimod, 1V/209, Selgantolimod (GS-9688), Vesatolimod (GS-9620), Sumanirole, PF-4878691, or pharmaceutically acceptable derivatives thereof;
or the TLR agonist comprises the Imiquimod, Resiquimod, or pharmaceutically acceptable salts thereof:
preferably, the TLR agonist is selected from one or more of LHC-165, NKTR-262, DN1508052-01, SHR2150, CL307, CL264, Loxoribine, Isatoribine, DSR-6434,GSK2245035, SM-276001, SM-324405, SM-324406, AZ12441970, AZ12443988, or pharmaceutically acceptable derivatives thereof.

3. (canceled)

4. (canceled)

5. (canceled)

6. (canceled)

7. (canceled)

8. (canceled)

9. (canceled)

10. The pharmaceutical combination according to claim 1, wherein the PD-1 inhibitor has one or more of the following characteristics: a. inhibition or reduction of PD-L1 expression, such as transcription or translation of PD-L1; b. inhibition or reduction of PD-1 activity, such as inhibition or reduction of PD-1 binding to its homologous ligands, such as PD-L1 or PD-L2; and c. binding PD-1 or one or more of its ligands, such as PD-L1 or PD-L2;
the PD-1 inhibitor comprises an anti PD-1 antibody or antigen-binding fragments thereof;
preferably, the anti-PD-1 antibody is selected from Pembrolizumab, Nivolumab, Pidilizumab, Tislelizumab, Camrelizumab (SHR-1210), Sintilimab, Toripalimab, MEDI0680, BGB-A317, TSR-042, REGN2810, PF-06801591, RB0004, analogues thereof, or a combination thereof;
more preferably, the anti PD-1 antibody comprises at least one CDR in the antibody heavy chain variable region (VH), and the VH comprises an amino acid sequence shown in SEQ ID NO: 8;
more preferably, the anti PD-1 antibody comprises VH comprising HCDR3, and the HCDR3 comprises an amino acid sequence shown in SEQ ID NO: 3;
more preferably, the VH further comprises HCDR2, wherein the HCDR2 comprises an amino acid sequence shown in SEQ ID NO: 2;
more preferably, the VH further comprises HCDR1, wherein the HCDR1 comprises an amino acid sequence shown in SEQ ID NO: 1;
further preferably, the VH comprises HCDR1, HCDR2, and HCDR3, wherein the HCDR3 comprises an amino acid sequence shown in SEQ ID NO: 3, the HCDR2 comprises an amino acid sequence shown in SEQ ID NO: 2, and the HCDR1 comprises an amino acid sequence shown in SEQ ID NO: 1.

11. (canceled)

12. (canceled)

13. (canceled)

14. (canceled)

15. (canceled)

16. (canceled)

17. (canceled)

18. The pharmaceutical combination according to claim 10, wherein the VH comprises a framework region HFR1, the C-terminal of HFR1 is directly or indirectly connected to the N-terminal of HCDR1, and the HFR1 comprises an amino acid sequence shown in SEQ ID NO: 4 or an amino acid sequence having at least about 70% sequence identity to the amino acid sequence shown in SEQ ID NO: 4;
preferably, the VH comprises a framework region HFR2, the N-terminal of HFR2 is directly or indirectly connected to the C-terminal of HCDR1, and the C-terminal of HFR2 is directly or indirectly connected to the N-terminal of HCDR2; and the HFR2 comprises an amino acid sequence shown in SEQ ID NO: 5 or an amino acid sequence having at least about 70% sequence identity to the amino acid sequence shown in SEQ ID NO: 5;
preferably, the VH comprises a framework region HFR3, the N-terminal of HFR3 is directly or indirectly connected to the C-terminal of HCDR2, and the C-terminal of HFR3 is directly or indirectly connected to the N-terminal of HCDR3; and the HFR3 comprises an amino acid sequence shown in SEQ ID NO: 6 or an amino acid sequence having at least about 70% sequence identity to the amino acid sequence shown in SEQ ID NO: 6;
preferably, the VH comprises a framework region HFR4, the N-terminal of HFR4 is directly or indirectly connected to the C-terminal of HCDR3, and the HFR4 comprises an amino acid sequence shown in SEQ ID NO: 7 or an amino acid sequence having at least about 70% sequence identity to the amino acid sequence shown in SEQ ID NO: 7;
more preferably, the VH comprises framework regions HFR1, HFR2, HFR3, and HFR4, the C-terminal of HFR1 is directly or indirectly connected to the N-terminal of HCDR1, the N-terminal of HFR2 is directly or indirectly connected to the C-terminal of HCDR1, and the C-terminal of HFR2 is directly or indirectly connected to the N-terminal of HCDR2, the N-terminal of HFR3 is directly or indirectly connected to the C-terminal of HCDR2, and the C-terminal of HFR3 is directly or indirectly connected to the N-terminal of HCDR3, the N-terminal of HFR4 is directly or indirectly connected to the C-terminal of HCDR3; among them, the HFR1 comprises an amino acid sequence shown in SEQ ID NO: 4 or an amino acid sequence having at least about 70% sequence identity to the amino acid sequence shown in SEQ ID NO: 4, the HFR2 comprises an amino acid sequence shown in SEQ ID NO: 5 or an amino acid sequence having at least about 70% sequence identity to the amino acid sequence shown in SEQ ID NO: 5, the HFR3 comprises an amino acid sequence shown in SEQ ID NO: 6 or an amino acid sequence having at least about 70% sequence identity to the amino acid sequence shown in SEQ ID NO: 6, and the HFR4 comprises an amino acid sequence shown in SEQ ID NO: 7 or an amino acid sequence having at least about 70% sequence identity to the amino acid sequence shown in SEQ ID NO: 7.

19. (canceled)

20. (canceled)

21. (canceled)

22. (canceled)

23. The pharmaceutical combination according to claim 10, wherein the anti PD-1 antibody comprises VH, and the VH comprises an amino acid sequence shown in SEQ ID NO: 8;
preferably, the anti PD-1 antibody comprises antibody heavy chain (HC), and the HC comprises an amino acid sequence shown in SEQ ID NO: 9;
preferably, the anti PD-1 antibody comprises at least one CDR in the antibody light chain variable region (VL), and the VL comprises an amino acid sequence shown in SEQ ID NO: 17;
preferably, the anti PD-1 antibody comprises at least one CDR in VH, the VH comprises an amino acid sequence shown in SEQ ID NO: 8, and the anti PD-1 antibody comprises at least one CDR in VL, and the VL comprises an amino acid sequence shown in SEQ ID NO: 17.

24. (canceled)

25. (canceled)

26. (canceled)

27. The pharmaceutical combination according to claim 10, wherein the anti PD-1 antibody comprises VL comprising LCDR1, and the LCDR1 comprises an amino acid sequence shown in SEQ ID NO: 10;
preferably, the VL further comprises LCDR2, wherein the LCDR2 comprises an amino acid sequence shown in SEQ ID NO: 11;
preferably, the VL further comprises LCDR3, wherein the LCDR3 comprises an amino acid sequence shown in SEQ ID NO: 12:
preferably, the VL comprises LCDR1, LCDR2 and LCDR3, wherein the LCDR1 comprises an amino acid sequence shown in SEQ ID NO: 10, the LCDR2 comprises an amino acid sequence shown in SEQ ID NO: 11, and the LCDR3 comprises an amino acid sequence shown in SEQ ID NO: 12.

28. (canceled)

29. (canceled)

30. (canceled)

31. The pharmaceutical combination according to claim 10, wherein the anti PD-1 antibody comprises VH and antibody VL, the VH comprises HCDR1, HCDR2, and HCDR3, wherein the HCDR3 comprises an amino acid sequence shown in SEQ ID NO: 3, the HCDR2 comprises an amino acid sequence shown in SEQ ID NO: 2, and the HCDR1 comprises an amino acid sequence shown in SEQ ID NO: 1; and the VL comprises LCDR1, LCDR2, and LCDR3, wherein the LCDR1 comprises an amino acid sequence shown in SEQ ID NO: 10, the LCDR2 comprises an amino acid sequence shown in SEQ ID NO: 11, and the LCDR3 comprises an amino acid sequence shown in SEQ ID NO: 12.

32. The pharmaceutical combination according to claim 27, wherein the VL comprises a framework region LFR1, the C-terminal of LFR1 is directly or indirectly connected to the N-terminal of LCDR1, and the LFR1 comprises an amino acid sequence shown in SEQ ID NO: 13 or an amino acid sequence having at least about 70% sequence identity to the amino acid sequence shown in SEQ ID NO: 13;
preferably, the VL comprises a framework region LFR2, the N-terminal of LFR2 is directly or indirectly connected to the C-terminal of LCDR1, and the C-terminal of LFR2 is directly or indirectly connected to the N-terminal of LCDR2; and the LFR2 comprises an amino acid sequence shown in SEQ ID NO: 14 or an amino acid sequence having at least about 70% sequence identity to the amino acid sequence shown in SEQ ID NO: 14;
preferably, the VL comprises a framework region LFR3, the N-terminal of LFR3 is directly or indirectly connected to the C-terminal of LCDR2, and the C-terminal of LFR3 is directly or indirectly connected to the N-terminal of LCDR3; and the LFR3 comprises an amino acid sequence shown in SEQ ID NO: 15 or an amino acid sequence having at least about 70% sequence identity to the amino acid sequence shown in SEQ ID NO: 15:
preferably, the VL comprises a framework region LFR4, the N-terminal of LFR4 is directly or indirectly connected to the C-terminal of LCDR3, and the LFR4 comprises an amino acid sequence shown in SEQ ID NO: 16 or an amino acid sequence having at least about 70% sequence identity to the amino acid sequence shown in SEQ ID NO: 16.

33. (canceled)

34. (canceled)

35. (canceled)

36. The pharmaceutical combination according to claim 27, wherein the VL comprises framework regions LFR1, LFR2, LFR3 and LFR4, the C-terminal of LFR1 is directly or indirectly connected to the N-terminal of LCDR1, the N-terminal of LFR2 is directly or indirectly connected to the C-terminal of LCDR1, and the C-terminal of LFR2 is directly or indirectly connected to the N-terminal of LCDR2, the N-terminal of LFR3 is directly or indirectly connected to the C-terminal of LCDR2, and the C-terminal of LFR3 is directly or indirectly connected to the N-terminal of LCDR3, the N-terminal of LFR4 is directly or indirectly connected to the C-terminal of LCDR3; among them, the LFR1 comprises an amino acid sequence shown in SEQ ID NO: 13 or an amino acid sequence having at least about 70% sequence identity to the amino acid sequence shown in SEQ ID NO: 13, the LFR2 comprises an amino acid sequence shown in SEQ ID NO: 14 or an amino acid sequence having at least about 70% sequence identity to the amino acid sequence shown in SEQ ID NO: 14, the LFR3 comprises an amino acid sequence shown in SEQ ID NO: 15 or an amino acid sequence having at least about 70% sequence identity to the amino acid sequence shown in SEQ ID NO: 15, and the LFR4 comprises an amino acid sequence shown in SEQ ID NO: 16 or an amino acid sequence having at least about 70% sequence identity to the amino acid sequence shown in SEQ ID NO:16.

37. The pharmaceutical combination according to claim 27, wherein the anti PD-1 antibody comprises VL, and the VL comprises an amino acid sequence shown in SEQ ID NO: 17;
preferably, the anti PD-1 antibody comprises VH and VL, the VH comprises an amino acid sequence shown in SEQ ID NO: 8 and the VL comprises an amino acid sequence shown in SEQ ID NO: 17;
preferably, the anti PD-1 antibody comprises antibody light chain (LC), and the LC comprises an amino acid sequence shown in SEQ ID NO: 18;
preferably, the anti PD-1 antibody comprises HC and LC, the HC comprises an amino acid sequence shown in SEQ ID NO: 9 and the LC comprises an amino acid sequence shown in SEQ ID NO: 18.

38. (canceled)

39. (canceled)

40. (canceled)

41. The pharmaceutical combination according to claim 1, wherein the PD-L1 inhibitor has one or more of the following characteristics: a. inhibition or reduction of PD-L1 expression, such as transcription or translation of PD-L1; b. inhibition or reduction of PD-L1 activity, such as inhibition or reduction of PD-L1 binding to its associated receptors such as PD-1; and c. binding of PD-L1 or its receptors such as PD-1;
preferably, the PD-L1 inhibitor comprises an anti PD-L1 antibody or antigen-binding fragments thereof;
preferably, the anti-PD-L1 antibody is selected from Durvalumab, Atezolizumab, Avelumab, MDX-1105, YW243.55.S70, MDPL3280A, AMP-224, LY3300054, RB0005, analogues thereof, or a combination thereof.

42. (canceled)

43. (canceled)

44. The pharmaceutical combination according to claim 41, wherein the anti PD-L1 antibody comprises at least one CDR in VH, and the VH comprises an amino acid sequence shown in SEQ ID NO: 25;
preferably, the anti PD-L1 antibody comprises VH comprising HCDR3, and the HCDR3 comprises an amino acid sequence shown in SEQ ID NO: 21;
preferably, the VH further comprises HCDR2, wherein the HCDR2 comprises an amino acid sequence shown in SEQ ID NO: 20;
preferably, the VH further comprises HCDR1, wherein the HCDR1 comprises an amino acid sequence shown in SEQ ID NO: 19;
preferably, the VH comprises HCDR1, HCDR2, and HCDR3, wherein the HCDR3 comprises an amino acid sequence shown in SEQ ID NO: 21, the HCDR2 comprises an amino acid sequence shown in SEQ ID NO: 20, and the HCDR1 comprises an amino acid sequence shown in SEQ ID NO: 19.

45. (canceled)

46. (canceled)

47. (canceled)

48. (canceled)

49. The pharmaceutical combination according to claim 44, wherein the VH comprises a framework region HFR1, the C-terminal of HFR1 is directly or indirectly connected to the N-terminal of HCDR1, and the HFR1 comprises an amino acid sequence shown in SEQ ID NO: 22 or an amino acid sequence having at least about 70% sequence identity to the amino acid sequence shown in SEQ ID NO: 22;
preferably, the VH comprises a framework region HFR2, the N-terminal of HFR2 is directly or indirectly connected to the C-terminal of HCDR1, and the C-terminal of HFR2 is directly or indirectly connected to the N-terminal of HCDR2; and the HFR2 comprises an amino acid sequence shown in SEQ ID NO: 23 or an amino acid sequence having at least about 70% sequence identity to the amino acid sequence shown in SEQ ID NO: 23;
preferably, the VH comprises a framework region HFR3, the N-terminal of HFR3 is directly or indirectly connected to the C-terminal of HCDR2, and the C-terminal of HFR3 is directly or indirectly connected to the N-terminal of HCDR3; and the HFR3 comprises an amino acid sequence shown in SEQ ID NO: 24 or an amino acid sequence having at least about 70% sequence identity to the amino acid sequence shown in SEQ ID NO: 24;
preferably, the VH comprises a framework region HFR4, the N-terminal of HFR4 is directly or indirectly connected to the C-terminal of HCDR3, and the HFR4 comprises an amino acid sequence shown in SEQ ID NO: 7 or an amino acid sequence having at least about 70% sequence identity to the amino acid sequence shown in SEQ ID NO: 7:
more preferably, the VH comprises framework regions HFR1, HFR2, HFR3 and HFR4, the C-terminal of HFR1 is directly or indirectly connected to the N-terminal of HCDR1, the N-terminal of HFR2 is directly or indirectly connected to the C-terminal of HCDR1, and the C-terminal of HFR2 is directly or indirectly connected to the N-terminal of HCDR2, the N-terminal of HFR3 is directly or indirectly connected to the C-terminal of HCDR2, and the C-terminal of HFR3 is directly or indirectly connected to the N-terminal of HCDR3, the N-terminal of HFR4 is directly or indirectly connected to the C-terminal of HCDR3; among them, the HFR1 comprises an amino acid sequence shown in SEQ ID NO: 22 or an amino acid sequence having at least about 70% sequence identity to the amino acid sequence shown in SEQ ID NO: 22, the HFR2 comprises an amino acid sequence shown in SEQ ID NO: 23 or an amino acid sequence having at least about 70% sequence identity to the amino acid sequence shown in SEQ ID NO: 23, the HFR3 comprises an amino acid sequence shown in SEQ ID NO: 24 or an amino acid sequence having at least about 70% sequence identity to the amino acid sequence shown in SEQ ID NO: 24, and the HFR4 comprises an amino acid sequence shown in SEQ ID NO: 7 or an amino acid sequence having at least about 70% sequence identity to the amino acid sequence shown in SEQ ID NO: 7.

50. (canceled)

51. (canceled)

52. (canceled)

53. (canceled)

54. The pharmaceutical combination according to claim 41, wherein the anti PD-L1 antibody comprises VH, and the VH comprises an amino acid sequence shown in SEQ ID NO: 25;
preferably, the anti PD-L1 antibody comprises HC, and the HC comprises an amino acid sequence shown in SEQ ID NO: 26;
preferably, the anti PD-L1 antibody comprises at least one CDR in VL, and the VL comprises an amino acid sequence shown in SEQ ID NO: 37;
preferably, the anti PD-L1 antibody comprises at least one CDR in VH, the VH comprises an amino acid sequence shown in SEQ ID NO: 25, and the anti PD-L1 antibody comprises at least one CDR in VL, and the VL comprises an amino acid sequence shown in SEQ ID NO: 37;
preferably, the anti PD-L1 antibody comprises at least one CDR in VH, the VH comprises an amino acid sequence shown in SEQ ID NO: 25, and the anti PD-L1 antibody comprises at least one CDR in VL, and the VL comprises an amino acid sequence shown in SEQ ID NO:38, SEQ ID NO:39 or SEQ ID NO:40.

55. (canceled)

56. (canceled)

57. (canceled)

58. (canceled)

59. The pharmaceutical combination according to claim 41, wherein the anti PD-L1 antibody comprises VL, the VL comprises LCDR1, and the LCDR1 comprises an amino acid sequence shown in SEQ ID NO: 27;
preferably, antibody comprises VL, the VL comprises LCDR1, and the LCDR1 comprises an amino acid sequence shown in SEQ ID NO:28, SEQ ID NO:29 or SEQ ID NO: 30;
preferably, the VL further comprises LCDR2, wherein the LCDR2 comprises an amino acid sequence shown in SEQ ID NO: 31;
preferably, the VL further comprises LCDR3, wherein the LCDR3 comprises an amino acid sequence shown in SEQ ID NO: 32;
preferably, the VL comprises LCDR1, LCDR2 and LCDR3, wherein the LCDR1 comprises an amino acid sequence shown in SEQ ID NO: 27, the LCDR2 comprises an amino acid sequence shown in SEQ ID NO: 31, and the LCDR3 comprises an amino acid sequence shown in SEQ ID NO: 32.

60. (canceled)

61. (canceled)

62. (canceled)

63. (canceled)

64. The pharmaceutical combination according to claim 59, wherein the VL comprises LCDR1, LCDR2 and LCDR3, wherein the LCDR1 comprises an amino acid sequence shown in SEQ ID NO: 28, the LCDR2 comprises an amino acid sequence shown in SEQ ID NO: 31, and the LCDR3 comprises an amino acid sequence shown in SEQ ID NO: 32; the LCDR1 comprises an amino acid sequence shown in SEQ ID NO: 29, the LCDR2 comprises an amino acid sequence shown in SEQ ID NO: 31, the LCDR3 comprises an amino acid sequence shown in SEQ ID NO: 32; or the LCDR1 comprises an amino acid sequence shown in SEQ ID NO: 30, the LCDR2 comprises an amino acid sequence shown in SEQ ID NO: 31, and the LCDR3 comprises an amino acid sequence shown in SEQ ID NO: 32;
preferably, the anti PD-L1 antibody comprises VH and antibody VL, the VH comprises HCDR1, HCDR2, and HCDR3, wherein the HCDR3 comprises an amino acid sequence shown in SEQ ID NO: 21. the HCDR2 comprises an amino acid sequence shown in SEQ ID NO: 20, and the HCDR1 comprises an amino acid sequence shown in SEQ ID NO: 19; and the VL comprises LCDR1, LCDR2, and LCDR3, wherein the LCDR1 comprises an amino acid sequence shown in SEQ ID NO: 27. the LCDR2 comprises an amino acid sequence shown in SEQ ID NO: 31, and the LCDR3 comprises an amino acid sequence shown in SEQ ID NO: 32;
preferably, the anti PD-L1 antibody comprises VH and antibody VL, the VH comprises HCDR1, HCDR2, and HCDR3, wherein the HCDR3 comprises an amino acid sequence shown in SEQ ID NO: 21, the HCDR2 comprises an amino acid sequence shown in SEQ ID NO: 20, and the HCDR1 comprises an amino acid sequence shown in SEQ ID NO: 19; and the VL comprises LCDR1, LCDR2, and LCDR3, wherein the LCDR1 comprises an amino acid sequence shown in SEQ ID NO:28, SEQ ID NO:29 or SEQ ID NO:30, the LCDR2 comprises an amino acid sequence shown in SEQ ID NO: 31, and the LCDR3 comprises an amino acid sequence shown in SEQ ID NO: 32.

65. (canceled)

66. (canceled)

67. The pharmaceutical combination according to claim 59, wherein the VL comprises a framework region LFR1, the C-terminal of LFR1 is directly or indirectly connected to the N-terminal of LCDR1, and the LFR1 comprises an amino acid sequence shown in SEQ ID NO: 33 or an amino acid sequence having at least about 70% sequence identity to the amino acid sequence shown in SEQ ID NO: 33;
preferably, the VL comprises a framework region LFR2, the N-terminal of LFR2 is directly or indirectly connected to the C-terminal of LCDR1, and the C-terminal of LFR2 is directly or indirectly connected to the N-terminal of LCDR2; and the LFR2 comprises an amino acid sequence shown in SEQ ID NO: 34 or an amino acid sequence having at least about 70% sequence identity to the amino acid sequence shown in SEQ ID NO: 34;
preferably, the VL comprises a framework region LFR3, the N-terminal of LFR3 is directly or indirectly connected to the C-terminal of LCDR2, and the C-terminal of LFR3 is directly or indirectly connected to the N-terminal of LCDR3; and the LFR3 comprises an amino acid sequence shown in SEQ ID NO: 35 or an amino acid sequence having at least about 70% sequence identity to the amino acid sequence shown in SEQ ID NO: 35;
preferably, the VL comprises a framework region LFR4, the N-terminal of LFR4 is directly or indirectly connected to the C-terminal of LCDR3, and the LFR4 comprises an amino acid sequence shown in SEQ ID NO: 36 or an amino acid sequence having at least about 70% sequence identity to the amino acid sequence shown in SEQ ID NO: 36;
more preferably, the VL comprises framework regions LFR1, LFR2, LFR3 and LFR4, the C-terminal of LFR1 is directly or indirectly connected to the N-terminal of LCDR1, the N-terminal of LFR2 is directly or indirectly connected to the C-terminal of LCDR1, and the C-terminal of LFR2 is directly or indirectly connected to the N-terminal of LCDR2, the N-terminal of LFR3 is directly or indirectly connected to the C-terminal of LCDR2, and the C-terminal of LFR3 is directly or indirectly connected to the N-terminal of LCDR3, the N-terminal of LFR4 is directly or indirectly connected to the C-terminal of LCDR3; among them, the LFR1 comprises an amino acid sequence shown in SEQ ID NO: 33 or an amino acid sequence having at least about 70% sequence identity to the amino acid sequence shown in SEQ ID NO: 33, the LFR2 comprises an amino acid sequence shown in SEQ ID NO: 34 or an amino acid sequence having at least about 70% sequence identity to the amino acid sequence shown in SEQ ID NO: 34, the LFR3 comprises an amino acid sequence shown in SEQ ID NO: 35 or an amino acid sequence having at least about 70% sequence identity to the amino acid sequence shown in SEQ ID NO: 35, and the LFR4 comprises an amino acid sequence shown in SEQ ID NO: 36 or an amino acid sequence having at least about 70% sequence identity to the amino acid sequence shown in SEQ ID NO:36.

68. (canceled)

69. (canceled)

70. (canceled)

71. (canceled)

72. The pharmaceutical combination according to claim 41, wherein the anti PD-L1 antibody comprises VL, and the VL comprises an amino acid sequence shown in SEQ ID NO: 37;
preferably, the anti PD-L1 antibody comprises VL, and the VL comprises an amino acid sequence shown in SEQ ID NO:38, SEQ ID NO:39 or SEQ ID NO:40;
preferably, the anti PD-L1 antibody comprises VH and VL, the VH comprises an amino acid sequence shown in SEQ ID NO: 25, and the VL comprises an amino acid sequence shown in SEQ ID NO: 37;
preferably, the anti PD-L1 antibody comprises VH and VL, the VH comprises an amino acid sequence shown in SEQ ID NO: 25, and the VL comprises an amino acid sequence shown in SEQ ID NO:38, SEQ ID NO:39 or SEQ ID NO:40;
preferably, the anti PD-L1 antibody comprises LC, and the LC comprises an amino acid sequence shown in SEQ ID NO: 41;
preferably, the anti PD-L1 antibody comprises LC, and the LC comprises an amino acid sequence shown in SEQ ID NO:42, SEQ ID NO:43 or SEQ ID NO:44;
preferably, the anti PD-L1 antibody comprises HC and LC, the HC comprises an amino acid sequence shown in SEQ ID NO: 26, and the LC comprises an amino acid sequence shown in SEQ ID NO: 41;
preferably, the anti PD-L1 antibody comprises HC and LC, the HC comprises an amino acid sequence shown in SEQ ID NO: 26, and the LC comprises an amino acid sequence shown in SEQ ID NO:42, SEQ ID NO:43 or SEQ ID NO:44.

73. (canceled)

74. (canceled)

75. (canceled)

76. (canceled)

77. (canceled)

78. (canceled)

79. (canceled)

80. The pharmaceutical combination according to claim 1, wherein i) the PD-1 inhibitor and/or PD-L1 inhibitor in the pharmaceutical combination are not mixed with ii) the TLR agonist to each other in the pharmaceutical combination;
preferably, i) the PD-1 inhibitor and/or PD-L1 inhibitor, and ii) the TLR agonist are present in the pharmaceutical combination in a single dosage form;
preferably, the pharmaceutical combination is formulated into a pharmaceutical composition;
preferably, the pharmaceutical composition comprises a PD-1 inhibitor or a PD-L1 inhibitor, and a TLR agonist;
preferably, the TLR agonist is present in an amount of about 0.0001 mg/kg to about 200 mg/kg;
preferably, the PD-1 inhibitor or PD-L1 inhibitor is present in an amount of about 0.0001 mg/kg to about 200 mg/kg;
preferably, the pharmaceutical composition further comprises one or more pharmaceutically acceptable carriers.

81. (canceled)

82. (canceled)

83. (canceled)

84. (canceled)

85. (canceled)

86. (canceled)

87. (canceled)

88. (canceled)

89. (canceled)

90. (canceled)

91. A method for treating neoplastic diseases, comprising administering an effective amount of the pharmaceutical combination according to claim 1 to a subject in need thereof;
preferably, the subject suffers from neoplasm;
more preferably, the neoplasm comprises tumors and/or warts;
preferably, the administration comprises local, intraneoplastic or systemic administrations;
more preferably, the administration comprises intravenous injection, intravenous instillation, intramuscular injection, subcutaneous injection, and/or intraneoplastic injection;
preferably, i) the PD-1 inhibitor or PD-L1 inhibitor, and ii) the TLR agonist in the pharmaceutical combination are administered by use of the same or different administration methods;
preferably, the method comprises: injection of the TLR agonist into the neoplasm;
preferably, the method further comprises: injection or systemic infusion of the PD-1 inhibitor or PD-L1 inhibitor into the neoplasm;
preferably, injecting the i) the PD-1 inhibitor or PD-L1 inhibitor, and ii) the TLR agonist in the pharmaceutical combination into the neoplasm;
preferably, i) the PD-1 inhibitor or PD-L1 inhibitor, and ii) the TLR agonist in the pharmaceutical combination are administered simultaneously or at different times;
preferably, the PD-1 inhibitor or PD-L1 inhibitor is administered before and/or after the administration of the TLR agonist;
preferably, the method comprises: i) injecting the TLR agonist into the neoplasm; and ii) injection or systemic infusion of the PD-1 inhibitor or the PD-L1 inhibitor into the neoplasm or the whole body after the TLR agonist is administered;
preferably the method comprises: i) injection of the TLR agonist into the neoplasm; ii) injection or systemic infusion of the PD-1 inhibitor or PD-L1 inhibitor into the neoplasm after administering the TLR agonist;
preferably, the method comprises: administering the PD-1 inhibitor or the PD-L1 inhibitor about 2 h to about 72 h after administering the TLR agonis;
preferably, the method comprises: administering the PD-1 inhibitor or the PD-L1 inhibitor about 2 h, about 4 h, about 8 h, about 16 h, about 24 h, about 36 h, about 48 h, about 60 h, or about 72 h after administering the TLR agonist;
preferably, the method comprises: i) injecting the TLR agonist into the neoplasm; and ii) injecting or infusing the PD-1 inhibitor or the PD-L1 inhibitor into the neoplasm or the whole body about 48 h after the TLR agonist is administered;
preferably, the method comprises: i) the PD-1 inhibitor or PD-L1 inhibitor, and ii) the TLR agonist in the pharmaceutical combination are administered simultaneously:
more preferably, i) the PD-1 inhibitor or PD-L1 inhibitor, and ii) the TLR agonist in the pharmaceutical combination are simultaneously administered by way of intraneoplastic injection, and the PD-1 inhibitor or PD-L1 inhibitor and the TLR agonist are present in a same dosage form;
more preferably, i) the PD-1 inhibitor or the PD-L1 inhibitor in the pharmaceutical combination, and ii) the TLR agonist are administered at the same time by means of intra-neoplasm injection; and the PD-1 inhibitor or the PD-L1 inhibitor and the TLR agonist are located in separate dosage forms.

92. (canceled)

93. (canceled)

94. (canceled)

95. (canceled)

96. (canceled)

97. (canceled)

98. (canceled)

99. (canceled)

100. (canceled)

101. (canceled)

103. (canceled)

104. (canceled)

105. (canceled)

106. (canceled)

107. (canceled)

108. (canceled)

109. (canceled)

110. (canceled)

111. (canceled)


SEQUENCE LISTING (44 sequences)

SEQ ID NO: 1 - protein, 8 aa, Artificial Sequence
  RB00004 HCDR1
GFTFSSYT

SEQ ID NO: 2 - protein, 8 aa, Artificial Sequence
  RB00004 HCDR2
ISNGGSFT

SEQ ID NO: 3 - protein, 12 aa, Artificial Sequence
  RB00004 HCDR3
ARDSDYYGIFDY

SEQ ID NO: 4 - protein, 25 aa, Artificial Sequence
  RB00004 HFR1
EVQLVESGGGLVKPGGSLRLSCAAS

SEQ ID NO: 5 - protein, 17 aa, Artificial Sequence
  RB00004 HFR2
MSWVRQAPGKGLEWVAT

SEQ ID NO: 6 - protein, 38 aa, Artificial Sequence
  RB00004 HFR3
YYPDSMKGRFTISRDNSKNTLYLQMSSLRAEDTAVYYC

SEQ ID NO: 7 - protein, 11 aa, Artificial Sequence
  RB00004 HFR4RB00005 HFR4
WGQGTTVTVSS

SEQ ID NO: 8 - protein, 119 aa, Artificial Sequence
  RB00004 VH
EVQLVESGGGLVKPGGSLRLSCAASGFTFSSYTMSWVRQAPGKGLEWVATISNGGSFTYYPDSMKGRFTISRDNSKNTLYLQMSSLRAEDTAVYYCARDSDYYGIFDYWGQGTTVTVSS

SEQ ID NO: 9 - protein, 446 aa, Artificial Sequence
  RB00004 HC
EVQLVESGGGLVKPGGSLRLSCAASGFTFSSYTMSWVRQAPGKGLEWVATISNGGSFTYYPDSMKGRFTISRDNSKNTLYLQMSSLRAEDTAVYYCARDSDYYGIFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 16 aa, Artificial Sequence
  RB00004 LCDR1
RSSKSLLYKDGKTYLN

SEQ ID NO: 11 - protein, 7 aa, Artificial Sequence
  RB00004 LCDR2
LMSTRAS

SEQ ID NO: 12 - protein, 9 aa, Artificial Sequence
  RB00004 LCDR3
QQLVEDPFT

SEQ ID NO: 13 - protein, 23 aa, Artificial Sequence
  RB00004 LFR1
DIVMTQSPLSLPVTPGEPASISC

SEQ ID NO: 14 - protein, 15 aa, Artificial Sequence
  RB00004 LFR2
WYLQKPGQSPQLLIY

SEQ ID NO: 15 - protein, 32 aa, Artificial Sequence
  RB00004 LFR3
GVPDRFSGSGSGTDFTLKISRVEAEDVGVYYC

SEQ ID NO: 16 - protein, 13 aa, Artificial Sequence
  RB00004 LFR4
FGQGTKLEIKRTV

SEQ ID NO: 17 - protein, 115 aa, Artificial Sequence
  RB00004 VL
DIVMTQSPLSLPVTPGEPASISCRSSKSLLYKDGKTYLNWYLQKPGQSPQLLIYLMSTRASGVPDRFSGSGSGTDFTLKISRVEAEDVGVYYCQQLVEDPFTFGQGTKLEIKRTV

SEQ ID NO: 18 - protein, 230 aa, Artificial Sequence
  RB00004 LC
IALSLALVTNSDIVMTQSPLSLPVTPGEPASISCRSSKSLLYKDGKTYLNWYLQKPGQSPQLLIYLMSTRASGVPDRFSGSGSGTDFTLKISRVEAEDVGVYYCQQLVEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 5 aa, Artificial Sequence
  RB00005 HCDR1
KYIIH

SEQ ID NO: 20 - protein, 17 aa, Artificial Sequence
  RB00005 HCDR2
WFYPGSGNIRYNEKIKG

SEQ ID NO: 21 - protein, 12 aa, Artificial Sequence
  RB00005 HCDR3
HGELGGGYFFDY

SEQ ID NO: 22 - protein, 30 aa, Artificial Sequence
  RB00005 HFR1
EVQLVQSGAEVKKPGASVKVSCKASGYTFT

SEQ ID NO: 23 - protein, 14 aa, Artificial Sequence
  RB00005 HFR2
WVRQAPGQGLEWMG

SEQ ID NO: 24 - protein, 32 aa, Artificial Sequence
  RB00005 HFR3
RVTMTRDTSTSTVYMELSSLRSEDTAVYYCAR

SEQ ID NO: 25 - protein, 121 aa, Artificial Sequence
  RB00005 VH
EVQLVQSGAEVKKPGASVKVSCKASGYTFTKYIIHWVRQAPGQGLEWMGWFYPGSGNIRYNEKIKGRVTMTRDTSTSTVYMELSSLRSEDTAVYYCARHGELGGGYFFDYWGQGTTVTVSS

SEQ ID NO: 26 - protein, 451 aa, Artificial Sequence
  RB00005 HC
EVQLVQSGAEVKKPGASVKVSCKASGYTFTKYIIHWVRQAPGQGLEWMGWFYPGSGNIRYNEKIKGRVTMTRDTSTSTVYMELSSLRSEDTAVYYCARHGELGGGYFFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - protein, 10 aa, Artificial Sequence
  RB00005 LCDR1
  misc_feature (5)..(5): Xaa = Gly, Lys or Ser
  misc_feature (6)..(6): Xaa = Ala, Thr or Val
  misc_feature (7)..(7): Xaa = Gly or Ser
RASSXXXNIH

SEQ ID NO: 28 - protein, 10 aa, Artificial Sequence
  RB00005a LCDR1
RASSSVSNIH

SEQ ID NO: 29 - protein, 10 aa, Artificial Sequence
  RB00005b LCDR1
RASSKTGNIH

SEQ ID NO: 30 - protein, 10 aa, Artificial Sequence
  RB00005c LCDR1
RASSGASNIH

SEQ ID NO: 31 - protein, 7 aa, Artificial Sequence
  RB00005 LCDR2
ATSNLAS

SEQ ID NO: 32 - protein, 9 aa, Artificial Sequence
  RB00005 LCDR3
QQWSSNPLT

SEQ ID NO: 33 - protein, 23 aa, Artificial Sequence
  RB00005 LFR1
DIQLTQSPSFLSASVGDRVTITC

SEQ ID NO: 34 - protein, 15 aa, Artificial Sequence
  RB00005 LFR2
WYQQKPGKAPKPWIY

SEQ ID NO: 35 - protein, 32 aa, Artificial Sequence
  RB00005 LFR3
GVPSRFSGSGSGTEFTLTISSLQPEDFATYYC

SEQ ID NO: 36 - protein, 11 aa, Artificial Sequence
  RB00005 LFR4
FGQGTKLEIKR

SEQ ID NO: 37 - protein, 107 aa, Artificial Sequence
  RB00005 VL
  misc_feature (28)..(28): Xaa = Gly, Lys or Ser
  misc_feature (29)..(29): Xaa = Ala, Thr or Val
  misc_feature (30)..(30): Xaa = Gly or Ser
DIQLTQSPSFLSASVGDRVTITCRASSXXXNIHWYQQKPGKAPKPWIYATSNLASGVPSRFSGSGSGTEFTLTISSLQPEDFATYYCQQWSSNPLTFGQGTKLEIKR

SEQ ID NO: 38 - protein, 107 aa, Artificial Sequence
  RB00005a VL
DIQLTQSPSFLSASVGDRVTITCRASSSVSNIHWYQQKPGKAPKPWIYATSNLASGVPSRFSGSGSGTEFTLTISSLQPEDFATYYCQQWSSNPLTFGQGTKLEIKR

SEQ ID NO: 39 - protein, 107 aa, Artificial Sequence
  RB00005b VL
DIQLTQSPSFLSASVGDRVTITCRASSKTGNIHWYQQKPGKAPKPWIYATSNLASGVPSRFSGSGSGTEFTLTISSLQPEDFATYYCQQWSSNPLTFGQGTKLEIKR

SEQ ID NO: 40 - protein, 107 aa, Artificial Sequence
  RB00005c VL
DIQLTQSPSFLSASVGDRVTITCRASSGASNIHWYQQKPGKAPKPWIYATSNLASGVPSRFSGSGSGTEFTLTISSLQPEDFATYYCQQWSSNPLTFGQGTKLEIKR

SEQ ID NO: 41 - protein, 213 aa, Artificial Sequence
  RB00005 LC
  misc_feature (28)..(28): Xaa = Gly, Lys or Ser
  misc_feature (29)..(29): Xaa = Ala, Thr or Val
  misc_feature (30)..(30): Xaa = Gly or Ser
DIQLTQSPSFLSASVGDRVTITCRASSXXXNIHWYQQKPGKAPKPWIYATSNLASGVPSRFSGSGSGTEFTLTISSLQPEDFATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 213 aa, Artificial Sequence
  RB00005a LC
DIQLTQSPSFLSASVGDRVTITCRASSSVSNIHWYQQKPGKAPKPWIYATSNLASGVPSRFSGSGSGTEFTLTISSLQPEDFATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 213 aa, Artificial Sequence
  RB00005b LC
DIQLTQSPSFLSASVGDRVTITCRASSKTGNIHWYQQKPGKAPKPWIYATSNLASGVPSRFSGSGSGTEFTLTISSLQPEDFATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 213 aa, Artificial Sequence
  RB00005c LC
DIQLTQSPSFLSASVGDRVTITCRASSGASNIHWYQQKPGKAPKPWIYATSNLASGVPSRFSGSGSGTEFTLTISSLQPEDFATYYCQQWSSNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC